(19) United States
(12) Patent Application Publication
(10) Pub. No.: US 2025/0243544 A1
(43) Pub. Date: Jul. 31, 2025

(54) BIOMARKERS THAT PREDICT CLINICAL RESPONSE OR REMISSION IN ASTHMA PATIENTS

(71) Applicant: SANOFI US SERVICES INC., Bridgewater, NJ (US)

(72) Inventors: Lu Zhang, Cambridge, MA (US); David Habiel, Cambridge, MA (US); Eric E. Schadt, New York, NY (US); Jun Zhu, New York, NY (US); Linda Rogers, New York, NY (US); Ke Hao, New York, NY (US); Eunjee Lee, New York, NY (US)

(21) Appl. No.: 18/989,300

(22) Filed: Dec. 20, 2024

Related U.S. Application Data
(60) Provisional application No. 63/664,813, filed on Jun. 27, 2024.
(60) Provisional application No. 63/613,428, filed on Dec. 21, 2023.

(51) Int. Cl. C12Q 1/6883; C12Q 1/6874

(52) U.S. Cl. CPC C12Q 1/6883; C12Q 1/6874; C12Q 2600/106; C12Q 2600/158

(57) ABSTRACT

Methods for predicting whether a subject with asthma will respond to asthma therapy and/or will achieve asthma remission using biomarkers are provided. Methods of treating asthma in a subject with an increased expression level of at least one biomarker or a biomarker set are provided. Methods for predicting whether a subject with asthma will respond to asthma therapy and/or will achieve asthma by measuring ciliated cell levels, e.g., epithelial ciliated cell levels and/or epithelial proximal ciliated cell levels, are provided.

### RELATED APPLICATIONS

This application claims priority to U.S. Provisional Patent Application Serial Nos. 63/613,428, filed Dec. 21, 2023, and 63/664,813, filed Jun. 27, 2024, the entire disclosures which are hereby incorporated by reference in their entireties for all purposes.

### FIELD OF THE INVENTION

This disclosure relates to the treatment and/or prevention of asthma. This disclosure more specifically relates to biomarkers that can be used to correlate the outcome of asthma treatment with a biologic compound in a subject in need thereof, as well as a way to identify subjects for asthma treatment with one or more biologic compounds.

### BACKGROUND

Asthma is a chronic inflammatory heterogeneous disease of the airways characterized by airway hyper responsiveness, acute and chronic bronchoconstriction, airway edema and mucus plugging. The inflammation component of asthma is thought to involve many cell types, including mast cells, eosinophils, T lymphocytes, neutrophils, epithelial cells, and their biological products. Patients with asthma most often present with symptoms of wheezing, shortness of breath, cough, and chest tightness. For most asthma patients, a regimen of controller therapy and bronchodilator therapy provides adequate long-term control. Inhaled corticosteroids (ICS) are considered the “gold standard” in controlling asthma symptoms, and inhaled beta2-agonists are the most effective bronchodilators currently available. Studies have shown that combination therapy of an ICS with an inhaled long-acting beta2-agonist (LABA) provides better asthma control than high doses of ICS alone. Consequently, combination therapy has been the recommended treatment for subjects who are not controlled on low doses of ICS alone.

Nonetheless, it is estimated that 5% to 10% of the population with asthma has symptomatic disease despite maximum recommended treatment with combinations of anti-inflammatory and bronchodilator drugs.

Asthma remains a difficult-to-manage disease that is typically treated using empirical approaches based on disease severity rather than an understanding of the underlying mechanisms of pathogenesis. Although several biologic compounds, e.g., antibodies, are currently available to treat asthma, they may not work for all asthma patients. Accordingly, a need exists for elucidating a mechanistic understanding of asthma in order to provide precision treatment of asthma with a biologic compound.

### BRIEF SUMMARY OF THE INVENTION

In certain aspects, a method of treating a chronic airway disease in a subject is provided, comprising selecting a subject having increased goblet cell expression relative to a control and administering to the subject a drug that promotes transdifferentiation of goblet cells into ciliated cells.

In certain exemplary embodiments, the chronic airway disease is selected from the group consisting of asthma, chronic rhinosinusitis, allergic rhinitis, allergic fungal rhinosinusitis, chronic sinusitis, allergic bronchopulmonary aspergillosis (ABPA), bronchiectasis, unified airway disease, eosinophilic granulomatosis with polyangiitis (EGPA), gastroesophageal reflux disease (GERD), cystic fibrosis (CF), chronic obstructive pulmonary disease (COPD), eosinophilic esophagitis (EoE), chronic rhinosinusitis with nasal polyps (CRSwNP), chronic rhinosinusitis without nasal polyps (CRSsNP), aspirin hypersensitivity, NSAID exacerbated respiratory disease (NSAID-ERD), perennial allergic rhinitis (PAR), food allergy, and chronic eosinophilic pneumonia (CEP). In certain exemplary embodiments, the chronic airway disease is asthma.

In certain exemplary embodiments, the drug is a biologic compound that is selected from the group consisting of dupilumab, benralizumab, mepolizumab, omalizumab, reslizumab and tezepelumab.

In certain exemplary embodiments, goblet cells are hyperplastic goblet cells. In certain exemplary embodiments, the ciliated cells are mucous ciliated cells.

In certain exemplary embodiments, airway damage is reversed in the subject after administering the drug.

In certain exemplary embodiments, the subject achieves a decreased Global Initiative for Asthma (GINA) score, an increased asthma control test (ACT) score, or a decrease in number of exacerbations within six months of administering the drug.

In certain exemplary embodiments, the subject achieves asthma remission.

In certain aspects, a method of determining whether a subject is a suitable candidate for drug treatment for a chronic airway disease is provided, comprising obtaining a pre-treatment sample from the subject, treating the subject with the drug, and obtaining a post-treatment sample from the subject, wherein the subject is determined to be a suitable candidate for treatment if mucous-ciliated cell frequency is increased in the post-treatment sample relative to the pre-treatment sample.

In certain exemplary embodiments, the pre-treatment and post-treatment samples are nasal brush samples from the subject.

In certain exemplary embodiments, the chronic airway disease is selected from the group consisting of asthma, chronic rhinosinusitis, allergic rhinitis, allergic fungal rhinosinusitis, chronic sinusitis, ABPA, bronchiectasis, unified airway disease, EGPA, GERD, CF, COPD, EoE, CRSwNP, CRSsNP, aspirin hypersensitivity, NSAID-ERD, PAR, food allergy, and CEP. In certain exemplary embodiments, the chronic airway disease is asthma.

In certain exemplary embodiments, the drug is a biologic compound that is selected from the group consisting of dupilumab, benralizumab, mepolizumab, omalizumab, reslizumab and tezepelumab.

In certain exemplary embodiments, the goblet cells are hyperplastic goblet cells. In certain exemplary embodiments, the ciliated cells are mucous ciliated cells.

In certain aspects, a method of monitoring efficacy of drug treatment in a subject having a chronic airway disease is provided, comprising obtaining a pre-treatment sample from the subject, treating the subject with the drug, and obtaining two or more post-treatment samples from the subject, wherein the drug is determined to be efficacious if mucous-ciliated cell frequency remains increased in the post-treatment samples relative to the pre-treatment sample.

In certain exemplary embodiments, the pre-treatment and post-treatment samples are nasal brush samples from the subject.

In certain exemplary embodiments, the chronic airway disease is selected from the group consisting of asthma, chronic rhinosinusitis, allergic rhinitis, allergic fungal rhinosinusitis, chronic sinusitis, ABPA, bronchiectasis, unified airway disease, EGPA, GERD, CF, COPD, EoE, CRSwNP, CRSsNP, aspirin hypersensitivity, NSAID-ERD, PAR, food allergy, and CEP. In certain exemplary embodiments, the chronic airway disease is asthma.

In certain exemplary embodiments, the drug is a biologic compound that is selected from the group consisting of dupilumab, benralizumab, mepolizumab, omalizumab, reslizumab and tezepelumab.

In certain exemplary embodiments, the goblet cells are hyperplastic goblet cells. In certain exemplary embodiments, the ciliated cells are mucous ciliated cells.

In certain aspects, a method of screening whether a drug is a suitable treatment for a chronic airway disease is provided, comprising contacting goblet cells with the drug, wherein the drug stimulates transdifferentiation of goblet cells into ciliated cells if the drug is suitable for treating the chronic airway disease.

In certain exemplary embodiments, the goblet cells are obtained from nasal brush samples from the subject. In certain exemplary embodiments, the goblet cells are grown in vitro.

In certain exemplary embodiments, the chronic airway disease is selected from the group consisting of asthma, chronic rhinosinusitis, allergic rhinitis, allergic fungal rhinosinusitis, chronic sinusitis, ABPA, bronchiectasis, unified airway disease, EGPA, GERD, CF, COPD, EoE, CRSwNP, CRSsNP, aspirin hypersensitivity, NSAID-ERD, PAR, food allergy, and CEP. In certain exemplary embodiments, the chronic airway disease is asthma.

In certain exemplary embodiments, the drug is a biologic compound that is selected from the group consisting of dupilumab, benralizumab, mepolizumab, omalizumab, reslizumab and tezepelumab.

In certain exemplary embodiments, the goblet cells are hyperplastic goblet cells. In certain exemplary embodiments, the ciliated cells are mucous ciliated cells.

In another aspect, a bioassay to screen whether a drug is a suitable treatment for a chronic airway disease in a subject is provided, comprising contacting goblet cells from the subject with the drug, wherein the drug stimulates transdifferentiation of goblet cells into ciliated cells if the drug is suitable for treating the chronic airway disease.

In certain exemplary embodiments, the drug is administered directly to the subject.

In certain exemplary embodiments, the goblet cells are obtained from nasal brush samples from the subject.

In certain exemplary embodiments, the goblet cells are obtained from nasal brush samples from the subject prior to administering the drug and grown in vitro. In certain exemplary embodiments, the goblet cells grown in vitro are contacted with the drug.

In certain exemplary embodiments, the chronic airway disease is selected from the group consisting of asthma, chronic rhinosinusitis, allergic rhinitis, allergic fungal rhinosinusitis, chronic sinusitis, ABPA, bronchiectasis, unified airway disease, EGPA, GERD, CF, COPD, EoE, CRSwNP, CRSsNP, aspirin hypersensitivity, NSAID-ERD, PAR, food allergy, and CEP. In certain exemplary embodiments, the chronic airway disease is asthma.

In certain exemplary embodiments, the drug is a biologic compound that is selected from the group consisting of dupilumab, benralizumab, mepolizumab, omalizumab, reslizumab and tezepelumab.

In certain exemplary embodiments, the goblet cells are hyperplastic goblet cells. In certain exemplary embodiments, the ciliated cells are mucous ciliated cells.

In certain aspects, a method for predicting whether a subject with asthma will respond to asthma therapy is provided, comprising measuring an expression level of at least one gene, at least two genes, at least three genes, at least four genes, at least five genes, at least six genes, at least seven genes, at least eight genes, at least nine genes, at least ten genes, at least eleven genes, at least twelve genes, at least thirteen genes, at least fourteen genes, at least fifteen genes, at least sixteen genes, at least seventeen genes, at least eighteen genes, or at least nineteen genes selected from the group consisting of C20orf85, FAM92B, CAPS, MORN5, CAPSL, ZMYND10, SNTN, TCTEX1D2, PIFO, RSPH1, DYNLRB2, C2orf40, SPA17, CCDC170, C11orf88, CETN2, ROPN1L, CCDC78 and C9orf24 in a biological sample from the subject, wherein an increase in expression level of at least one gene, at least two genes, at least three genes, at least four genes, at least five genes, at least six genes, at least seven genes, at least eight genes, at least nine genes, at least ten genes, at least eleven genes, at least twelve genes, at least thirteen genes, at least fourteen genes, at least fifteen genes, at least sixteen genes, at least seventeen genes, at least eighteen genes, or at least nineteen genes selected from the group consisting of C20orf85, FAM92B, CAPS, MORN5, CAPSL, ZMYND10, SNTN, TCTEX1D2, PIFO, RSPH1, DYNLRB2, C2orf40, SPA17, CCDC170, C11orf88, CETN2, ROPN1L, CCDC78 and C9orf24 relative to a control is an indication that the subject will respond to the asthma therapy.

In certain exemplary embodiments, the asthma therapy comprises administering a biologic compound to the subject. In certain exemplary embodiments, the biologic compound is selected from the group consisting of dupilumab, benralizumab, mepolizumab, omalizumab, reslizumab and tezepelumab.

In certain exemplary embodiments, the asthma therapy increases Asthma Control Test (ACT) score, reduces Global Initiative for Asthma (GINA) score, and/or decreases risk of exacerbations in the subject.

In certain exemplary embodiments, the biological sample is a nasal brush sample.

In certain aspects, a method for predicting whether a subject with asthma will achieve asthma remission in response to asthma therapy is provided, comprising measuring an expression level of at least one gene, at least two genes, at least three genes, at least four genes, at least five genes, at least six genes, at least seven genes, at least eight genes, at least nine genes, at least ten genes, at least eleven genes, at least twelve genes, at least thirteen genes, at least fourteen genes, at least fifteen genes, at least sixteen genes, at least seventeen genes, at least eighteen genes, or at least nineteen genes selected from the group consisting of C20orf85, FAM92B, CAPS, MORN5, CAPSL, ZMYND10, SNTN, TCTEX1D2, PIFO, RSPH1, DYNLRB2, C2orf40, SPA17, CCDC170, C11orf88, CETN2, ROPN1L, CCDC78 and C9orf24 in a biological sample from the subject, wherein an increase in expression level of at least one gene, at least two genes, at least three genes, at least four genes, at least five genes, at least six genes, at least seven genes, at least eight genes, at least nine genes, at least ten genes, at least eleven genes, at least twelve genes, at least thirteen genes, at least fourteen genes, at least fifteen genes, at least sixteen genes, at least seventeen genes, at least eighteen genes, or at least nineteen genes selected from the group consisting of C20orf85, FAM92B, CAPS, MORN5, CAPSL, ZMYND10, SNTN, TCTEX1D2, PIFO, RSPH1, DYNLRB2, C2orf40, SPA17, CCDC170, C11orf88, CETN2, ROPN1L, CCDC78 and C9orf24 relative to a control is an indication that the subject will achieve asthma remission from the asthma therapy.

In certain exemplary embodiments, the asthma therapy comprises administering a biologic compound to the subject. In certain exemplary embodiments, the biologic compound is selected from the group consisting of dupilumab, benralizumab, mepolizumab, omalizumab, reslizumab and tezepelumab.

In certain exemplary embodiments, the asthma therapy increases ACT score, reduces GINA score, and/or decreases risk of exacerbations in the subject.

In certain exemplary embodiments, the biological sample is a nasal brush sample.

In certain aspects, a method of treating asthma in a subject with an increased expression level of at least one gene, at least two genes, at least three genes, at least four genes, at least five genes, at least six genes, at least seven genes, at least eight genes, at least nine genes, at least ten genes, at least eleven genes, at least twelve genes, at least thirteen genes, at least fourteen genes, at least fifteen genes, at least sixteen genes, at least seventeen genes, at least eighteen genes, or at least nineteen genes selected from the group consisting of C11orf88, C20orf85, C2orf40, C9orf24, CAPS, CAPSL, CCDC170, CCDC78, CETN2, DYNLRB2, FAM92B, MORN5, PIFO, ROPN1L, RSPH1, SNTN, SPA17, TCTEX1D2 and ZMYND10 in a biological sample obtained from the subject relative to a control is provided, comprising selecting the subject with the increased expression level and administering to the subject asthma therapy.

In certain exemplary embodiments, the asthma therapy comprises administering a biologic compound to the subject. In certain exemplary embodiments, the biologic compound is selected from the group consisting of dupilumab, benralizumab, mepolizumab, omalizumab, reslizumab and tezepelumab.

In certain exemplary embodiments, the asthma therapy increases ACT score, reduces GINA score, and/or decreases risk of exacerbations in the subject.

In certain exemplary embodiments, the biological sample is a nasal brush sample.

In certain aspects, a method for predicting whether a subject with asthma will achieve asthma remission in response to asthma therapy is provided, comprising measuring a ciliated cell gene signature in a biological sample from the subject, wherein an increase in the ciliated cell gene signature expression levels observed relative to a control is an indication that the subject will achieve asthma remission from the asthma therapy.

In certain exemplary embodiments, the ciliated cell gene signature is an epithelial ciliated signature. In certain exemplary embodiments, the epithelial ciliated signature comprises at least one gene, at least one gene, at least two genes, at least three genes, at least four genes, at least five genes, at least six genes, at least seven genes, at least eight genes, at least nine genes, at least ten genes, at least eleven genes, at least twelve genes, at least thirteen genes, or at least fourteen genes selected from the group consisting of FAM92B, CAPSL, C20orf85, C11orf88, SNTN, ROPN1L, ZMYND10, C9orf24, MORN5, CCDC78, CCDC170, RSPH9, C9orf116 and RSPH1.

In certain exemplary embodiments, the ciliated cell gene signature is an epithelial proximal ciliated signature. In certain exemplary embodiments, the epithelial proximal ciliated signature comprises, at least one gene, at least two genes, at least three genes, at least four genes, at least five genes, at least six genes, at least seven genes, at least eight genes, at least nine genes, at least ten genes, at least eleven genes, at least twelve genes, at least thirteen genes, at least fourteen genes, at least fifteen genes, at least sixteen genes, at least seventeen genes, at least eighteen genes, at least nineteen genes, at least twenty genes, at least twenty one genes, or at least twenty two genes selected from the group consisting of CAPSL, C20orf85, SNTN, CAPS, ROPN1L, PIFO, ZMYND10, C9orf24, MORN5, C2orf40, WDR54, CRIP1, DYNLRB2, SPA17, CES1, C9orf116, RSPH1, TCTEX1D2, TSPAN1, CETN2, TUBB4B and DYNLT1.

In certain exemplary embodiments, ciliated cell frequency is increased in the subject relative to the control.

In certain exemplary embodiments, the asthma therapy comprises administering a biologic compound to the subject. In certain exemplary embodiments, the biologic compound is selected from the group consisting of dupilumab, benralizumab, mepolizumab, omalizumab, reslizumab and tezepelumab.

In certain exemplary embodiments, the asthma therapy increases ACT score, reduces GINA score, and/or decreases risk of exacerbations in the subject.

In certain exemplary embodiments, the biological sample is a nasal brush sample.

In certain exemplary embodiments, the ciliated cell gene signature comprises, at least one gene, at least two genes, at least three genes, at least four genes, at least five genes, or at least six genes selected from the group consisting of FAM92B, CAPSL, C20orf85, C11orf88, CAPS and RSPH9.

In certain aspects, a method of treating asthma in a subject with an increased expression level of a ciliated cell gene signature in a biological sample obtained from the subject relative to a control is provided, comprising selecting the subject with an increased ciliated cell gene signature and administering to the subject asthma therapy.

In certain exemplary embodiments, the ciliated cell gene signature is an epithelial ciliated signature. In certain exemplary embodiments, the epithelial ciliated signature comprises, at least one gene, at least two genes, at least three genes, at least four genes, at least five genes, at least six genes, at least seven genes, at least eight genes, at least nine genes, at least ten genes, at least eleven genes, at least twelve genes, at least thirteen genes, or at least fourteen genes selected from the group consisting of FAM92B, CAPSL, C20orf85, C11orf88, SNTN, ROPN1L, ZMYND10, C9orf24, MORN5, CCDC78, CCDC170, RSPH9, C9orf116 and RSPH1.

In certain exemplary embodiments, the ciliated cell gene signature is an epithelial proximal ciliated signature.

In certain exemplary embodiments, the epithelial proximal ciliated signature comprises, at least one gene, at least two genes, at least three genes, at least four genes, at least five genes, at least six genes, at least seven genes, at least eight genes, at least nine genes, at least ten genes, at least eleven genes, at least twelve genes, at least thirteen genes, at least fourteen genes, at least fifteen genes, at least sixteen genes, at least seventeen genes, at least eighteen genes, at least nineteen genes, at least twenty genes, at least twenty-one genes, or at least twenty-two genes selected from the group consisting of CAPSL, C20orf85, SNTN, CAPS, ROPN1L, PIFO, ZMYND10, C9orf24, MORN5, C2orf40, WDR54, CRIP1, DYNLRB2, SPA17, CES1, C9orf116, RSPH1, TCTEX1D2, TSPAN1, CETN2, TUBB4B and DYNLT1.

In certain exemplary embodiments, ciliated cell frequency is increased in the subject relative to the control.

In certain exemplary embodiments, the asthma therapy comprises administering a biologic compound to the subject. In certain exemplary embodiments, the biologic compound is selected from the group consisting of dupilumab, benralizumab, mepolizumab, omalizumab, reslizumab and tezepelumab.

In certain exemplary embodiments, the asthma therapy increases ACT score, reduces GINA score, and/or decreases risk of exacerbations in the subject.

In certain exemplary embodiments, the biological sample is a nasal brush sample.

In certain exemplary embodiments, the ciliated cell gene signature comprises, at least one gene, at least two genes, at least three genes, at least four genes, at least five genes, or at least six genes selected from the group consisting of FAM92B, CAPSL, C20orf85, C11orf88, CAPS and RSPH9.

In certain aspects, a method for treating asthma in a patient in need thereof is provided, comprising administering a biologic compound to the patient, wherein the patient has been identified as having a serum, plasma, blood or nasal brush sample level of at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least eleven biomarkers, at least twelve biomarkers, at least thirteen biomarkers, at least fourteen biomarkers, at least fifteen biomarkers, at least sixteen biomarkers, at least seventeen biomarkers, at least eighteen biomarkers, or at least nineteen biomarkers selected from the group consisting of C20orf85, FAM92B, CAPS, MORN5, CAPSL, ZMYND10, SNTN, TCTEX1D2, PIFO, RSPH1, DYNLRB2, C2orf40, SPA17, CCDC170, C11orf88, CETN2, ROPN1L, CCDC78 and C9orf24 that is increased relative to a control.

In certain exemplary embodiments, the biologic compound is selected from the group consisting of dupilumab, benralizumab, mepolizumab, omalizumab, reslizumab and tezepelumab.

In certain aspects, a method for treating asthma in a patient in need thereof is provided, comprising administering a biologic compound to the patient, wherein the patient has been identified as having a serum, plasma, blood or nasal brush sample level of at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least eleven biomarkers, at least twelve biomarkers, at least thirteen biomarkers, at least fourteen biomarkers, at least fifteen biomarkers, at least sixteen biomarkers, at least seventeen biomarkers, at least eighteen biomarkers, or at least nineteen biomarkers selected from the group consisting of C11orf88, C20orf85, C2orf40, C9orf24, CAPS, CAPSL, CCDC170, CCDC78, CETN2, DYNLRB2, FAM92B, MORN5, PIFO, ROPN1L, RSPH1, SNTN, SPA17, TCTEX1D2 and ZMYND10 that is increased relative to a control.

In certain exemplary embodiments, the biologic compound is selected from the group consisting of dupilumab, benralizumab, mepolizumab, omalizumab, reslizumab and tezepelumab.

In certain aspects, a method for treating asthma in a patient in need thereof is provided, comprising administering a biologic compound to the patient, wherein the patient has been identified as having a serum, plasma, blood or nasal brush sample level of at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least eleven biomarkers, at least twelve biomarkers, at least thirteen biomarkers, at least fourteen biomarkers selected from the group consisting of FAM92B, CAPSL, C20orf85, C11orf88, SNTN, ROPN1L, ZMYND10, C9orf24, MORN5, CCDC78, CCDC170, RSPH9, C9orf116 and RSPH1 that is increased relative to a control.

In certain exemplary embodiments, the biologic compound is selected from the group consisting of dupilumab, benralizumab, mepolizumab, omalizumab, reslizumab and tezepelumab.

In certain aspects, a method for treating asthma in a patient in need thereof is provided, comprising administering a biologic compound to the patient, wherein the patient has been identified as having a serum, plasma, blood or nasal brush sample level of at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least eleven biomarkers, at least twelve biomarkers, at least thirteen biomarkers, at least fourteen biomarkers, at least fifteen biomarkers, at least sixteen biomarkers, at least seventeen biomarkers, at least eighteen biomarkers, at least nineteen biomarkers, at least twenty biomarkers, at least twenty-one biomarkers, or at least twenty-two biomarkers selected from the group consisting of CAPSL, C20orf85, SNTN, CAPS, ROPN1L, PIFO, ZMYND10, C9orf24, MORN5, C2orf40, WDR54, CRIP1, DYNLRB2, SPA17, CES1, C9orf116, RSPH1, TCTEX1D2, TSPAN1, CETN2, TUBB4B and DYNLT1 that is increased relative to a control.

In certain exemplary embodiments, the biologic compound is selected from the group consisting of dupilumab, benralizumab, mepolizumab, omalizumab, reslizumab and tezepelumab.

A method for treating asthma in a patient in need thereof, comprising administering a biologic compound to the patient, wherein the patient has been identified as having a serum, plasma, blood or nasal brush sample level of at least one biomarker, at least two biomarkers, at least three biomarkers, at least four biomarkers, at least five biomarkers, or at least six biomarkers selected from the group consisting of FAM92B, CAPSL, C20orf85, C11orf88, CAPS and RSPH9 that is increased relative to a control.

In certain exemplary embodiments, the biologic compound is selected from the group consisting of dupilumab, benralizumab, mepolizumab, omalizumab, reslizumab and tezepelumab.

In certain aspects, a method for treating asthma in a patient in need thereof is provided, comprising administering a biologic compound to the patient, wherein the patient has been identified as having an increased chance of benefiting from treatment with the biologic compound with an assay, wherein the assay comprises detecting at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least eleven biomarkers, at least twelve biomarkers, at least thirteen biomarkers, at least fourteen biomarkers, at least fifteen biomarkers, at least sixteen biomarkers, at least seventeen biomarkers, at least eighteen biomarkers, or at least nineteen biomarkers selected from the group consisting of C20orf85, FAM92B, CAPS, MORN5, CAPSL, ZMYND10, SNTN, TCTEX1D2, PIFO, RSPH1, DYNLRB2, C2orf40, SPA17, CCDC170, C11orf88, CETN2, ROPN1L, CCDC78 and C9orf24 that is increased relative to a control.

In certain exemplary embodiments, the biologic compound is selected from the group consisting of dupilumab, benralizumab, mepolizumab, omalizumab, reslizumab and tezepelumab.

In certain aspects, a method for treating asthma in a patient in need thereof is provided, comprising administering a biologic compound to the patient, wherein the patient has been identified as having an increased chance of benefiting from treatment with the biologic compound with an assay, wherein the assay comprises detecting at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least eleven biomarkers, at least twelve biomarkers, at least thirteen biomarkers, at least fourteen biomarkers, at least fifteen biomarkers, at least sixteen biomarkers, at least seventeen biomarkers, at least eighteen biomarkers, or at least nineteen biomarkers selected from the group consisting of C11orf88, C20orf85, C2orf40, C9orf24, CAPS, CAPSL, CCDC170, CCDC78, CETN2, DYNLRB2, FAM92B, MORN5, PIFO, ROPN1L, RSPH1, SNTN, SPA17, TCTEX1D2 and ZMYND10 that is increased relative to a control.

In certain exemplary embodiments, the biologic compound is selected from the group consisting of dupilumab, benralizumab, mepolizumab, omalizumab, reslizumab and tezepelumab.

In certain aspects, a method for treating asthma in a patient in need thereof is provided, comprising administering a biologic compound to the patient, wherein the patient has been identified as having an increased chance of benefiting from treatment with the biologic compound with an assay, wherein the assay comprises detecting at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least eleven biomarkers, at least twelve biomarkers, at least thirteen biomarkers, at least fourteen biomarkers selected from the group consisting of FAM92B, CAPSL, C20orf85, C11orf88, SNTN, ROPN1L, ZMYND10, C9orf24, MORN5, CCDC78, CCDC170, RSPH9, C9orf116 and RSPH1 that is increased relative to a control.

In certain exemplary embodiments, the biologic compound is selected from the group consisting of dupilumab, benralizumab, mepolizumab, omalizumab, reslizumab and tezepelumab.

In certain aspects, a method for treating asthma in a patient in need thereof is provided, comprising administering a biologic compound to the patient, wherein the patient has been identified as having an increased chance of benefiting from treatment with the biologic compound with an assay, wherein the assay comprises detecting at least one biomarker at least two biomarkers, at least three biomarkers, at least four biomarkers, at least five biomarkers, at least six biomarkers, at least seven biomarkers, at least eight biomarkers, at least nine biomarkers, at least ten biomarkers, at least eleven biomarkers, at least twelve biomarkers, at least thirteen biomarkers, at least fourteen biomarkers, at least fifteen biomarkers, at least sixteen biomarkers, at least seventeen biomarkers, at least eighteen biomarkers, at least nineteen biomarkers, at least twenty biomarkers, at least twenty-one biomarkers, or at least twenty-two biomarkers selected from the group consisting of CAPSL, C20orf85, SNTN, CAPS, ROPN1L, PIFO, ZMYND10, C9orf24, MORN5, C2orf40, WDR54, CRIP1, DYNLRB2, SPA17, CES1, C9orf116, RSPH1, TCTEX1D2, TSPAN1, CETN2, TUBB4B and DYNLT1 that is increased relative to a control.

In certain exemplary embodiments, the biologic compound is selected from the group consisting of dupilumab, benralizumab, mepolizumab, omalizumab, reslizumab and tezepelumab.

In certain aspects, a method for treating asthma in a patient in need thereof is provided, comprising administering a biologic compound to the patient, wherein the patient has been identified as having an increased chance of benefiting from treatment with the biologic compound with an assay, wherein the assay comprises detecting at least one biomarker, at least two biomarkers, at least three biomarkers, at least four biomarkers, at least five biomarkers, or at least six biomarkers selected from the group consisting of FAM92B, CAPSL, C20orf85, C11orf88, CAPS and RSPH9 that is increased relative to a control.

In certain exemplary embodiments, the biologic compound is selected from the group consisting of dupilumab, benralizumab, mepolizumab, omalizumab, reslizumab and tezepelumab.

In certain aspects, a method for treating asthma in a patient in need thereof is provided, wherein one or more biomarkers, such as at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least eleven biomarkers, at least twelve biomarkers, at least thirteen biomarkers, at least fourteen biomarkers, at least fifteen biomarkers, at least sixteen biomarkers, at least seventeen biomarkers, at least eighteen biomarkers, or at least nineteen biomarkers are selected from the group consisting of C20orf85, FAM92B, CAPS, MORN5, CAPSL, ZMYND10, SNTN, TCTEX1D2, PIFO, RSPH1, DYNLRB2, C2orf40, SPA17, CCDC170, C11orf88, CETN2, ROPN1L, CCDC78 and C9orf24 are present in the patient's nasal brush sample at an increased level relative to a control.

In certain aspects, a method for treating asthma in a patient in need thereof is provided, wherein one or more biomarkers, such as at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least eleven biomarkers, at least twelve biomarkers, at least thirteen biomarkers, at least fourteen biomarkers, at least fifteen biomarkers, at least sixteen biomarkers, at least seventeen biomarkers, at least eighteen biomarkers, or at least nineteen biomarkers selected from the group consisting of C11orf88, C20orf85, C2orf40, C9orf24, CAPS, CAPSL, CCDC170, CCDC78, CETN2, DYNLRB2, FAM92B, MORN5, PIFO, ROPN1L, RSPH1, SNTN, SPA17, TCTEX1D2 and ZMYND10 are present in the patient's nasal brush sample at an increased level relative to a control.

In certain aspects, a method for treating asthma in a patient in need thereof is provided, wherein one or more biomarkers, such as at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least eleven biomarkers, at least twelve biomarkers, at least thirteen biomarkers, at least fourteen biomarkers selected from the group consisting of FAM92B, CAPSL, C20orf85, C11orf88, SNTN, ROPN1L, ZMYND10, C9orf24, MORN5, CCDC78, CCDC170, RSPH9, C9orf116 and RSPH1 are present in the patient's nasal brush sample at an increased level relative to a control.

In certain aspects, a method for treating asthma in a patient in need thereof is provided, wherein one or more biomarkers, such as at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least eleven biomarkers, at least twelve biomarkers, at least thirteen biomarkers, at least fourteen biomarkers, at least fifteen biomarkers, at least sixteen biomarkers, at least seventeen biomarkers, at least eighteen biomarkers, at least nineteen biomarkers, at least twenty biomarkers, at least twenty-one biomarkers, or at least twenty-two biomarkers selected from the group consisting of CAPSL, C20orf85, SNTN, CAPS, ROPN1L, PIFO, ZMYND10, C9orf24, MORN5, C2orf40, WDR54, CRIP1, DYNLRB2, SPA17, CES1, C9orf116, RSPH1, TCTEX1D2, TSPAN1, CETN2, TUBB4B and DYNLT1 are present in the patient's nasal brush sample at an increased level relative to a control.

In certain aspects, a method for treating asthma in a patient in need thereof is provided, wherein at least one biomarker, at least two biomarkers, at least three biomarkers, at least four biomarkers, at least five biomarkers, or at least six biomarkers are selected from the group consisting of FAM92B, CAPSL, C20orf85, C11orf88, CAPS and RSPH9 are present in the patient's nasal brush sample at an increased level relative to a control.

In certain aspects, a method of treating asthma is provided, comprising selecting a patient who has a serum, plasma, blood or nasal brush sample level of at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least eleven biomarkers, at least twelve biomarkers, at least thirteen biomarkers, at least fourteen biomarkers, at least fifteen biomarkers, at least sixteen biomarkers, at least seventeen biomarkers, at least eighteen biomarkers, or at least nineteen biomarkers selected from the group consisting of C20orf85, FAM92B, CAPS, MORN5, CAPSL, ZMYND10, SNTN, TCTEX1D2, PIFO, RSPH1, DYNLRB2, C2orf40, SPA17, CCDC170, C11orf88, CETN2, ROPN1L, CCDC78 and C9orf24 that is increased relative to a control, and then administering a biologic compound to the patient.

In certain exemplary embodiments, the biologic compound is selected from the group consisting of dupilumab, benralizumab, mepolizumab, omalizumab, reslizumab and tezepelumab.

In certain aspects, a method of treating asthma is provided, comprising selecting a patient who has a serum, plasma, blood or nasal brush sample level of at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least eleven biomarkers, at least twelve biomarkers, at least thirteen biomarkers, at least fourteen biomarkers, at least fifteen biomarkers, at least sixteen biomarkers, at least seventeen biomarkers, at least eighteen biomarkers, or at least nineteen biomarkers selected from the group consisting of C11orf88, C20orf85, C2orf40, C9orf24, CAPS, CAPSL, CCDC170, CCDC78, CETN2, DYNLRB2, FAM92B, MORN5, PIFO, ROPN1L, RSPH1, SNTN, SPA17, TCTEX1D2 and ZMYND10 that is increased relative to a control, and then administering a biologic compound to the patient.

In certain exemplary embodiments, the biologic compound is selected from the group consisting of dupilumab, benralizumab, mepolizumab, omalizumab, reslizumab and tezepelumab.

In certain aspects, a method of treating asthma is provided, comprising selecting a patient who has a serum, plasma, blood or nasal brush sample level of at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least eleven biomarkers, at least twelve biomarkers, at least thirteen biomarkers, at least fourteen biomarkers selected from the group consisting of FAM92B, CAPSL, C20orf85, C11orf88, SNTN, ROPN1L, ZMYND10, C9orf24, MORN5, CCDC78, CCDC170, RSPH9, C9orf116 and RSPH1 that is increased relative to a control, and then administering a biologic compound to the patient.

In certain exemplary embodiments, the biologic compound is selected from the group consisting of dupilumab, benralizumab, mepolizumab, omalizumab, reslizumab and tezepelumab.

In certain aspects, a method of treating asthma is provided, comprising selecting a patient who has a serum, plasma, blood or nasal brush sample level of at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least eleven biomarkers, at least twelve biomarkers, at least thirteen biomarkers, at least fourteen biomarkers, at least fifteen biomarkers, at least sixteen biomarkers, at least seventeen biomarkers, at least eighteen biomarkers, at least nineteen biomarkers, at least twenty biomarkers, at least twenty-one biomarkers, or at least twenty-two biomarkers selected from the group consisting of CAPSL, C20orf85, SNTN, CAPS, ROPN1L, PIFO, ZMYND10, C9orf24, MORN5, C2orf40, WDR54, CRIP1, DYNLRB2, SPA17, CES1, C9orf116, RSPH1, TCTEX1D2, TSPAN1, CETN2, TUBB4B and DYNLT1 that is increased relative to a control, and then administering a biologic compound to the patient.

In certain exemplary embodiments, the biologic compound is selected from the group consisting of dupilumab, benralizumab, mepolizumab, omalizumab, reslizumab and tezepelumab.

In certain aspects, a method of treating asthma is provided, comprising selecting a patient who has a serum, plasma, blood or nasal brush sample level of at least one biomarker, at least two biomarkers, at least three biomarkers, at least four biomarkers, at least five biomarkers, or at least six biomarkers selected from the group consisting of FAM92B, CAPSL, C20orf85, C11orf88, CAPS and RSPH9 that is increased relative to a control, and then administering a biologic compound to the patient.

In certain exemplary embodiments, the biologic compound is selected from the group consisting of dupilumab, benralizumab, mepolizumab, omalizumab, reslizumab and tezepelumab.

Other embodiments will become apparent from a review of the ensuing detailed description, drawings, tables and accompanying claims.

### BRIEF DESCRIPTION OF THE FIGURES

The foregoing and other features and advantages of the present invention will be more fully understood from the following detailed description of illustrative embodiments taken in conjunction with the accompanying drawings. The file of this patent contains at least one drawing/photograph executed in color. Copies of this patent with color drawing(s)/photograph(s) will be provided by the Office upon request and payment of the necessary fee.

FIG. 1A-FIG. 1D depicts the REGAIN study design. (A) depicts the number of asthma and healthy participants included in this interim analysis. (B) depicts data types profiled for each study participant. Solid lines indicate data presented here, dashed lines indicate data to be analyzed. The bar plots depict the distribution of DNA samples and nasal brushing RNA-Seq samples collected across study visits. (C) depicts three main objectives of this disclosure. (D) depicts an overview of network-centric data analysis.

FIG. 2A-FIG. 2C depict the clinical and molecular heterogeneity of asthma by therapy type and over time, from interim analysis of the REGAIN cohort. (A) depicts Sankey diagrams showing 6-month outcomes for the three biologic and 2 STEP therapy groups defined at baseline as shown in Table 1. (B) shows the definition of on-therapy clinical remission, as well as 6- and 18-month remission outcomes for the ‘T2 de novo’ patients. (C) depicts GSEA of curated asthma-related signatures identified candidate mechanisms underlying the heterogeneity in clinically defined asthma groups and clinical remission in ‘T2 de novo’ group. The top 6 rows indicate comparisons of the five pre-specified groups of study participants with asthma as defined in Table 1 to all healthy control participants at baseline (BL). ‘Case’ compares all the five groups of study participants with asthma to all controls. The sample sizes for these groups are n=259, 102, 171, 132, 33, and 54 for controls, ‘T2 low STEP’, ‘T2 STEP’, ‘T2 bio’, ‘T2 failed’, and ‘T2 de novo’, respectively. The 7th row describes comparisons between baseline remission/non-remission (R/NR) status within the ‘T2 de novo’ group (n=12 remission and n=28 non-remission). The functional categories of the gene sets are labeled on the top of the heatmap. The color scale encodes the normalized enrichment (NES) scores, while asterisks indicate significance with FDR <0.05. 34 selected significant signatures are shown. Note that the ‘Asthma Up’ and ‘Asthma Down’ gene sets are significantly up- and down-regulated genes for asthma, derived from a meta-analysis comparing bulk RNA samples in bronchial and nasal brushings between 355 asthma patients and 193 healthy individuals.

FIG. 3A-FIG. 3E depict an integrative network-based approach of the disclosure. (A) depicts an overview of the integrative network-based approach. A coexpression network was built and annotated with cell type marker genes. To prioritize the most impacted modules related to asthma and response to treatment, differential expression (DE) and differential connectivity (DC) analysis were performed. In order to assess whether connectivity between the most impacted module and other modules are associated with clinical remission, an integrative hybrid network comprised of coexpression networks and Bayesian networks with statistically inferred causal relationships was constructed. The integrative hybrid networks were used to assess inter-module connectivity. (B) depicts differential connectivity (DC) within module scores to compare remission and non-remission networks. Modules are sorted by their DC scores. Asterisk indicates significance with FDR <0.01 and two asterisks indicate FDR <0.001. The ‘black’ and ‘purple’ modules showed the strongest loss of connectivity in remission compared to non-remission. The color scale encodes the TOM values. (C) shows the ranking of modules by integrating DE and DC scores. The first three columns indicate expression comparisons between the baseline and post-biologic treatments within the R group of ‘T2 de novo,’ between baseline remission/non-remission (N/NR) status within the ‘T2 de novo’ group, and between asthma and control at baseline, respectively. The color scale for the first three columns encodes the absolute value of standardized normalized enrichment scores (NES). The fourth column indicates connectivity comparison between remission and non-remission networks. The color scale for the fourth column encodes the absolute value of standardized DC scores. The final column denotes the average of these four columns. Modules are ordered by the final column. Triangle indicates the module that met stringent enrichment cutoffs (FDR <0.001) across all DE and DC criteria. (D) shows differential connectivity between modules comparing remission and non-remission networks. The color scale encodes −log(P) from a two-sample Z test of proportion with directionality of loss/gain of connectivity. Blue and red indicate loss and gain of connectivity between modules in remission compared to non-remission networks, respectively. (E) depicts dynamic composition changes of mucous ciliated cells responding to treatments. Frequency of 18 cell types including mucous ciliated cells were inferred by in silico deconvolution of nasal brushing tissue. Mucous ciliated cell frequencies in heathy controls at baseline and over time in the ‘T2 de novo’ group participants are shown in remission and non-remission at 6 months.

FIG. 4 is a summary of a network-centric analysis-identified novel primary regulatory mechanism for both asthma and responses to biologics.

FIG. 5A-FIG. 5I depict an in vitro organoid model based on air-liquid interface cultured pseudostratified airway (ALI) epithelial cells according to certain aspects of the disclosure. Cultured cells were treated with: vehicle; IL-13 for 10 days; IL-13 for 7 days followed by 3 days of recovery (where the cytokine was washed off); or IL-13 for 4 days followed by 6 days of recovery. Here, IL-13 treatment mimics a type 2 inflammatory milieu, while removal of IL-13 mimics anti-IL-13 biologics treatment. (A) Single-cell suspensions (2 replicates per condition) were generated and analyzed by scRNA-Seq. (B) Uniform manifold approximation and projection (UMAP) visualization of single-cell profiles colored by treatment conditions. (C) UMAP visualization colored by assigned cell types. (D) Distribution of cell type frequencies across the four treatment conditions. (E)-(H) RNA velocity analysis inferred dynamic relationships among cell types in each treatment condition. (I) Summary of our findings related to therapeutic response to biologics treatment in type 2 asthma.

FIG. 6A-FIG. 6B depict the enrichment of co-expression modules based on curated asthma-related signatures (A) and cell type markers (B). The color scale indicates odds ratio. Asterisk indicates significance with FDR <0.01, and two asterisks indicate significance with FDR <0.001.

FIG. 7 depicts GSEA results for co-expression modules to link between each module and asthma disease signatures. The rows indicate comparisons of the five pre-specified groups of study participants with asthma as defined in Table 1 to healthy control participants at baseline (BL) as well as a comparison of ‘Case’ combing all the five asthma groups to healthy controls at baseline. The sample sizes for these groups are n=259, 102, 171, 132, 33, and 54 for controls, ‘T2 low STEP’, ‘T2 STEP’, ‘T2 bio’, ‘T2 failed’, and ‘T2 de novo’, respectively. The color scale encodes the normalized enrichment (NES) scores. Asterisk indicates significance with FDR <0.01, and two asterisks indicate significance with FDR <0.001.

FIG. 8 depicts GSEA results for co-expression modules to link between each module and treatment response. The first and second rows indicate comparisons of between the baseline and post-biologic (6 month) treatments within the remission group (n=12 baseline and n=8 post) and non-remission (n=28 baseline and n=19 post) group, respectively. The third row indicates comparisons between baseline remission/non-remission (R/NR) status within ‘T2 de novo’ group (n=12 remission and n=28 non-remission). The color scale encodes the normalized enrichment (NES) scores. Asterisk indicates significance with FDR <0.01, and two asterisks indicate significance with FDR <0.001.

FIG. 9 depicts differential connectivity between modules comparing remission and non-remission networks. The color scale encodes −log(P) from a two-sample Z test of proportion with directionality of loss/gain of connectivity. Blue and red indicate loss and gain of connectivity between modules in remission compared to non-remission networks, respectively.

FIG. 10A-FIG. 10B depict a PCA of module and cell type markers. (A) Correlation coefficients between module PCs for three modules and cell type marker PCs. The color scale indicates the absolute value of correlation coefficients. Asterisk indicates the absolute value of correlation is bigger than 0.5, and two asterisks indicate the cell type marker PC with most significant correlation. (B) Correlation coefficients among module PCs. Asterisk indicates significance with FDR <0.01 and two asterisks indicate significance with FDR <0.001.

FIG. 11 depicts the association between cell frequency and asthma groups. P values are from linear model. Cell frequencies of participants in each asthma group were compared to those of healthy control participants, and both groups and cell types were hierarchically clustered. The sample sizes for these groups are n=259, 102, 171, 132, 33, and 54 for controls, ‘T2 low STEP’, ‘T2 STEP’, ‘T2 bio’, ‘T2 failed’, and ‘T2 de novo’, respectively.

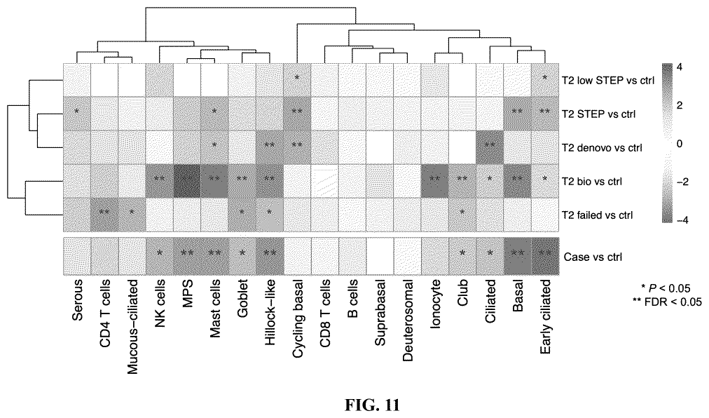

FIG. 12A-FIG. 12F depict distinct cell compositions of asthma groups. (A) Approach to in silico deconvolution of nasal brushing tissue. (B) Association of cell frequency with treatment and remission status in ‘T2 de novo’. The first row shows the association between baseline cell frequency and remission status. P values are from robust linear regression, with baseline ACT scores as covariate. The second to fourth rows show the effect of remission status, treatment, and their interaction on cell frequency within ‘T2 de novo’ groups. P values are from linear mixed-effects model across all time points. The sample sizes for these analyses are n=12 R and n=28 NR at baseline, n=9 R and n=18 NR at 3 months, n=8 R and n=19 NR at 6 months, and n=4 R and n=15 NR at 18 months. (C) Ciliated, (D) mucous-ciliated, (E) goblet, and (F) hillock-like cell frequencies in heathy controls at baseline and over time in the ‘T2 de novo’ group participants showing remission and non-remission at 6 months. In (E) and (F), P values were from Wilcoxon signed-rank test. The number of healthy controls is 258. The number of ‘T2 de novo’ samples is the same as in (B). In the boxplots, the center lines indicate the median and the lower and upper box limits represent the first and third quartiles of the data, respectively.

FIG. 13 illustrates the REGAIN multi-modal computational modeling framework to investigate asthma patients at phenotypic, molecular, cellular, and systems biology/network levels.

FIG. 14A-FIG. 14B depict ACT score trajectories in ‘T2 de novo’ study participants (n=44). (A) Displayed are the means and standard deviation of the ACT scores of each remission status category across study visits, where the remission status was defined based on 6-month visit. The remission groups (clinical remission and clinical remission on minimal medications) showed an increasing trend in average ACT score over time, increasing from 16.3 at baseline to 22.8 at 6 months and to 24.4 at 18 months (6 months versus Baseline, P=0.0026; 18 months versus Baseline, P=0.0054; Mann-Whitney U test). The non-remission groups (partial symptom or exacerbation control, uncontrolled symptoms, and uncontrolled symptoms+exacerbation) achieved modestly improved symptoms with average ACT scores of 12.9, 15.4, and 16.6 at baseline, 6 months, and 18 months, but remained uncontrolled (i.e., <20) during the study (6 months versus baseline P=0.014 and 18 months versus baseline P=0.011 from Mann-Whitney U test). The remission groups show a larger magnitude of improvement in ACT score than non-remission groups across all study visits (linear mixed model P=0.0012, 0.0044, and 0.0036 for the remission status, visit time, and interaction terms). The association between baseline ACT scores and remission status was also investigated. The difference was not significant (16.3 versus 12.9, Mann-Whitney U-test P=0.069). (B) The remission groups showed overall improved ACT scores, with significant higher values compared to non-remission groups at 18 months (P=0.00182, Mann-Whitney U-test). In the boxplot, the central line indicates the median; the lower and upper limits of the box represent the first and third quartiles of the data, respectively; the whiskers extend to 1.5 times the interquartile range (IQR).

FIG. 15 depicts a co-occurrence plot between member genes of the 116 curated asthma signatures. Color scale represents the Cramer's V score (between 0 and 1) for each pair of signatures, signed by the log odds ratio from right-sided Fisher's exact test. Signature labels were then ordered based on hierarchical clustering of the co-occurrence patterns (with R hclust function and average linkage). Low overlap in pairwise comparisons suggests that most of the 116 signatures reflect unique biological pathways and mechanisms.

FIG. 16 depicts a Pairwise Pearson's correlation plot between the 116 curated asthma signatures. Color scale represents the Pearson correlation coefficient (r) of each pair of signatures across all studied nasal brushing RNA-Seq samples, based on their GSVA scores. Signature labels were then ordered based on hierarchical clustering of the correlation patterns (with R hclust function and average linkage). Strong positive correlations were observed in some of the pairwise comparisons. Few signatures showed weak negative correlations with other signatures.

FIG. 17 depicts full GSEA results for curated asthma-related signatures. The first 6 columns indicate comparisons of the five pre-specified groups of study participants with asthma as defined in Table 1 to healthy control participants at baseline (BL) as well as a comparison of ‘Case’ combing all the five asthma groups to healthy controls at baseline. The 7th column describes comparisons between baseline remission/non-remission (R/NR) status within the de novo biologics group. Displayed are significant signatures in at least one comparison at Benjamini-Hochberg FDR <0.05. The functional categories of the gene sets are labeled on the left of the heatmaps. The color scale encodes the normalized enrichment (NES) scores, while asterisk indicates significance with FDR <0.05.

FIG. 18 depicts a heatmap visualization of the ciliated cell signature genes across ‘T2 de novo’ participants. Columns are samples (n=69) ordered by 6-month remission status (21 for remission and 48 for non-remission) and time points (40 from baseline and 29 from 6 months). Also shown on the top of the heatmap are the ACT and GINA scores of each patient at baseline, 6 months, 18 months, the five clinical remission status categories at 6 months and 18 months, and the biologic therapy that each patient was receiving throughout 6 months. Rows are 40 genes from the union of ‘epithelial ciliated’ (n=16) and ‘epithelial proximal ciliated’ (n=34) signatures. In the baseline predictive biomarker analysis comparing remission versus non-remission using GSEA, 14 genes from the ‘epithelial ciliated’ signature and 26 genes from the ‘epithelial proximal ciliated’ signature were detected as ‘leading-edge genes’, indicating they were driving these signatures' enrichment signals (epithelial ciliated signature: NES=2.36, P=3.3×10-13, FDR-adjusted P=3.2×10-11; and epithelial proximal ciliated: NES=2.23, P=8×10-10, FDR-adjusted P=2.7×10-8). A subset of them—CAPSL, CAPS, C11orf88, and RSPH9—had fold-change >2 and nominal P<0.05. Additionally, the predictive value of this 4-gene signature was confirmed using GSVA, followed by limma linear modeling. It was discovered that the sample-wise scores of this gene set at baseline were significantly higher in remission compared to non-remission (P=0.039). Heatmap color denotes the scaled expression (z-score) per gene across all nasal brushing samples profiled in REGAIN.

FIG. 19A-FIG. 19D depict cell type proportion inference using deconvolution. (A) UMAP based on scRNA-seq data of airway epithelium in people with asthma (GSE193816). (B) Boxplot of cell frequencies of 20 different cell types in all nasal brushing bulk RNA samples profiled in REGAIN, estimated using deconvolution with GSE193816 scRNA-Seq dataset as the reference. The center lines indicate the median and the lower and upper box limits represent the first and third quantiles, respectively, for each cell type. (C) Mucous-ciliated cell frequencies that corrected by batch, study site, age, and sex for each asthma group and healthy control. The boxplots reflect the medians (center line) and interquartile range (boxes) of all samples from all measured time points for each asthma group. (D) Ciliated cell frequencies that adjusted for the same covariates as in (C).

FIG. 20 depicts a GSEA analysis of cell type markers. Exclusive markers are those derived from Findmarkers and share no marker genes with other exclusive markers, but non-exclusive markers can share some marker genes between cell types. Each asthma group was compared to healthy control. ‘Case’ compares all study participants with asthma to all controls. The sample sizes for these groups are n=258, 102, 171, 132, 33, and 54 for controls, ‘T2 low STEP’, ‘T2 STEP’, ‘T2 bio’, ‘T2 failed’, and ‘T2 den ovo’, respectively. Remission versus non-remission patients among the ‘T2 de novo’ group were compared in terms of their baseline gene expression. Asterisk indicates significance with FDR <0.05 and two asterisks indicate FDR <0.01.

FIG. 21 depicts the enrichment of curated asthma-related signatures for marker genes of ciliated, basal, and goblet subcluster from scRNAseq ALI epithelial cell data. Goblet subcluster marker genes were identified as differentially expressed genes by comparison among goblet subclusters (i.e., goblet, goblet-B, goblet-13) cells. Similarly, marker genes for basal (i.e., basal, basal-B) and ciliated subclusters (i.e., ciliated, ciliated-B) were defined in analogous manners. The color scale encodes the −log 10(P-value) from right-sided Fisher's exact test.

FIG. 22 is a dot plot depicting gene expression levels and percentage of cells expressing marker genes across each cell cluster.

FIG. 23A-FIG. 23C depict the construction of multi-modal Bayesian Networks. (A) Summary of asthma and control Bayesian Networks for nasal brushing. (B) Enrichment of asthma related signatures genes in nasal brushing asthma (y-axis) and control networks (x-axis). −log(P) from right-sided Fisher's exact test were shown. Orange indicates that each signature gene set is significant in both the asthma and control networks, while purple and green indicate significance exclusively in the asthma or control networks, respectively. (C) Enrichment of cell type marker genes in nasal brushing asthma (y-axis) and control networks (x-axis). The colors indicate the same information as described previously. The shapes indicate whether the cell type markers are exclusive (triangle) in comparison to other cell types or non-exclusive (circle).

FIG. 24 depicts benchmark comparisons of five deconvolution methodologies on the GSE136831 dataset with 78 samples. Pearson correlation (r) and root-mean-square error (RMSE) values between the cell type proportions computed by the different deconvolution methods and known compositions of pseudo-bulk mixtures are shown.

FIG. 25 depicts a block diagram of a system that is suitable for use in practicing various embodiments of the disclosure.

### DETAILED DESCRIPTION

Before the disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting, because the scope of the invention will be limited only by the appended claims.

Unless defined otherwise, all technical and scientific terms used herein have the same meaning as commonly understood by one of ordinary skill in the art to which this invention belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the terms “treat,” “treating,” or the like, mean to alleviate symptoms, eliminate the causation of symptoms either on a temporary or permanent basis, or to prevent or slow the appearance of symptoms of a particular disorder or condition, e.g., asthma.

Although any methods and materials similar or equivalent to those described herein can be used in the practice of the invention, the typical methods and materials are now described. All publications mentioned herein are incorporated herein by reference in their entirety.

In some embodiments, methods to treat or alleviate one or more conditions or complications associated with one or more chronic airway diseases are provided, wherein the method includes the use of a drug that promotes transdifferentiation of goblet cells into ciliated cells. In certain embodiments, the chronic airway disease includes, but is not limited to, asthma, chronic rhinosinusitis, allergic rhinitis, allergic fungal rhinosinusitis, chronic sinusitis, allergic bronchopulmonary aspergillosis (ABPA), bronchiectasis, unified airway disease, eosinophilic granulomatosis with polyangiitis (EGPA, formerly known as Churg-Strauss syndrome), gastroesophageal reflux disease (GERD), cystic fibrosis (CF), chronic obstructive pulmonary disease (COPD), eosinophilic esophagitis (EoE), chronic rhinosinusitis with nasal polyps (CRSwNP), chronic rhinosinusitis without nasal polyps (CRSsNP), aspirin hypersensitivity, NSAID exacerbated respiratory disease (NSAID-ERD), perennial allergic rhinitis (PAR), food allergy, chronic eosinophilic pneumonia (CEP), and exercise induced bronchospasm, and the like.

In some embodiments, methods to screen for a suitable treatment drug candidate for treating or alleviating one or more conditions or complications associated with one or more chronic airway diseases are provided, wherein the method includes determining whether the drug candidate promotes transdifferentiation of goblet cells into ciliated cells. In certain embodiments, the chronic airway disease includes, but is not limited to, asthma, chronic rhinosinusitis, allergic rhinitis, allergic fungal rhinosinusitis, chronic sinusitis, allergic bronchopulmonary aspergillosis (ABPA), bronchiectasis, unified airway disease, eosinophilic granulomatosis with polyangiitis (EGPA, formerly known as Churg-Strauss syndrome), gastroesophageal reflux disease (GERD), cystic fibrosis (CF), chronic obstructive pulmonary disease (COPD), eosinophilic esophagitis (EoE), chronic rhinosinusitis with nasal polyps (CRSwNP), chronic rhinosinusitis without nasal polyps (CRSsNP), aspirin hypersensitivity, NSAID exacerbated respiratory disease (NSAID-ERD), perennial allergic rhinitis (PAR), food allergy, chronic eosinophilic pneumonia (CEP), and exercise induced bronchospasm, and the like.

In some embodiments, a bioassay is provided to assess in a patient a drug candidate's ability to transform goblet cells to ciliated cells. In certain embodiments, the chronic airway disease includes, but is not limited to, asthma, chronic rhinosinusitis, allergic rhinitis, allergic fungal rhinosinusitis, chronic sinusitis, allergic bronchopulmonary aspergillosis (ABPA), unified airway disease, eosinophilic granulomatosis with polyangiitis (EGPA, formerly known as Churg-Strauss syndrome), gastroesophageal reflux disease (GERD), cystic fibrosis (CF), chronic obstructive pulmonary disease (COPD), eosinophilic esophagitis (EoE), chronic rhinosinusitis with nasal polyps (CRSwNP), chronic rhinosinusitis without nasal polyps (CRSsNP), aspirin hypersensitivity, NSAID exacerbated respiratory disease (NSAID-ERD), perennial allergic rhinitis (PAR), food allergy, chronic eosinophilic pneumonia (CEP), and exercise induced bronchospasm, and the like.

### Methods for Reducing the Incidence of Asthma Exacerbations

Methods for reducing the incidence of asthma exacerbations in a subject in need thereof comprising administering an asthma therapy to a subject with a marker profile described herein are provided. According to certain embodiments, the asthma therapy is treatment of the subject with a biologic compound, e.g., an antibody or antigen-binding fragment thereof. Exemplary biologic compounds that can be used in the context of the methods featured herein include, but are not limited to, dupilumab, benralizumab, mepolizumab, omalizumab, reslizumab, tezepelumab and the like, and antigen-binding fragments thereof.

In certain embodiments, a subject is selected for treatment with a biologic agent if the subject has an increase in ciliated cell gene signature expression levels relative to a control. In certain exemplary embodiments, the ciliated cell gene signature is an epithelial ciliated signature. In certain exemplary embodiments, the ciliated cell gene signature is an epithelial proximal ciliated signature.

In certain exemplary embodiments, a subject is selected for treatment with a biologic agent if the subject has an increase in one or more biomarkers, such as at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least eleven biomarkers, at least twelve biomarkers, at least thirteen biomarkers, at least fourteen biomarkers, at least fifteen biomarkers, at least sixteen biomarkers, at least seventeen biomarkers, at least eighteen biomarkers, or at least nineteen biomarkers, that are a gene selected from the group consisting of C20orf85, FAM92B, CAPS, MORN5, CAPSL, ZMYND10, SNTN, TCTEX1D2, PIFO, RSPH1, DYNLRB2, C2orf40, SPA17, CCDC170, C11orf88, CETN2, ROPN1L, CCDC78 and C9orf24. In certain exemplary embodiments, a subject is selected for treatment with a biologic agent if the subject has an increase in one or more biomarkers, such as at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least eleven biomarkers, at least twelve biomarkers, at least thirteen biomarkers, at least fourteen biomarkers, at least fifteen biomarkers, at least sixteen biomarkers, at least seventeen biomarkers, at least eighteen biomarkers, or at least nineteen biomarkers, that are a gene selected from the group consisting of C11orf88, C20orf85, C2orf40, C9orf24, CAPS, CAPSL, CCDC170, CCDC78, CETN2, DYNLRB2, FAM92B, MORN5, PIFO, ROPN1L, RSPH1, SNTN, SPA17, TCTEX1D2 and ZMYND10.

In certain exemplary embodiments, a subject is selected for treatment with a biologic agent if the subject has an increase in one or more biomarkers, such as at least one biomarker, at least two biomarkers, at least three biomarkers, at least four biomarkers, at least five biomarkers, or at least six biomarkers, that are from a ciliated cell gene signature that optionally comprises one or more genes selected from the group consisting of FAM92B, CAPSL, C20orf85, C11orf88, CAPS, and RSPH9. In certain exemplary embodiments, a subject is selected for treatment with a biologic agent if the subject has an increase in one or more biomarkers, such as at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least eleven biomarkers, at least twelve biomarkers, at least thirteen biomarkers, or at least fourteen biomarkers, that are from an epithelial ciliated gene signature that optionally comprises genes selected from the group consisting of FAM92B, CAPSL, C20orf85, C11orf88, SNTN, ROPN1L, ZMYND10, C9orf24, MORN5, CCDC78, CCDC170, RSPH9, C9orf116, and RSPH1. In certain exemplary embodiments, a subject is selected for treatment with a biologic agent if the subject has an increase in one or more biomarkers, such as at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least eleven biomarkers, at least twelve biomarkers, at least thirteen biomarkers, at least fourteen biomarkers, at least fifteen biomarkers, at least sixteen biomarkers, at least seventeen biomarkers, at least eighteen biomarkers, at least nineteen biomarkers, at least twenty biomarkers, at least twenty-one biomarkers, or at least twenty-two biomarkers, that are of an epithelial proximal ciliated gene signature that optionally comprises genes selected from the group consisting of CAPSL, C20orf85, SNTN, CAPS, ROPN1L, PIFO, ZMYND10, C9orf24, MORN5, C2orf40, WDR54, CRIP1, DYNLRB2, SPA17, CES1, C9orf116, RSPH1, TCTEX1D2, TSPAN1, CETN2, TUBB4B, and DYNLT1.

According to certain embodiments, a subject in need thereof has asthma that is controlled using inhaled asthma therapy. According to certain embodiments, a subject in need thereof has asthma that may be controlled using one or more biologic compounds. According to certain embodiments, a subject in need thereof has asthma that has low levels of one or more type 2 markers. According to certain embodiments, a subject in need thereof has asthma that failed to be controlled using two or more different biologics. According to certain embodiments, a subject in need thereof has unclassified asthma.

As used herein, “asthma exacerbation” means an increase in the severity and/or frequency and/or duration of one or more symptoms or indicia of asthma. An “asthma exacerbation” also includes any deterioration in the respiratory health of a subject that requires and or is treatable by a therapeutic intervention for asthma (such as, e.g., steroid treatment, inhaled corticosteroid treatment, hospitalization, etc.). There are two types of asthma exacerbation events: a loss of asthma control (LOAC) event and a severe exacerbation event.

According to certain embodiments, a loss of asthma control (LOAC) event is defined as one or more of the following: (a) greater than or equal to 6 additional reliever puffs of salbutamol/albuterol or levosalbutamol/levalbuterol in a 24 hour period (compared to baseline) on 2 consecutive days; (b) an increase in ICS greater than or equal to 4 times the dose at visit 2; and (c) use of systemic corticosteroids for greater than or equal to 3 days; or (d) hospitalization or emergency room visit because of asthma, requiring systemic corticosteroids.

In certain instances, an asthma exacerbation may be categorized as a “severe asthma exacerbation event.” A severe asthma exacerbation event means an incident requiring immediate intervention in the form of treatment with either systemic corticosteroids or with inhaled corticosteroids at four or more times the dose taken prior to the incident. According to certain embodiments, a severe asthma exacerbation event is defined as a deterioration of asthma requiring: use of systemic corticosteroids for greater than or equal to 3 days; or hospitalization or emergency room visit because of asthma, requiring systemic corticosteroids. The general expression “asthma exacerbation” therefore includes and encompasses the more specific subcategory of “severe asthma exacerbations.” Accordingly, methods for reducing the incidence of severe asthma exacerbations in a patient in need thereof are included.

A “reduction in the incidence” of an asthma exacerbation means that a subject who has received an asthma therapy experiences fewer asthma exacerbations (i.e., at least one fewer exacerbation) after treatment than before treatment, or experiences no asthma exacerbations for at least 4 weeks (e.g., 4, 6, 8, 12, 14, or more weeks) following initiation of treatment with the asthma therapy. A “reduction in the incidence” of an asthma exacerbation alternatively means that, following administration of the asthma therapy, the likelihood that a subject experiences an asthma exacerbation is decreased by at least 10% (e.g., 10%, 15%, 20%, 25%, 30%, 35%, 40%, 45%, 50%, or more) as compared to a subject who has not received the asthma therapy.

Methods for reducing the incidence of asthma exacerbations in a subject in need thereof comprising administering an asthma therapy comprising a biologic compound, e.g., an antibody or antigen-binding fragment thereof to the subject as well as administering to the subject one or more maintenance doses of an inhaled corticosteroid (ICS) and/or one or more maintenance doses of a second controller, e.g., a long-acting beta-agonist (LABA) or a leukotriene receptor antagonist (LTA), are provided.

Suitable biologic compounds include, but are not limited to, dupilumab, benralizumab, mepolizumab, omalizumab, reslizumab, tezepelumab, and the like, or antigen-binding fragments of any of these.

Suitable ICSs include, but are not limited to, fluticasone (e.g., fluticasone propionate, e.g., FLOVENT™), budesonide, mometasone (e.g., mometasone furoate, e.g., ASMANEX™) flunisolide (e.g., AEROBID™), dexamethasone acetate/phenobarbital/theophylline (e.g., AZMACORT™), beclomethasone dipropionate HFA (QVAR™), and the like.

Suitable LABAs include, but are not limited to, salmeterol (e.g., SEREVENT™) formoterol (e.g., FORADIL™), and the like.

Suitable LTAs include, but are not limited to, montelukast (e.g., SINGULAIR™) zafirlukast (e.g., Accolate™), and the like.

Methods for reducing the incidence of asthma exacerbations in a subject in need thereof comprising administering a pharmaceutical composition comprising an asthma therapy, e.g., a biologic compound, to the subject as well as administering to the subject one or more reliever medications to eliminate or reduce one or more asthma-associated symptoms, are provided. Suitable reliever medications include, but are not limited to, quick-acting beta2-adrenergic receptor agonists such as, e.g., albuterol (i.e., salbutamol, e.g., PROVENTIL™, VENTOLIN™ XOPENEX™ and the like), pirbuterol (e.g., MAXAIR™), metaproterenol (e.g., ALUPENT™) and the like.

### Methods for Improving Asthma-Associated Parameters

Methods for improving one or more asthma-associated parameters in a subject in need thereof, wherein the methods comprise administering a pharmaceutical composition comprising an asthma therapy, e.g., a biologic compound, are also provided. A reduction in the incidence of an asthma exacerbation (as described above) may correlate with an improvement in one or more asthma-associated parameters; however, such a correlation is not necessarily observed in all cases.

Examples of “asthma-associated parameters” include: (1) relative percent change from baseline (e.g., at week 12) in forced expiratory volume in 1 second (FEV₁); (2) a relative percent change from baseline (e.g., at week 12) as measured by forced expiratory flow at 25-75% of the pulmonary volume (FEF25-75%); (3) annualized rate of loss of asthma control events during the treatment period; (4) annualized rate of severe exacerbation events during the treatment period; (5) time to loss of asthma control events during the treatment period; (6) time to severe exacerbation events during the treatment period; (7) time to loss of asthma control events during overall study period; (8) time to severe exacerbation events during overall study period; (9) health care resource utilization; (10) change from baseline (e.g., at week 12) in: i) morning and evening asthma symptom scores, ii) ACQ-5 score, iii) AQLQ score, iv) morning and evening PEF, v) number of inhalations/day of salbutamol/albuterol or levosalbutamol/levalbuterol for symptom relief, vi) nocturnal awakenings; or (11) change from baseline (e.g., at week 12 or week 24) in: i) 22-item Sino Nasal Outcome Test (SNOT-22), ii) Hospital Anxiety and Depression Score (HADS), iii) EuroQual questionnaire (EQ-5D-3L or EQ-5D-5L). An “improvement in an asthma-associated parameter” means an increase from baseline of one or more of FEV₁, AM PEF or PM PEF, and/or a decrease from baseline of one or more of daily albuterol/levalbuterol use, ACQ5 score, average nighttime awakenings or SNOT-22 score. As used herein, the term “baseline,” with regard to an asthma-associated parameter, means the numerical value of the asthma-associated parameter for a patient prior to or at the time of administration of a pharmaceutical composition comprising an asthma therapy, e.g., a biologic compound.

To determine whether an asthma-associated parameter has “improved,” the parameter is quantified at baseline and at a time point after administration of the pharmaceutical composition described herein. For example, an asthma-associated parameter may be measured at day 1, day 2, day 3, day 4, day 5, day 6, day 7, day 8, day 9, day 10, day 11, day 12, day 14, or at week 3, week 4, week 5, week 6, week 7, week 8, week 9, week 10, week 11, week 12, week 13, week 14, week 15, week 16, week 17, week 18, week 19, week 20, week 21, week 22, week 23, week 24, or longer, after the initial treatment with the pharmaceutical composition. The difference between the value of the parameter at a particular time point following initiation of treatment and the value of the parameter at baseline is used to establish whether there has been an “improvement” in the asthma associated parameter (e.g., an increase or decrease, as the case may be, depending on the specific parameter being measured).

The terms “acquire” or “acquiring” as used herein, refer to obtaining possession of a physical entity, or a value, e.g., a numerical value, by “directly acquiring” or “indirectly acquiring” the physical entity or value, such as an asthma-associated parameter. “Directly acquiring” means performing a process (e.g., performing a synthetic or analytical method) to obtain the physical entity or value. “Indirectly acquiring” refers to receiving the physical entity or value from another party or source (e.g., a third-party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”).

Information that is acquired indirectly can be provided in the form of a report, e.g., supplied in paper or electronic form, such as from an online database or application (an “App”). The report or information can be provided by, for example, a healthcare institution, such as a hospital or clinic; or a healthcare provider, such as a doctor or nurse.

Forced Expiratory Volume in 1 Second (FEV₁). According to certain embodiments, administration of asthma therapy to a patient results in an increase from baseline of forced expiratory volume in 1 second (FEV₁). Methods for measuring FEV₁ are known in the art. For example, a spirometer that meets the 2005 American Thoracic Society (ATS)/European Respiratory Society (ERS) recommendations can be used to measure FEV₁ in a patient. The ATS/ERS Standardization of Spirometry may be used as a guideline. Spirometry is generally performed between 6 and 10 AM after an albuterol withhold of at least 6 hours. Pulmonary function tests are generally measured in the sitting position, and the highest measure is recorded for FEV₁ (in liters).

Therapeutic methods that result in an increase of FEV₁ from baseline of at least 0.05 L at week 12 following initiation of treatment with asthma therapy are provided. For example, administration of asthma therapy to a subject in need thereof causes an increase of FEV₁ from baseline of about 0.05 L, 0.10 L, 0.12 L, 0.14 L, 0.16 L, 0.18 L, 0.20 L, 0.22 L, 0.24 L, 0.26 L, 0.28 L, 0.30 L, 0.32 L, 0.34 L, 0.36 L, 0.38 L, 0.40 L, 0.42 L, 0.44 L, 0.46 L, 0.48 L, 0.50 L, or more at week 12.

FEF25-75%. According to certain embodiments, administration of asthma therapy to a patient results in an increase from baseline of FEF25-75%. Methods for measuring FEF are known in the art. For example, a spirometer that meets the 2005 American Thoracic Society (ATS)/European Respiratory Society (ERS) recommendations can be used to measure FEV₁ in a patient. The FEF25-75% (forced expiratory flow between 25% and 75%) is the speed (in liters per second) at which a person can empty the middle half of his or her air during a maximum expiration (i.e., Forced Vital Capacity or FVC). The parameter relates to the average flow from the point at which 25 percent of the FVC has been exhaled to the point at which 75 percent of the FVC has been exhaled. The FEF25-75% of a subject provides information regarding small airway function, such as the extent of small airway disease and/or inflammation. A change in FEF25-75% is an early indicator of obstructive lung disease. In certain embodiments, an improvement and/or increase in the FEF25-75% parameter is an improvement of at least 10%, 25%, 50% or more as compared to baseline. In certain embodiments, the methods described herein result in normal FEF25-75% values in a subject (e.g., values ranging from 50-60% and up to 130% of the average).

Morning and Evening Peak Expiratory Flow (AM PEF and PM PEF). According to certain embodiments, administration of asthma therapy to a patient results in an increase from baseline of morning (AM) and/or evening (PM) peak expiratory flow (AM PEF and/or PM PEF). Methods for measuring PEF are known in the art. For example, according to one method for measuring PEF, patients are issued an electronic PEF meter for recording morning (AM) and evening (PM) PEF (as well as daily albuterol use, morning and evening asthma symptom scores, and number of nighttime awakenings due to asthma symptoms that require rescue medications). Patients are instructed on the use of the device, and written instructions on the use of the electronic PEF meter are provided to the patients. In addition, a medical professional may instruct the patients on how to record pertinent variables in the electronic PEF meter. AM PEF is generally performed within 15 minutes after arising (between 6 am and 10 am) prior to taking any albuterol. PM PEF is generally performed in the evening (between 6 pm and 10 pm) prior to taking any albuterol. Subjects should try to withhold albuterol for at least 6 hours prior to measuring their PEF. Three PEF efforts are performed by the patient and all 3 values are recorded by the electronic PEF meter. Usually the highest value is used for evaluation. Baseline AM PEF may be calculated as the mean AM measurement recorded for the 7 days prior to administration of the first dose of a biologic compound, and baseline PM PEF may be calculated as the mean PM measurement recorded for the 7 days prior to administration of the first dose of pharmaceutical composition comprising a biologic compound.

Therapeutic methods that result in an increase in AM PEF and/or PM PEF from baseline of at least 1.0 L/min at week 12 following initiation of treatment with asthma therapy are provided. For example, according to exemplary embodiments, administration of a biologic compound to a subject in need thereof causes an increase in PEF from baseline of about 0.5 L/min, 1.0 L/min, 1.5 L/min, 2.0 L/min, 2.5 L/min, 3.0 L/min, 3.5 L/min, 4.0 L/min, 4.5 L/min, 5.0 L/min, 5.5 L/min, 6.0 L/min, 6.5 L/min, 7.0 L/min, 7.5 L/min, 8.0 L/min, 8.5 L/min, 9.0 L/min, 9.5 L/min, 10.0 L/min, 10.5 L/min, 11.0 L/min, 12.0 L/min, 15 L/min, 20 L/min, or more at week 12.

Albuterol/Levalbuterol Use. According to certain embodiments, administration asthma therapy to a patient results in a decrease from baseline of daily albuterol or levalbuterol use. The number of albuterol/levalbuterol inhalations can be recorded daily by the patients in a diary, PEF meter, or other recording device. During treatment with the pharmaceutical composition described herein, use of albuterol/levalbuterol typically may be on an as-needed basis for symptoms, not on a regular basis or prophylactically. The baseline number of albuterol/levalbuterol inhalations/day may be calculated based on the mean for the 7 days prior to administration of the first dose of an asthma therapy.

Therapeutic methods are provided that result in a decrease in albuterol/levalbuterol use from baseline of at least 0.25 puffs per day at week 12 following initiation of treatment with a pharmaceutical composition comprising a biologic compound. For example, administration of an asthma therapy to a subject in need thereof causes a decrease in albuterol/levalbuterol use from baseline of about 0.25 puffs per day, 0.50 puffs per day, 0.75 puffs per day, 1.00 puff per day, 1.25 puffs per day, 1.5 puffs per day, 1.75 puffs per day, 2.00 puffs per day, 2.25 puffs per day, 2.5 puffs per day, 2.75 puffs per day, 3.00 puffs per day, or more at week 12.

OCS Use. According to certain embodiments, administration of an asthma therapy to a patient can be used in conjunction with an OCS such as oral prednisone. The number of OCS administrations can be recorded daily by the patients in a diary, PEF meter, or other recording device. During treatment with the asthma therapy described herein, occasional short-term use of prednisone typically can be used to control acute asthmatic episodes, e.g., episodes in which bronchodilators and other anti-inflammatory agents fail to control symptoms. In other aspects, prednisone is used concurrent with or as a substitution for ICS. Oral prednisone may be administered in dosages of about 5 mg, 10 mg, 15 mg, 20 mg, 25 mg, 30 mg, 35 mg or 40 mg. OCS can optionally be administered once a day or multiple times a day (e.g., twice a day, three times a day, four times a day, etc.)

In certain exemplary embodiments, methods for reducing or eliminating the dependency of the subject on OCS use are provided. The reduction or elimination of steroid dependency is highly advantageous and desirable. In certain embodiments, a reduction of 50% or greater (e.g., 50%, 60%, 70%, 80%, 90% or more) in the OCS dose is achieved after administration of asthma therapy at a period of time (e.g., at week 24). In certain embodiments, the OCS is substantially eliminated after 40 weeks, 45 weeks, 50 weeks, 52 weeks, or greater after first dose following administration of an initial dose of an asthma therapy. In other embodiments, the level of OCS use is reduced to less than 5 mg per day (e.g., less than 5 mg, 4 mg, 3 mg, 2 mg or less per day). In other embodiments, the dependency on OCS use is substantially eliminated after 3 months, 6 months, 9 months or 1 year following treatment an asthma therapy.

5-Item Asthma Control Questionnaire (ACQ) Score. According to certain embodiments, administration of an asthma therapy to a patient results in a decrease from baseline of five-item Asthma Control Questionnaire (ACQ5) score. The ACQ5 is a validated questionnaire to evaluate asthma control.

Therapeutic methods are provided that result in a decrease in ACQ5 score from baseline of at least 0.10 points at week 12 following initiation of treatment with a pharmaceutical composition comprising an asthma therapy. For example, administration of an asthma therapy to a subject in need thereof causes a decrease in ACQ score from baseline of about 0.10 points, 0.15 points, 0.20 points, 0.25 points, 0.30 points, 0.35 points, 0.40 points, 0.45 points, 0.50 points, 0.55 points, 0.60 points, 0.65 points, 0.70 points, 0.75 points, 0.80 points, 0.85 points, or more at week 12.

Night-Time Awakenings. According to certain embodiments, administration of an asthma therapy to a patient results in a decrease from baseline of average number of nighttime awakenings.

In certain embodiments, the methods decrease the average number of nighttime awakenings from baseline by at least about 0.10 times per night at week 12 following initiation of treatment. For example, administration of an asthma therapy to a subject in need thereof can cause a decrease in average number of nighttime awakenings from baseline of about 0.10 times per night, 0.15 times per night, 0.20 times per night, 0.25 times per night, 0.30 times per night, 0.35 times per night, 0.40 times per night, 0.45 times per night, 0.50 times per night, 0.55 times per night, 0.60 times per night, 0.65 times per night, 0.70 times per night, 0.75 times per night, 0.80 times per night, 0.85 times per night, 0.90 times per night, 0.95 times per night, 1.0 times per night, 2.0 times per night, or more at week 12.

22-Item Sinonasal Outcome Test (SNOT-22) Score. According to certain embodiments, administration of an asthma therapy to a patient results in a decrease from baseline of 22-item Sinonasal Outcome Test (SNOT-22). The SNOT-22 is a validated questionnaire to assess the impact of chronic rhinosinusitis on quality of life (Hopkins et al 2009, Clin. Otolaryngol. 34: 447-454).

Therapeutic methods are provided that result in a decrease in SNOT-22 score from baseline of at least 1 point at week 12 following initiation of treatment with a pharmaceutical composition comprising an asthma therapy. For example, administration of a biologic compound to a subject in need thereof can cause a decrease in SNOT-22 score from baseline of about 1, 2, 3, 4, 5, 6, 7, 8, 9, 10, 11, 12, 13 points, or more at week 12.

### Biomarkers

In certain embodiments, the subject experiences an improvement in lung function as measured by a biomarker, e.g., a biomarker associated with asthma. In certain exemplary embodiments, a biomarker may be selected from the group consisting of fractional exhaled nitric oxide (FeNO), eotaxin-3, total IgE, allergen-specific IgE, periostin, eosinophil (Eos) level, and thymus and activation-regulated chemokine (TARC). In certain embodiments, an improvement in lung function is indicated by a reduction or an increase (as appropriate) at week 4, week 12 or week 24 following treatment.

### Methods for Treating Asthma

In some embodiments, methods are provided for treating asthma, moderate-to-severe uncontrolled asthma or inadequately controlled asthma, as well as allergic forms of any of these, in a subject in need thereof, wherein the methods comprise administering a pharmaceutical composition comprising a biologic compound to the subject.

As used herein, the term “asthma” can be used interchangeably with “intermittent asthma,” or “bronchial asthma.” “Asthma,” “bronchial asthma” and “intermittent asthma,” and allergic forms of each of these, refer to asthma in which one or any combination of the following are true: symptoms occur 2 or fewer days per week; symptoms do not interfere with normal activities; nighttime symptoms occur fewer than 2 days per month; or one or more lung function tests (e.g., forced expiratory volume in one second (FEV₁) and/or peak expiratory flow (PEF) of greater than 80%) are normal when the subject is not suffering from an asthma attack.

“Allergic asthma” refers to asthma that is triggered by allergens, e.g., inhaled allergens, such as dust mites, pet dander, pollen, fungi and the like. As used herein, the term “allergic asthma” refers to asthma in combination with one or more allergic markers, e.g., total serum IgE (e.g., a total serum IgE of ≥30 IU/mL, a total serum IgE of ≥700 IU/mL, or total serum IgE of ≥1000 IU/mL), and/or at least one positive allergen-specific IgE value (e.g., an allergen-specific IgE value of ≥0.35 kU/L). In certain embodiments, the allergen is an airborne aeroallergen (e.g., an annual aeroallergen or a perennial aeroallergen).

As used herein, the term “persistent asthma” or “persistent bronchial asthma” refers to asthma that is more severe than (bronchial) asthma/intermittent (bronchial) asthma. A subject suffering from persistent asthma or persistent bronchial asthma experiences one or more of the following: symptoms more than 2 days per week; symptoms that interfere with normal activities; nighttime symptoms that occur more than 2 days per month; or one or more lung function tests (e.g., forced expiratory volume in one second (FEV₁) and/or peak expiratory flow (PEF) of less than 80%) that are not normal when the subject is not suffering from an asthma attack; the subject relies on daily asthma control medication; the subject has taken a systemic steroid more than once in the last year after a severe asthma flare-up; or use of a short-acting beta-2 agonist more than two days per week for relief of asthma symptoms.

Asthma/intermittent asthma, bronchial asthma/intermittent bronchial asthma, and persistent asthma/persistent bronchial asthma, and allergic forms of each of these, can be categorized as “mild,” “moderate,” “severe” or “moderate-to-severe.” “Mild intermittent asthma” or “mild intermittent bronchial asthma” is defined as having symptoms less than once a week, and having forced expiratory volume in one second (FEV₁) or peak expiratory flow (PEF) ≥80%. “Mild persistent asthma” or “mild persistent bronchial asthma” differs in that symptoms frequency is greater than once per week but less than once per day, and variability in FEV₁ or PEF is <20%-30%. “Moderate intermittent asthma” or “moderate intermittent bronchial asthma” is defined as having symptoms less than once a week, and having forced expiratory volume in one second (FEV₁) or peak expiratory flow (PEF) of 60-80%. “Moderate persistent asthma” or “moderate persistent bronchial asthma,” or an allergic form thereof, is defined as having daily symptoms, exacerbations that may affect activity and/or sleep, nocturnal symptoms more than once a week, daily use of inhaled short-acting beta-2 agonist and having forced expiratory volume in one second (FEV₁) or peak expiratory flow (PEF) of 60-80%. “Severe intermittent asthma” or “severe intermittent bronchial asthma,” or an allergic form thereof, is defined as having symptoms less than once a week, and having forced expiratory volume in one second (FEV₁) or peak expiratory flow (PEF) of 60%. “Severe persistent asthma” or “severe persistent bronchial asthma” is defined as having daily symptoms, frequent exacerbations that may affect activity and/or sleep, frequent nocturnal symptoms, limitation of physical activities, daily use of inhaled short-acting beta-2 agonist, and having forced expiratory volume in one second (FEV₁) or peak expiratory flow (PEF) of 60%. “Moderate-to-severe intermittent asthma” or “moderate-to-severe intermittent bronchial asthma,” or an allergic form thereof, is defined as having symptoms between those of moderate intermittent asthma/moderate intermittent bronchial asthma and severe intermittent asthma/severe intermittent bronchial asthma. “Moderate-to-severe persistent asthma” or “moderate-to-severe persistent bronchial asthma,” or an allergic form thereof, is defined as having symptoms between those of moderate persistent asthma/moderate persistent bronchial asthma and severe persistent asthma/severe persistent bronchial asthma.

As used herein, the term “inadequately controlled asthma,” or an allergic form thereof, refers to patients whose asthma is either “not well controlled” or “very poorly controlled” as defined by the “Expert Panel Report 3: Guidelines for the Diagnosis and Management of Asthma,” National Heart, Blood and Lung Institute, NIH, Aug. 28, 2007. “Not well controlled asthma,” or an allergic form thereof, is defined as having symptoms greater than two days per week, nighttime awakenings one to three times per week, some limitations on normal activity, short-acting beta2-agonist use for symptom control greater than two days per week, FEV₁ of 60-80% of predicted and/or personal best, an ATAQ score of 1-2, an ACQ score of 1.5 or greater, and an ACT score of 16-19. “Very poorly controlled asthma,” or an allergic form thereof, is defined as having symptoms throughout the day, nighttime awakenings four times or more per week, extreme limitations on normal activity, short-acting beta2-agonist use for symptom control several times per day, FEV₁ of less than 60% of predicted and/or personal best, an ATAQ score of 3-4, an ACQ score of N/A, and an ACT score of less than or equal to 15.

In some embodiments, a subject is identified as having “moderate-to-severe uncontrolled” asthma if the subject receives such a diagnosis from a physician, based on the Global Initiative for Asthma (GINA) 2009 Guidelines, and one or more of the following criteria: i) Existing treatment with moderate- or high-dose ICS/LABA (2 fluticasone propionate 250 μg twice daily or equipotent ICS daily dosage) with a stable dose of ICS/LABA for greater than or equal to 1 month prior to administration of an initial dose of an asthma therapy; ii) FEV₁ 40 to 80% predicted normal prior to administration of an initial dose of an asthma therapy; iii) ACQ-5 score greater than or equal to 1.5 prior to administration of an initial dose of an asthma therapy; iv) reversibility of at least 12% and 200 mL in FEV₁ after 200 μg to 400 μg (2 to 4 inhalations) of salbutamol/albuterol prior to administration of an initial dose of a biologic compound; or v) has experienced, within 1 year prior to administration of an initial dose of an asthma therapy, any of the following events: (a) treatment with greater than or equal to 1 systemic (oral or parenteral) steroid burst for worsening asthma, (b) hospitalization or an emergency/urgent medical care visit for worsening asthma.

“Severe asthma” or “severe allergic asthma” refers to asthma in which adequate control cannot be achieved by high-dose treatment with inhaled corticosteroids and additional controllers (e.g., long-acting inhaled beta 2 agonists, montelukast, and/or theophylline) or by oral corticosteroid treatment (e.g., for at least six months per year), or is lost when the treatment is reduced. In certain embodiments, severe asthma includes asthma that is treated with high-dose ICS and at least one additional controller (e.g., LABA, montelukast, or theophylline) or oral corticosteroids >6 months/year, wherein at least one of the following occurs or would occur if treatment is reduced: ACT <20 or ACQ >1.5; at least 2 exacerbations in the last 12 months; at least 1 exacerbation treated in hospital or requiring mechanical ventilation in the last 12 months; or FEV₁<80% (if FEV₁/FVC below the lower limit of normal).

“Steroid-dependent asthma” or “steroid-dependent allergic asthma” refers to asthma which requires one or more of the following treatments: frequent, short term oral corticosteroid treatment bursts in the past 12 months; regular use of high dose inhaled corticosteroids in the past 12 months; regular use of injected long acting corticosteroids; daily use of oral corticosteroids; alternate-day oral corticosteroids; or prolonged use of oral corticosteroids in the past year.

“Oral corticosteroid-dependent asthma” of “oral corticosteroid-dependent allergic asthma” refers to a subject having ≥3 30-day oral corticosteroid (OCS) fills over a 12-month period and a primary asthma diagnosis within 12 months of the first OCS fill. Subjects with OCS-dependent asthma may also experience one or any combination of the following: have received physician prescribed LABA and high dose ICS (total daily dose >500 g fluticasone propionate dry powder formulation equivalent) for at least 3 months (the ICS and LABA can be parts of a combination product, or given by separate inhalers); have received additional maintenance asthma controller medications according to standard practice of care e.g., leukotriene receptor antagonists (LTRAs), theophylline, long-acting muscarinic antagonists (LAMAs), secondary ICS and cromones; received OCS for the treatment of asthma at a dose of between ≥7.5 to ≤30 mg (prednisone or prednisolone equivalent); have received an OCS dose administered every other day (or different doses every other day); morning pre-bronchodilator (BD) FEV₁ of <80% predicted normal; have evidence of asthma as documented by post-BD (albuterol/salbutamol) reversibility of FEV₁≥12% and >200 mL (15-30 min after administration of 4 puffs of albuterol/salbutamol); or have a history of at least one asthma exacerbation event within 12 months.

In one aspect, methods for treating asthma are provided comprising: (a) selecting a patient that exhibits wherein an increase in expression level of at least one gene, at least two genes, at least three genes, at least four genes, at least five genes, at least six genes, at least seven genes, at least eight genes, at least nine genes, at least ten genes, at least eleven genes, at least twelve genes, at least thirteen genes, at least fourteen genes, at least fifteen genes, at least sixteen genes, at least seventeen genes, at least eighteen genes, or at least nineteen genes selected from the group consisting of C20orf85, FAM92B, CAPS, MORN5, CAPSL, ZMYND10, SNTN, TCTEX1D2, PIFO, RSPH1, DYNLRB2, C2orf40, SPA17, CCDC170, C11orf88, CETN2, ROPN1L, CCDC78 and C9orf24 relative to a control; and (b) administering to the patient a pharmaceutical composition comprising an asthma therapy, e.g., a biologic compound optionally selected from dupilumab, benralizumab, mepolizumab, omalizumab, reslizumab and tezepelumab. In some embodiments,

In one aspect, methods for treating asthma are provided comprising: (a) selecting a patient that exhibits an increased expression level of at least one gene, at least two genes, at least three genes, at least four genes, at least five genes, at least six genes, at least seven genes, at least eight genes, at least nine genes, at least ten genes, at least eleven genes, at least twelve genes, at least thirteen genes, at least fourteen genes, at least fifteen genes, at least sixteen genes, at least seventeen genes, at least eighteen genes, or at least nineteen genes selected from the group consisting of C11orf88, C20orf85, C2orf40, C9orf24, CAPS, CAPSL, CCDC170, CCDC78, CETN2, DYNLRB2, FAM92B, MORN5, PIFO, ROPN1L, RSPH1, SNTN, SPA17, TCTEX1D2 and ZMYND10; and (b) administering to the patient a pharmaceutical composition comprising an asthma therapy, e.g., a biologic compound optionally selected from dupilumab, benralizumab, mepolizumab, omalizumab, reslizumab and tezepelumab.

In one aspect, methods for treating asthma are provided comprising: (a) selecting a patient that exhibits an increase in the ciliated cell gene signature expression levels observed relative to a control; and (b) administering to the patient a pharmaceutical composition comprising an asthma therapy, e.g., a biologic compound optionally selected from dupilumab, benralizumab, mepolizumab, omalizumab, reslizumab and tezepelumab. In certain exemplary embodiments, the ciliated cell gene signature comprises at least one gene, at least two genes, at least three genes, at least four genes, at least five genes, or at least six genes selected from the group consisting of FAM92B, CAPSL, C20orf85, C11orf88, CAPS and RSPH9. In certain exemplary embodiments, the ciliated cell gene signature is an epithelial ciliated signature. In certain exemplary embodiments, the epithelial ciliated signature comprises at least one gene, at least two genes, at least three genes, at least four genes, at least five genes, at least six genes, at least seven genes, at least eight genes, at least nine genes, at least ten genes, at least eleven genes, at least twelve genes, at least thirteen genes, at least fourteen genes selected from the group consisting of FAM92B, CAPSL, C20orf85, C11orf88, SNTN, ROPN1L, ZMYND10, C9orf24, MORN5, CCDC78, CCDC170, RSPH9, C9orf116 and RSPH1. In certain exemplary embodiments, the ciliated cell gene signature is an epithelial proximal ciliated signature. In certain exemplary embodiments, the epithelial proximal ciliated signature comprises at least one gene, at least two genes, at least three genes, at least four genes, at least five genes, at least six genes, at least seven genes, at least eight genes, at least nine genes, at least ten genes, at least eleven genes, at least twelve genes, at least thirteen genes, at least fourteen genes, at least fifteen genes, at least sixteen genes, at least seventeen genes, at least eighteen genes, at least nineteen genes, at least twenty genes, at least twenty-one genes, or at least twenty-two genes selected from the group consisting of CAPSL, C20orf85, SNTN, CAPS, ROPN1L, PIFO, ZMYND10, C9orf24, MORN5, C2orf40, WDR54, CRIP1, DYNLRB2, SPA17, CES1, C9orf116, RSPH1, TCTEX1D2, TSPAN1, CETN2, TUBB4B and DYNLT1.

### Biologic Compounds

The methods featured herein comprise administering to a subject in need thereof a therapeutic composition comprising a biologic compound. In certain exemplary embodiments, a biologic compound comprises an antibody selected from the group consisting of dupilumab, benralizumab, mepolizumab, omalizumab, reslizumab, and tezepelumab, as well as antigen-binding fragments of any of these.

Dupilumab is also known as DUPIXENT® and is commercially available from Sanofi and Regeneron Pharmaceuticals, Inc. Dupilumab is an anti-IL-4Ra monoclonal antibody that inhibits signaling of both IL-4 and IL-13.

Benralizumab is also known as FASENRA® and is commercially available from AstraZeneca. Benralizumab is an anti-IL-5 monoclonal antibody.

Mepolizumab is also known as NUCALA® and is commercially available from GSK. Mepolizumab is an anti-IL-5 monoclonal antibody.

Omalizumab is also known as XOLAIR® and is commercially available from Genentech. Omalizumab is an anti-IgE monoclonal antibody.

Reslizumab is also known as CINQAIR® and is commercially available from Teva. Reslizumab is an anti-IL-5 monoclonal antibody.

Tezepelumab is also known as TEZSPIRE® and is commercially available from Amgen/AstraZeneca. Tezepelumab is an anti-thymic stromal lymphopoietin (TSLP) monoclonal antibody.

The term “antibody” refers to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2, and C_{H}3. Each light chain comprises a light chain variable region (abbreviated herein as LCVR or V_{L}) and a light chain constant region. The light chain constant region comprises one domain (C_{L}I). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments, the FRs of the anti-IL-4R antibody (or antigen-binding portion thereof) may be identical to the human germline sequences, or may be naturally or artificially modified. An amino acid consensus sequence may be defined based on a side-by-side analysis of two or more CDRs.

The term “antibody” also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds to an antigen to form a complex. Antigen-binding fragments of an antibody may be derived, e.g., from full antibody molecules using any suitable standard techniques, such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment.”

### Pharmaceutical Compositions

Methods that comprise administering an asthma therapy, e.g., a biologic compound, to a patient, wherein the asthma therapy is contained within a pharmaceutical composition are provided. The pharmaceutical compositions described herein are formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J. Pharm. Sci. Technol. 52:238-311.

The dose of an asthma therapy, e.g., a biologic compound, administered to a patient may vary depending upon the age and the size of the patient, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions comprising an asthma therapy, e.g., a biologic compound, may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical compositions described herein, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, intra-tracheal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition described herein can be delivered subcutaneously or intravenously with a standard needle and syringe. In addition, with respect to subcutaneous delivery, a pen delivery device (e.g., an autoinjector pen) readily has applications in delivering a pharmaceutical composition described herein. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™ OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition described herein include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park IL), to name only a few. Examples of large-volume delivery devices (e.g., large-volume injectors) include, but are not limited to, bolus injectors such as, e.g., BD Libertas West SmartDose, Enable Injections, SteadyMed PatchPump, Sensile SenseTrial, YPsomed YpsoDose, Bespak Lapas, and the like.

For direct administration to the sinuses, the pharmaceutical compositions described herein may be administered using, e.g., a microcatheter (e.g., an endoscope and microcatheter), an aerosolizer, a powder dispenser, a nebulizer or an inhaler. The methods include administration of an asthma therapy, e.g., a biologic compound, to a subject in need thereof, in an aerosolized formulation. For example, aerosolized antibodies to IL-4R may be administered to treat asthma in a patient. Aerosolized antibodies can be prepared as described in, for example, U.S. Pat. No. 8,178,098, incorporated herein by reference in its entirety.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant (e.g., polysorbate 80, HCO-50 (polyoxyethylene (50 mol) adduct of hydrogenated castor oil)), etc. As the oily medium, there are employed, e.g., sesame oil, soybean oil, etc., which may be used in combination with a solubilizing agent such as benzyl benzoate, benzyl alcohol, etc. The injection thus prepared is typically filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

### Dosage

The amount of a biologic compound comprising an antibody administered to a subject according to the methods described herein is, generally, a therapeutically effective amount. As used herein, the phrase “therapeutically effective amount” means an amount of a biologic compound comprising that results in one or more of: (a) a reduction in the incidence of asthma exacerbations; (b) an improvement in one or more asthma-associated parameters (as defined elsewhere herein); and/or (c) a detectable improvement in one or more symptoms or indicia of an upper airway inflammatory condition. A “therapeutically effective amount” also includes an amount of a biologic compound comprising that inhibits, prevents, lessens, or delays the progression of asthma in a subject.

In the case of a biologic compound comprising an antibody, a therapeutically effective amount can be from about 0.05 mg to about 700 mg, e.g., about 0.05 mg, about 0.1 mg, about 1.0 mg, about 1.5 mg, about 2.0 mg, about 3.0 mg, about 5.0 mg, about 7.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, or about 700 mg of the anti-IL-4R antibody. In certain embodiments, 300 mg of an anti-IL-4R antibody is administered.

The amount of a biologic compound comprising an antibody contained within the individual doses may be expressed in terms of milligrams of antibody per kilogram of patient body weight (i.e., mg/kg). For example, a biologic compound comprising an antibody may be administered to a patient at a dose of about 0.0001 to about 10 mg/kg of patient body weight. For example, a biologic compound comprising an antibody can be administered at a dose of 1 mg/kg, 2 mg/kg, 3 mg/kg, 4 mg/kg, 5 mg/kg or 6 mg/kg.

### Administration Regimens

According to certain embodiments, multiple doses of a biologic compound may be administered to a subject over a defined time course. Such methods comprise sequentially administering to a subject multiple doses of a biologic compound. As used herein, “sequentially administering” means that each dose of a biologic compound is administered to the subject at a different point in time, e.g., on different days separated by a predetermined interval (e.g., hours, days, weeks, or months). Methods that comprise sequentially administering to the patient a single initial dose of a biologic compound, followed by one or more secondary doses of the biologic compound, and optionally followed by one or more tertiary doses of the biologic compound, are provided.

Methods comprising administering to a subject a pharmaceutical composition comprising a biologic compound antagonist at a dosing frequency of about four times a week, twice a week, once a week (q1w), once every two weeks (every two weeks is used interchangeably with every other week, bi-weekly or q2w), once every three weeks (tri-weekly or q3w), once every four weeks (monthly or q4w), once every five weeks (q5w), once every six weeks (q6w), once every eight weeks (q8w), once every twelve weeks (q12w), or less frequently so long as a therapeutic response is achieved, are provided. In certain embodiments involving the administration of a pharmaceutical composition comprising a biologic compound, once a week dosing of an amount of about 75 mg, 100 mg, 150 mg, 200 mg, or 300 mg, can be employed. In other embodiments involving the administration of a pharmaceutical composition comprising a biologic compound, once every two weeks dosing (every two weeks is used interchangeably with every other week, bi-weekly or q2w) of an amount of about 75 mg, 100 mg, 150 mg, 200 mg, or 300 mg, can be employed. In other embodiments involving the administration of a pharmaceutical composition comprising a biologic compound, once every three weeks dosing of an amount of about 75 mg, 100 mg, 150 mg, 200 mg, or 300 mg, can be employed. In other embodiments involving the administration of a pharmaceutical composition comprising a biologic compound, once every four weeks dosing (monthly dosing) of an amount of about 75 mg, 100 mg, 150 mg, 200 mg, or 300 mg, can be employed. In other embodiments involving the administration of a pharmaceutical composition comprising a biologic compound, once every five weeks dosing of an amount of about 75 mg, 100 mg, 150 mg, 200 mg, or 300 mg, can be employed. In other embodiments involving the administration of a pharmaceutical composition comprising a biologic compound, once every six weeks dosing of an amount of about 75 mg, 100 mg, 150 mg, 200 mg, or 300 mg, can be employed. In other embodiments involving the administration of a biologic compound, once every eight weeks dosing of an amount of about 75 mg, 100 mg, 150 mg, 200 mg, or 300 mg, can be employed. In other embodiments involving the administration of a pharmaceutical composition comprising a biologic compound, once every twelve weeks dosing of an amount of about 75 mg, 100 mg, 150 mg, 200 mg, or 300 mg, can be employed. In one embodiment, the route of administration is subcutaneous.

The term “week” or “weeks” refers to a period of (n×7 days) ±2 days, e.g. (n×7 days) ±1 day, or (n×7 days), wherein “n” designates the number of weeks, e.g. 1, 2, 3, 4, 5, 6, 8, 12 or more.

The terms “initial dose,” “secondary doses,” and “tertiary doses,” refer to the temporal sequence of administration of a biologic compound. Thus, the “initial dose” is the dose that is administered at the beginning of the treatment regimen (also referred to as the “baseline dose”); the “secondary doses” are the doses that are administered after the initial dose; and the “tertiary doses” are the doses that are administered after the secondary doses. The initial, secondary, and tertiary doses may all contain the same amount of the biologic compound, but generally may differ from one another in terms of frequency of administration. In certain embodiments, however, the amount of a biologic compound contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” or “initial doses” followed by subsequent doses that are administered on a less frequent basis (e.g., “maintenance doses”). In one embodiment, the maintenance dose may be lower than the initial dose. For example, one or more initial doses of 600 mg of a biologic compound may be administered followed by maintenance doses of about 7 5 mg to about 300 mg.

In one exemplary embodiment, each secondary and/or tertiary dose is administered 1 to 14 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose” means, in a sequence of multiple administrations, the dose of a biologic compound that is administered to a patient prior to the administration of the very next dose in the sequence with no intervening doses.

The methods may include administering to a patient any number of secondary and/or tertiary doses of a biologic compound. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

Methods comprising sequential administration of a biologic compound and a second therapeutic agent, to a patient to treat asthma or an associated condition are provided. In some embodiments, the methods comprise administering one or more doses of a biologic compound followed by one or more doses (e.g., 2, 3, 4, 5, 6, 7, 8, or more) of a second therapeutic agent. For example, one or more doses of about 75 mg to about 300 mg of a biologic compound may be administered after which one or more doses (e.g., 2, 3, 4, 5, 6, 7, 8, or more) of a second therapeutic agent (e.g., an inhaled corticosteroid or a beta2-agonist or any other therapeutic agent, as described elsewhere herein) may be administered to treat, alleviate, reduce or ameliorate one or more symptoms of asthma. In some embodiments, a biologic compound is administered at one or more doses (e.g., 2, 3, 4, 5, 6, 7, 8, or more) resulting in an improvement in one or more asthma-associated parameters followed by the administration of a second therapeutic agent to prevent recurrence of at least one symptom of asthma. Alternative embodiments pertain to concomitant administration of a biologic compound and a second therapeutic agent. For example, one or more doses (e.g., 2, 3, 4, 5, 6, 7, 8, or more) of a biologic compound are administered and a second therapeutic agent is administered at a separate dosage at a similar or different frequency relative to the biologic compound antagonist. In some embodiments, the second therapeutic agent is administered before, after or concurrently with a biologic compound.

In certain embodiments, the IL-4R antagonist is administered every other week for 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks or more. In other embodiments, the IL-4R antagonist is administered every four weeks for 12 weeks, 16 weeks, 20 weeks, 24 weeks, 28 weeks, 32 weeks, 36 weeks, 40 weeks, 44 weeks, 48 weeks or more. In specific embodiments, the biologic compound is administered for at least 24 weeks.

The present disclosure is further illustrated by the following example which should not be construed as further limiting. The contents of the figures and all references, patents and published patent applications cited throughout this application are expressly incorporated herein by reference for all purposes.

Furthermore, in accordance with the present invention there may be employed conventional molecular biology, microbiology, and recombinant DNA techniques within the skill of the art. Such techniques are explained fully in the literature. See, e.g., Green & Sambrook, Molecular Cloning: A Laboratory Manual, Fourth Edition (2012) Cold Spring Harbor Laboratory Press, Cold Spring Harbor, New York; DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

### REFERENCES

Each reference is incorporated herein in its entirety for all purposes.
Tsai, Y. H., Parker, J. S., Yang, I. V. & Kelada, S. N. P. Meta-analysis of airway epithelium gene expression in asthma. Eur Respir J 51(2018).
Langfelder, P. & Horvath, S. WGCNA: an R package for weighted correlation network analysis. BMC Bioinformatics 9, 559 (2008).
Zhu, J., et al. Stitching together multiple data dimensions reveals interacting metabolomic and transcriptomic networks that modulate cell regulation. PLoS Biol 10, e1001301 (2012).
Alladina, J., et al. A human model of asthma exacerbation reveals transcriptional programs and cell circuits specific to allergic asthma. Sci Immunol 8, eabg6352 (2023).
Vieira Braga, F. A., et al. A cellular census of human lungs identifies novel cell states in health and in asthma. Nat Med 25, 1153-1163 (2019).
Jackson, N. D., et al. Single-Cell and Population Transcriptomics Reveal Pan-epithelial Remodeling in Type 2-High Asthma. Cell Rep 32, 107872 (2020).
Lin, B., et al. Airway hillocks are injury-resistant reservoirs of unique plastic stem cells. Nature 629, 869-877 (2024).
La Manno, G., et al. RNA velocity of single cells. Nature 560, 494-498 (2018).
Bergen, V., Lange, M., Peidli, S., Wolf, F. A. & Theis, F. J. Generalizing RNA velocity to transient cell states through dynamical modeling. Nat Biotechnol 38, 1408-1414 (2020).
Poole, A., et al. Dissecting childhood asthma with nasal transcriptomics distinguishes subphenotypes of disease. J Allergy Clin Immunol 133, 670-678.e612 (2014).
Karp, T., et al. Nasal airway epithelium differentiates between asthma and healthy subjects and identifies clinically relevant asthma endotypes. European Respiratory Journal 60, 687 (2022).
Turner, J., et al. Goblet cells are derived from a FOXJ1-expressing progenitor in a human airway epithelium. Am J Respir Cell Mol Biol 44, 276-284 (2011).
Tyner, J. W., et al. Blocking airway mucous cell metaplasia by inhibiting EGFR antiapoptosis and IL-13 transdifferentiation signals. J Clin Invest 116, 309-321 (2006).
Ordonez, C. L., et al. Mild and moderate asthma is associated with airway goblet cell hyperplasia and abnormalities in mucin gene expression. Am J Respir Crit Care Med 163, 517-523 (2001).
Imkamp, K., et al. Gene network approach reveals co-expression patterns in nasal and bronchial epithelium. Sci Rep 9, 15835 (2019).
Raby, K. L., Michaeloudes, C., Tonkin, J., Chung, K. F. & Bhavsar, P. K. Mechanisms of airway epithelial injury and abnormal repair in asthma and COPD. Front Immunol 14, 1201658 (2023).
Dobin, A., et al. STAR: ultrafast universal RNA-seq aligner. Bioinformatics 29, 15-21 (2013).
Liao, Y., Smyth, G. K. & Shi, W. featureCounts: an efficient general purpose program for assigning sequence reads to genomic features. Bioinformatics 30, 923-930 (2014).
Risso, D., Schwartz, K., Sherlock, G. & Dudoit, S. GC-content normalization for RNA-Seq data. BMC Bioinformatics 12, 480 (2011).
Smedley, D., et al. BioMart—biological queries made easy. BMC Genomics 10, 22 (2009).
Robinson, M. D. & Oshlack, A. A scaling normalization method for differential expression analysis of RNA-seq data. Genome Biol 11, R25 (2010).
Law, C. W., Chen, Y., Shi, W. & Smyth, G. K. voom: Precision weights unlock linear model analysis tools for RNA-seq read counts. Genome Biol 15, R29 (2014).
Zhang, Y., Parmigiani, G. & Johnson, W. E.: batch effect adjustment for RNA-seq count data. NAR Genom Bioinform 2, lqaa078 (2020).
Johnson, W. E., Li, C. & Rabinovic, A. Adjusting batch effects in microarray expression data using empirical Bayes methods. Biostatistics 8, 118-127 (2007).
Hoffman, G. E. & Schadt, E. E. variancePartition: interpreting drivers of variation in complex gene expression studies. BMC Bioinformatics 17, 483 (2016).
Purcell, S., et al. PLINK: a tool set for whole-genome association and population-based linkage analyses. Am J Hum Genet 81, 559-575 (2007).
Howie, B., Fuchsberger, C., Stephens, M., Marchini, J. & Abecasis, G. R. Fast and accurate genotype imputation in genome-wide association studies through pre-phasing. Nat Genet 44, 955-959 (2012).
Pinheiro, J. & Bates, D. Mixed-Effects Models in S and S-PLUS. (2000).
Benjamini, Y. & Hochberg, Y. Controlling the False Discovery Rate: A Practical and Powerful Approach to Multiple Testing. Journal of the Royal Statistical Society: Series B (Methodological) 57, 289-300 (1995).
Mangiafico S. S. companion: Functions to Support Extension Education Program Evaluation. (2023).
Banchereau, R., et al. Personalized Immunomonitoring Uncovers Molecular Networks that Stratify Lupus Patients. Cell 165, 551-565 (2016).
Liberzon, A., et al. Molecular signatures database (MSigDB) 3.0. Bioinformatics 27, 1739-1740 (2011).
Wu, T., et al. clusterProfiler 4.0: A universal enrichment tool for interpreting omics data. Innovation (Camb) 2, 100141 (2021).
Korotkevich, G., et al. Fast gene set enrichment analysis. bioRxiv, 060012 (2021).
Satija, R., Farrell, J. A., Gennert, D., Schier, A. F. & Regev, A. Spatial reconstruction of single-cell gene expression data. Nat Biotechnol 33, 495-502 (2015).
Bakdash, J. Z. & Marusich, L. R. Repeated Measures Correlation. Front Psychol 8, 456 (2017).
Hubert, M., Rousseeuw, P. J. & Vanden Branden, K. ROBPCA: A New Approach to Robust Principal Component Analysis. Technometrics 47, 64-79 (2005).
Zhu, J., et al. Integrating large-scale functional genomic data to dissect the complexity of yeast regulatory networks. Nat Genet 40, 854-861 (2008).
Tran, L. M., et al. Inferring causal genomic alterations in breast cancer using gene expression data. BMC Syst Biol 5, 121 (2011).
Zhu, J., et al. Increasing the power to detect causal associations by combining genotypic and expression data in segregating populations. PLoS Comput. Biol 3, e69 (2007).
Shabalin, A. A. Matrix eQTL: ultra-fast eQTL analysis via large matrix operations. Bioinformatics 28, 1353-1358 (2012).
Garcia-Alonso, L., Holland, C. H., Ibrahim, M. M., Turei, D. & Saez-Rodriguez, J. Corrigendum: Benchmark and integration of resources for the estimation of human transcription factor activities. Genome Res 31, 745 (2021).
Giurgiu, M., et al. CORUM: the comprehensive resource of mammalian protein complexes-2019. Nucleic Acids Res 47, D559-D563 (2019).
Mullen, K. & van Stokkum, I. nnls: The Lawson-Hanson Algorithm for Non-Negative Least Squares (NNLS) (2023).
Newman, A. M., et al. Robust enumeration of cell subsets from tissue expression profiles. Nat Methods 12, 453-457 (2015).
Dong, M., et al. SCDC: bulk gene expression deconvolution by multiple single-cell RNA sequencing references. Brief Bioinform 22, 416-427 (2021).
Chu, T., Wang, Z., Pe'er, D. & Danko, C. G. Cell type and gene expression deconvolution with BayesPrism enables Bayesian integrative analysis across bulk and single-cell RNA sequencing in oncology. Nat Cancer 3, 505-517 (2022).
Edgar, R., Domrachev, M. & Lash, A. E. Gene Expression Omnibus: NCBI gene expression and hybridization array data repository. Nucleic Acids Res 30, 207-210 (2002).
Koller, M. robustlmm: An R Package for Robust Estimation of Linear Mixed-Effects Models. Journal of Statistical Software 75, 1-24 (2016).
Petpiroon, N., et al. Development of lung tissue models and their applications. Life Sci 334, 122208 (2023).
Zheng, G. X., et al. Massively parallel digital transcriptional profiling of single cells. Nat Commun 8, 14049 (2017).
Frankish, A., et al. GENCODE 2021. Nucleic Acids Res 49, D916-D923 (2021).
Wolock, S. L., Lopez, R. & Klein, A. M. Scrublet: Computational Identification of Cell Doublets in Single-Cell Transcriptomic Data. Cell Syst 8, 281-291.e289 (2019).
Hao, Y., et al. Dictionary learning for integrative, multimodal and scalable single-cell analysis. Nat Biotechnol 42, 293-304 (2024).
Korsunsky, I., et al. Fast, sensitive and accurate integration of single-cell data with Harmony. Nat Methods 16, 1289-1296 (2019).
Wolf, F. A., Angerer, P. & Theis, F. J. SCANPY: large-scale single-cell gene expression data analysis. Genome Biol 19, 15 (2018).

### EXAMPLES

The following examples are put forth so as to provide those of ordinary skill in the art with a complete disclosure and description of how to make and use the methods and compositions featured in the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example I

### Unraveling Asthma Pathophysiology: Integrative Network Analysis Implicates Mucous Ciliated Cell Dynamics in Response to Biologic Therapies—REGAIN Study

In this landmark study, the findings from REGAIN, the most extensive real-world asthma study of its kind, for which comprehensive longitudinal genomics, clinical, and digital health data were gathered are presented. This unprecedented dataset, encompassing 528 participants with asthma and 263 healthy participants, forms the foundation for an integrative network analysis, casting new light on asthma pathophysiology and treatment response. The central role of mucous ciliated and goblet cells in asthma severity and remission in response to treatment with biologic therapeutics are presented. Contrary to prevailing beliefs, this network-centric analysis reveals these cell types strongly associate with therapeutic response to biologics and predicts a protective role for mucous ciliated cells because mucous-ciliated cell frequency increased in those achieving remission in response to treatment but not in those who do not achieve remission. This study not only challenges current asthma paradigms, but also establishes a framework for understanding disease mechanisms and responses to treatment. To test the hypothesis derived from integrative analyses that mucous ciliated cells mediate a protective benefit, air-liquid interface cultured airway epithelial cell organoids were placed into a severe asthma state via exposure to IL-13. After removal of IL-13, mucous ciliated cells were directly observed to be instrumental in restoring normal organoids states, confirming the network-derived hypothesis presented herein. These findings offer a transformative perspective on asthma biology, potentially guiding more effective therapeutic strategies.

Asthma is a common chronic airway disease affecting both children and adults with significant heterogeneity in terms of clinical presentation including, but not limited to, symptom frequency and response to therapy. In asthma, the airway epithelium orchestrates immune responses to diverse environmental stimuli including microbes, allergens, and irritants. This response commonly involves epithelium-mediated initiation and maintenance of type 2 (T2) inflammation, featuring increased levels of eosinophils, T helper 2 (Th2) lymphocytes, allergen-specific immunoglobulin E (IgE) antibodies, mast cell activation, type 2 innate lymphoid cells (ILC2s) and interleukins (IL)-4, IL-5 and IL-13 (Hammad, H. & Lambrecht, B. N. The basic immunology of asthma. Cell 184, 1469-1485 (2021)). Less commonly, there may be low to absent levels of T2 inflammation and a neutrophilic or Th1/Th17 immune response may predominate (Sze, E., Bhalla, A. & Nair, P. Mechanisms and therapeutic strategies for non-T2 asthma. Allergy 75, 311-325 (2020)). Data suggest that repeated episodes of airway epithelial inflammation, injury and repair, followed by airway remodeling may impact the clinical course of asthma and response to therapies (Hough, K. P., et al. Airway Remodeling in Asthma. Front Med (Lausanne) 7, 191 (2020); Varricchi, G., et al. Biologics and airway remodeling in severe asthma. Allergy 77, 3538-3552 (2022)).

Monoclonal antibody therapies, also known as biologics, can achieve clinical remission of severe asthma as defined by minimal asthma signs and symptoms in approximately 20% of treated patients with asthma uncontrolled by inhaled therapies (Thomas, D., McDonald, V. M., Pavord, I.D. & Gibson, P. G. Asthma remission: what is it and how can it be achieved? Eur Respir J 60(2022)). There are currently no airway-based or blood-based biomarkers that consistently predict likelihood of clinical remission prior to treatment and molecular mechanisms underpinning clinical remission remain poorly understood. A challenge to investigating epithelial biology in asthma over time with treatment is the difficulty of performing repeated airway sampling by sputum induction or bronchoscopy longitudinally at scale required for adequately powered network analyses. Recent data support that nasal epithelial brushings recapitulate lower airway sampling in asthma and can be obtained serially and efficiently over time in order to overcome this barrier (Schatz, M., et al. Asthma Control Test: reliability, validity, and responsiveness in patients not previously followed by asthma specialists. J Allergy Clin Immunol 117, 549-556 (2006); Asthma, G. I. F. Global Strategy for Asthma Management and Prevention (2018)).

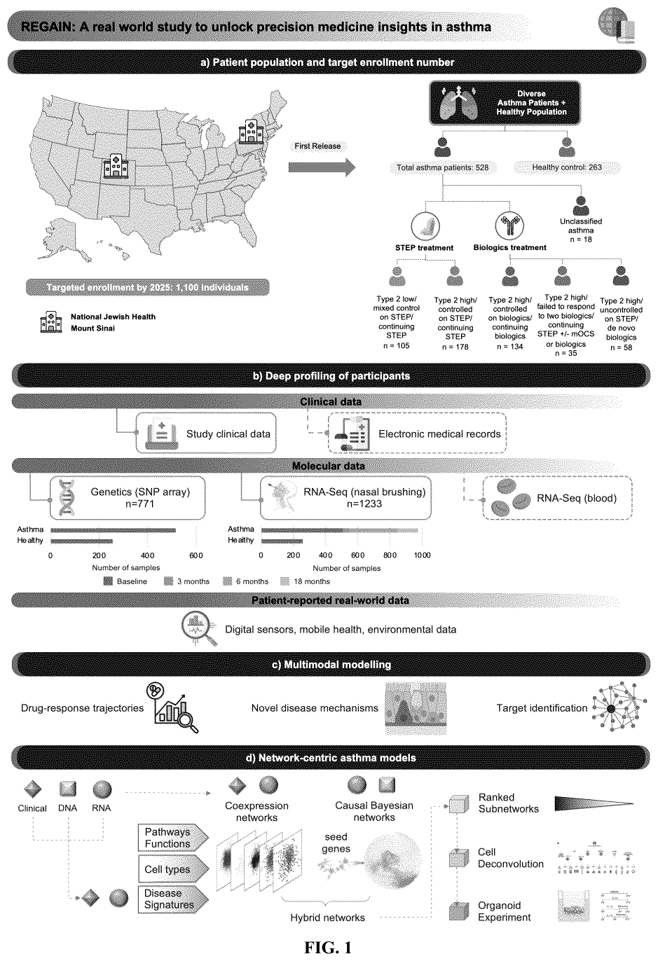

On this premise the REal-world and Genomic data-based Asthma Insights through Network analysis (REGAIN) study (FIG. 1A-FIG. 1D) was designed. REGAIN is a prospective 18-month longitudinal observational study of asthma in the course of routine clinical care at two North American healthcare systems intended to discover novel mechanisms of asthma pathogenesis, control, and remission. This overall initiative, a collaboration between academic health institutions and biotechnology and drug discovery partners, leverages a large multimodal data set and deep analytics to: 1) understand disease course and response to treatment; 2) uncover molecular mechanisms driving disease; and 3) inform novel target discovery. This report presents initial analyses of clinical trajectories of asthma participants using 1,233 nasal brushing RNA-Seq samples, and 771 genotype samples from 528 study participants with mild-to-severe asthma and 263 healthy controls (FIG. 1C). This unprecedented dataset forms the foundation for an integrative network analysis that leads to testable hypotheses relating to cellular aspects of asthma pathophysiology and treatment response (FIG. 1D).

### Results

### REGAIN is a Large Representative Cohort of Asthma Patients in Clinical Practice

Study participants were enrolled and contributed nasal brushing RNA-Seq for at least one study visit (baseline, 3, 6, or 18 months) and/or genotyping data. Nasal brushings were used as a surrogate for bronchial epithelial cells as has been previously reported (Schatz, M., et al. Asthma Control Test: reliability, validity, and responsiveness in patients not previously followed by asthma specialists. J Allergy Clin Immunol 117, 549-556 (2006); Asthma, G. I. F. Global Strategy for Asthma Management and Prevention (2018)). Participants with asthma were specifically enrolled according to five pre-specified groups, primarily classified by type of therapy during the study (Global Initiative of Asthma Step therapy (STEP) or biologics), inflammatory endotype (type 2 inflammation low/high), and asthma control (mixed/controlled/uncontrolled), reflecting a broad range of clinical and biological heterogeneity (Asthma, G. I. F. Global Strategy for Asthma Management and Prevention (2018)). These five groups were:
1) ‘Type 2 low/mixed control on STEP/continuing STEP’ (n=105, T2 low STEP);
2) ‘Type 2 high/controlled on STEP/continuing STEP’ (n=178, T2 STEP);
3) ‘Type 2 high/controlled on biologics/continuing biologics’ (n=134, T2 bio);
4) ‘Type 2 high/failed treatment to at least two prior biologics/continuing STEP+/−maintenance oral corticosteroids (mOCS) or biologics’ (n=35, T2 failed); and
5) ‘Type 2 high/uncontrolled on STEP/initiating treatment de novo with biologics’ (n=58, T2 de novo) (FIG. 1A and Table 1).

Additionally, asthma participants who did not meet the criteria for any pre-specified group were assigned as unclassified asthma (n=18). Those participants were included when analyzing all asthma participants and excluded from group-specific analyses. The demographics of the five analyzed asthma groups were generally comparable in terms of age, biologic sex, and self-reported race and ethnicity (Table 1). Healthy participants were enrolled for baseline cross-sectional comparison with asthma participants and were not followed longitudinally (Table 2).

TABLE 1
Baseline characteristics of REGAIN study participants with asthma by pre-specified group at enrollment.

| | | | | | | | Type 2 high/ | | | | |
|---|---|---|---|---|---|---|---|---|---|---|---|
| | | | | | | | failed to | | | | |
| | Type 2 | | | | Type 2 | | respond to | | | | |
| | low/ | | Type 2 | | high/ | | two | | Type 2 | | |
| | mixed | | high/ | | controlled | | biologics/ | | high/ | | |
| | control | | controlled | | on | | continuing | | uncontrolled | | |
| | on STEP/ | | on STEP/ | | biologics/ | | STEP +/− | | on STEP/ | | |
| | continuing | | continuing | | continuing | | mOCS or | | de novo | | P- |
| Variable¹ | STEP | | STEP | | biologics | | biologics | | biologics | | value² |
| Abbreviation | T2 low | | T2 STEP | | T2 bio | | T2 failed | | T2 de | | |
| | STEP | | | | | | | | novo | | |
| Type of therapy prior to | STEP | | STEP | | biologics | | biologics | | STEP | | |
| enrollment | | | | | | | | | | | |
| Inflammation type | type 2 | | type 2 | | type 2 | | type 2 | | type 2 | | |
| | low | | high | | high | | high | | high | | |
| Asthma control at | Mixed | | controlled | | controlled | | uncontrolled | | uncontrolled | | |
| baseline | control | | | | | | | | | | |
| Sample size, n | 105 | | 178 | | 134 | | 35 | | 58 | | |
| Study site, n (%) | | | | | | | | | | | 4.82E−7 |
| Mount Sinai | 45 | (43) | 127 | (71) | 73 | (54) | 25 | (71) | 46 | (79) | |
| NJH | 60 | (57) | 51 | (29) | 61 | (46) | 10 | (29) | 12 | (21) | |
| Age (year), mean ± s.d. | 54 ± 16 | | 56 ± 18 | | 58 ± 13 | | 54 ± 14 | | 55 ± 13 | | 0.29 |
| Biologic sex, n (%) | | | | | | | | | | | 0.054 |
| Male | 28 | (27) | 77 | (43) | 44 | (33) | 10 | (29) | 20 | (34) | |
| Female | 77 | (73) | 101 | (57) | 90 | (67) | 25 | (71) | 38 | (66) | |
| Ethnicity, n (%) | | | | | | | | | | | 0.62 |
| Hispanic | 21 | (20) | 37 | (21) | 33 | (25) | 12 | (34) | 17 | (29) | |
| Non-Hispanic | 82 | (78) | 139 | (78) | 99 | (74) | 23 | (66) | 41 | (71) | |
| Unknown/Not Reported | 2 | (2) | 2 | (1) | 2 | (1) | 0 | (0) | 0 | (0) | |
| Race, n (%) | | | | | | | | | | | 0.64 |
| White or European | 70 | (67) | 115 | (65) | 92 | (69) | 21 | (60) | 36 | (62) | |
| Black or African | 18 | (17) | 32 | (18) | 18 | (13) | 8 | (23) | 11 | (19) | |
| American | | | | | | | | | | | |
| American Indian or | 3 | (3) | 3 | (2) | 2 | (1) | 0 | (0) | 2 | (3) | |
| Alaskan Native | | | | | | | | | | | |
| Asian | 4 | (4) | 7 | (4) | 9 | (7) | 0 | (0) | 1 | (2) | |
| Native Hawaiian or | 0 | (0) | 0 | (0) | 0 | (0) | 2 | (6) | 0 | (0) | |
| Pacific Islander | | | | | | | | | | | |
| Mixed | 5 | (5) | 10 | (6) | 7 | (5) | 2 | (6) | 2 | (3) | |
| Unknown/Not Reported | 5 | (5) | 11 | (6) | 6 | (4) | 2 | (6) | 6 | (10) | |
| Education less than high | 1 | (1) | 4 | (2) | 4 | (3) | 6 | (17) | 6 | (10) | 2.74E−4 |
| school, n (%) | | | | | | | | | | | |
| BMI (kg/m²), mean ± s.d. | 28 ± 6 | | 29 ± 7 | | 29 ± 7 | | 33 ± 6 | | 31 ± 7 | | 5.04E−5 |
| BMI >30 kg/m²), n (%) | 34 | (32) | 62 | (35) | 47 | (35) | 22 | (63) | 31 | (53) | 0.002 |
| Age at diagnosis (year), | 29 ± 19 | | 29 ± 23 | | 32 ± 21 | | 17 ± 19 | | 27 ± 21 | | 0.005 |
| mean ± s.d. | | | | | | | | | | | |
| ACT score, mean ± s.d. | 19 ± 4 | | 22 ± 2 | | 22 ± 2 | | 12 ± 4 | | 13 ± 5 | | 7.14E−45 |
| GINA score, mean ± s.d. | 1.4 ± 1.3 | | 0.3 ± 0.7 | | 0.4 ± 0.7 | | 3.3 ± 0.9 | | 3 ± 1.3 | | 1.19E−52 |
| Exacerbation during | 21 | (20) | 56 | (31) | 48 | (36) | 26 | (74) | 43 | (74) | 2.8E−14 |
| past 12 months, n (%) | | | | | | | | | | | |
| ICU during past 12 | 0 | (0) | 3 | (2) | 2 | (1) | 1 | (3) | 2 | (3) | 0.33 |
| months, n (%) | | | | | | | | | | | |
| Comorbidity, n (%) | | | | | | | | | | | |
| Gastroesophageal reflux | 56 | (53) | 80 | (45) | 60 | (45) | 28 | (80) | 29 | (50) | 0.002 |
| disease (GERD) | | | | | | | | | | | |
| Vocal cord dysfunction | 42 | (40) | 34 | (19) | 31 | (23) | 16 | (46) | 22 | (38) | 7.58E−5 |
| (VCD) and/or laryngeal | | | | | | | | | | | |
| hypersensitivity | | | | | | | | | | | |
| Rhinosinusitis | 71 | (68) | 90 | (51) | 89 | (66) | 22 | (63) | 43 | (74) | 0.003 |
| Sleep apnea | 33 | (31) | 37 | (21) | 45 | (34) | 13 | (37) | 22 | (38) | 0.024 |
| Cardiovascular diseases³ | 41 | (39) | 64 | (36) | 51 | (38) | 17 | (49) | 25 | (43) | 0.64 |
| ICS or ICS/LABA dose, | | | | | | | | | | | 5E−4 |
| n (%)² | | | | | | | | | | | |
| Low | 30/91 | (33) | 61/160 | (38) | 26/117 | (22) | 4/33 | (12) | 5/58 | (9) | |
| Medium | 35/91 | (38) | 59/160 | (37) | 48/117 | (41) | 10/33 | (30) | 22/58 | (38) | |
| High | 26/91 | (29) | 40/160 | (25) | 43/117 | (37) | 19/33 | (58) | 31/58 | (53) | |
| Additional controller, n (%) | | | | | | | | | | | |
| LAMA | 21 | (20) | 21 | (12) | 35 | (26) | 21 | (60) | 28 | (48) | 2.61E−11 |
| LTM | 42 | (40) | 59 | (33) | 53 | (40) | 19 | (54) | 35 | (60) | 0.003 |
| Adjunctive therapy, n (%) | | | | | | | | | | | |
| Allergen | 5 | (5) | 9 | (5) | 3 | (2) | 0 | (0) | 0 | (0) | 0.24 |
| immunotherapy | | | | | | | | | | | |
| Antihistamine | 45 | (43) | 65 | (37) | 47 | (35) | 20 | (57) | 23 | (40) | 0.15 |
| Macrolide | 2 | (2) | 1 | (1) | 2 | (1) | 5 | (14) | 3 | (5) | 6.21E−4 |
| Nasal steroids | 46 | (44) | 77 | (43) | 63 | (47) | 14 | (40) | 28 | (48) | 0.90 |
| Maintenance OCS, n (%)⁵ | 1 | (1) | 4 | (2) | 5 | (4) | 13 | (37) | 9 | (16) | 4.03E−11 |
| Current biological | | | | | | | | | | | |
| therapies, n (%) | | | | | | | | | | | |
| Omalizumab | 0 | (0) | 0 | (0) | 19 | (14) | 0 | (0) | 0 | (0) | 3.74E−10 |
| Dupilumab | 0 | (0) | 0 | (0) | 59 | (44) | 13 | (37) | 0 | (0) | 3.82E−38 |
| Mepolizumab | 0 | (0) | 0 | (0) | 21 | (16) | 1 | (3) | 0 | (0) | 5.42E−11 |
| Reslizumab | 0 | (0) | 0 | (0) | 1 | (1) | 2 | (6) | 0 | (0) | 0.012 |
| Benralizumab | 0 | (0) | 0 | (0) | 31 | (23) | 7 | (20) | 0 | (0) | 3.73E−18 |
| Tezepelumab | 0 | (0) | 0 | (0) | 3 | (2) | 3 | (9) | 0 | (0) | 0.001 |
| Past biological | | | | | | | | | | | |
| therapies, n (%)⁴ | | | | | | | | | | | |
| Omalizumab | 3 | (3) | 1 | (1) | 14 | (10) | 23 | (66) | 5 | (9) | 8.11E−22 |
| Dupilumab | 0 | (0) | 3 | (2) | 6 | (4) | 12 | (34) | 3 | (5) | 7.39E−10 |
| Mepolizumab | 0 | (0) | 2 | (1) | 12 | (9) | 23 | (66) | 2 | (3) | 8.43E−24 |
| Reslizumab | 0 | (0) | 0 | (0) | 3 | (2) | 8 | (23) | 0 | (0) | 5.66E−9 |
| Benralizumab | 0 | (0) | 1 | (1) | 10 | (7) | 15 | (43) | 3 | (5) | 1.41E−14 |

¹BMI: body mass index; ICU: intensive care unit; GERD: gastroesophageal reflux disease; VCD: vocal cord dysfunction; ICS: inhaled corticosteroid; LABA: long-acting β2-agonist; LAMA: long-acting muscarinic antagonist; LTM: leukotriene modifier; OCS: oral corticosteroids; ACT: Asthma Control Test, GINA Score: Global Initiative for Asthma symptom control, based on GINA 2018;
²P-values were derived from two-sided Fisher's exact test for categorical variables and Kruskal-Wallis test for numeric variables;
³Including hypertension, coronary artery disease, and congestive heart failure;
⁴Patients might have been treated with multiple biologics in the past, so the percentages might not sum to 100%;
⁵A total of 5 patients on maintenance OCS were inadvertently enrolled in the STEP therapy groups.

TABLE 2
Baseline characteristics of REGAIN study participants analyzed
in this report by study site and case-control status.

| | |
|---|---|
| Mount Sinai (n = 491) | NJH (n = 300) |

| | Asthma | | Healthy | | P- | Asthma | | Healthy | | P- |
|---|---|---|---|---|---|---|---|---|---|---|
| Variable | patient | | volunteer | | value* | patient | | volunteer | | value* |
| Sample size, n | 317 | | 174 | | | 211 | | 89 | | |
| Age (year), mean ± s.d. | 53 ± 16 | | 48 ± 17 | | 0.001 | 59 ± 15 | | 55 ± 11 | | 0.007 |
| Biologic sex, n (%) | | | | | 0.85 | | | | | 0.40 |
| Male | 118 | (37.2) | 67 | (38.5) | | 64 | (30.3) | 22 | (24.7) | |
| Female | 199 | (62.8) | 107 | (61.5) | | 147 | (69.7) | 67 | (75.3) | |
| Ethnicity, n (%) | | | | | 7.26E−4 | | | | | 0.26 |
| Hispanic | 103 | (32.5) | 32 | (18.4) | | 20 | (9.5) | 9 | (10.1) | |
| Non-Hispanic | 214 | (67.5) | 142 | (81.6) | | 184 | (87.2) | 80 | (89.9) | |
| Unknown/Not | 0 | (0) | 0 | (0) | | 7 | (3.3) | 0 | (0) | |
| Reported | | | | | | | | | | |
| Race, n (%) | | | | | 0.029 | | | | | 0.16 |
| White or | 170 | (53.6) | 104 | (59.8) | | 179 | (84.8) | 78 | (87.6) | |
| European | | | | | | | | | | |
| Black or African | 79 | (24.9) | 44 | (25.3) | | 9 | (4.3) | 1 | (1.1) | |
| American | | | | | | | | | | |
| American Indian | 5 | (1.6) | 3 | (1.7) | | 5 | (2.4) | 1 | (1.1) | |
| or Alaskan Native | | | | | | | | | | |
| Asian | 18 | (5.7) | 13 | (7.5) | | 4 | (1.9) | 5 | (5.6) | |
| Native Hawaiian | 2 | (0.6) | 1 | (0.6) | | 0 | (0) | 0 | (0) | |
| or Pacific Islander | | | | | | | | | | |
| Mixed | 17 | (5.4) | 7 | (4) | | 10 | (4.7) | 1 | (1.1) | |
| Unknown/Not | 26 | (8.2) | 2 | (1.1) | | 4 | (1.9) | 3 | (3.4) | |
| Reported | | | | | | | | | | |
| Education less | 21 | (6.6) | 1 | (0.6) | 0.001 | 0 | (0) | 0 | (0) | NA |
| than high school, | | | | | | | | | | |
| n (%) | | | | | | | | | | |
| BMI (kg/m²), | 29 ± 7 | | 27 ± 6 | | 7.32E−6 | 30 ± 8 | | 26 ± 5 | | 2.36E−6 |
| mean ± s.d. | | | | | | | | | | |
| Obesity (BMI >30 | 124 | (39.1) | 39 | (22.4) | 1.98E−4 | 80 | (37.9) | 16 | (18) | 6.71E−4 |
| kg/m²), n (%) | | | | | | | | | | |

| | Combined (n = 791) | | | | |
|---|---|---|---|---|---|
| | Asthma | | Healthy | | P- |
| Variable | patient | | volunteer | | value* |
| Sample size, n | 528 | | 263 | | |
| Age (year), mean ± s.d. | 55 ± 16 | | 51 ± 16 | | 3.01E−5 |
| Biologic sex, n (%) | | | | | 0.87 |
| Male | 182 | (34.5) | 89 | (33.8) | |
| Female | 346 | (65.5) | 174 | (66.2) | |
| Ethnicity, n (%) | | | | | 0.004 |
| Hispanic | 123 | (23.3) | 41 | (15.6) | |
| Non-Hispanic | 398 | (75.4) | 222 | (84.4) | |
| Unknown/Not | 7 | (1.3) | 0 | (0) | |
| Reported | | | | | |
| Race, n (%) | | | | | 0.093 |
| White or | 349 | (66.1) | 182 | (69.2) | |
| European | | | | | |
| Black or African | 88 | (16.7) | 45 | (17.1) | |
| American | | | | | |
| American Indian | 10 | (1.9) | 4 | (1.5) | |
| or Alaskan Native | | | | | |
| Asian | 22 | (4.2) | 18 | (6.8) | |
| Native Hawaiian | 2 | (0.4) | 1 | (0.4) | |
| or Pacific Islander | | | | | |
| Mixed | 27 | (5.1) | 8 | (3) | |
| Unknown/Not | 30 | (5.7) | 5 | (1.9) | |
| Reported | | | | | |
| Education less | 21 | (4) | 1 | (0.4) | 0.002 |
| than high school, | | | | | |
| n (%) | | | | | |
| BMI (kg/m²), | 29 ± 7 | | 26 ± 6 | | 2.83E−10 |
| mean ± s.d. | | | | | |
| Obesity (BMI >30 | 204 | (38.6) | 55 | (20.9) | 3.73E−7 |
| kg/m²), n (%) | | | | | |

*P-values were derived from two-sided Fisher's exact test for categorical variables and from Kruskal-Wallis test for numeric variables.

### Longitudinal Disease Trajectories of Different Asthma Groups

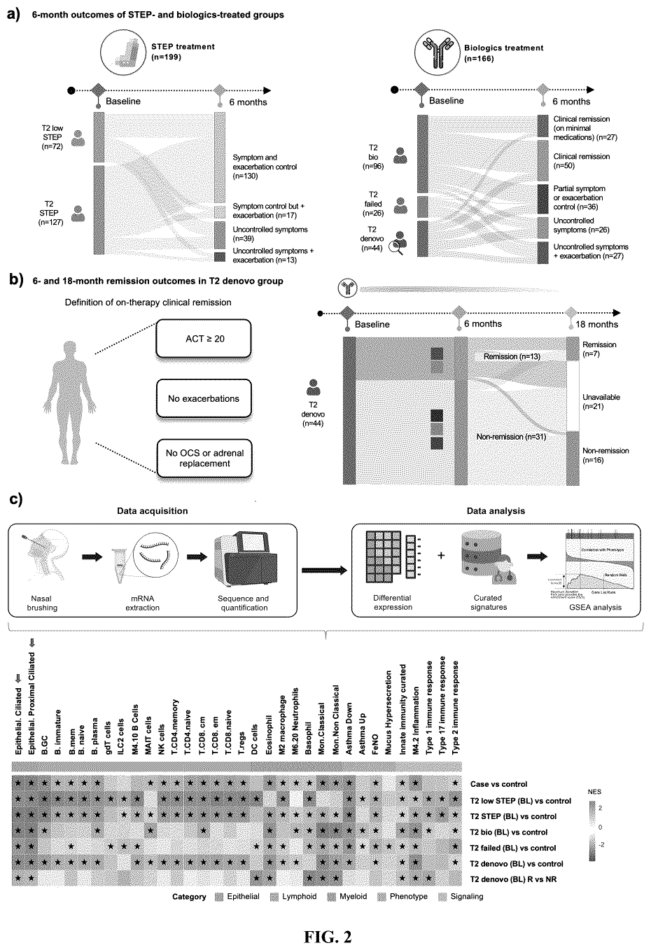

Asthma control was examined over time in the two STEP-treated and three biologics-treated groups defined at baseline with reference to symptom measures and exacerbation control at 6 months (FIG. 2A and Table 3). As expected, most participants in the type 2 controlled groups, both ‘T2 STEP’ and ‘T2 bio,’ retained a higher level of control and demonstrated minimal change in clinical course at 6 months after treatment. For a subset of participants consisting of 34 ‘T2 STEP’ participants (26.8%) and 34 ‘T2 bio’ participants (35.4%), disease status worsened after 6 months. The ‘T2 low STEP’ group, by design, included participants with different levels of asthma control at baseline and, as expected, a more heterogeneous clinical course. In total, 37 (51.4%) of these participants achieved disease control and 35 (48.6%) were uncontrolled at 6 months. In contrast to these three groups, and as expected, a substantial proportion of ‘T2 failed’ participants (n=24, 92.3%) exhibited persistent lack of control. The ‘T2 de novo’ group was unique in that all members of this group had low control at baseline despite high intensity inhaled therapy leading to the initiation of biologic treatment. Because participants in this group were naive to biologics or off prior biologic treatment for at least 4 months at baseline, this was the ideal group to investigate the impact of these treatments.

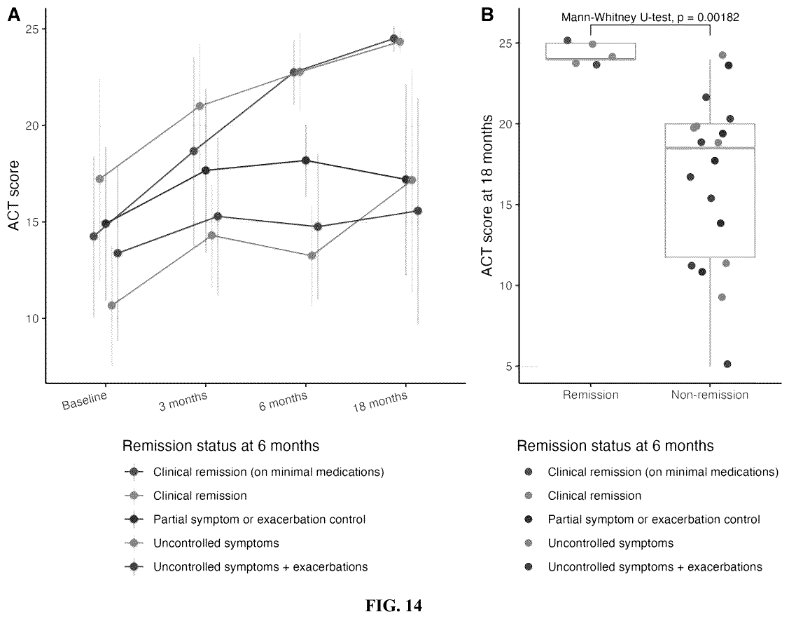

In this group, different levels of disease control were observed after treatment, ranging from uncontrolled symptoms with exacerbations (n=12, 18.2%), to uncontrolled symptoms without exacerbations (n=8, 27.3%), to partial remission (n=11, 25%, defined as moderate symptom or exacerbation control), to clinical remission (n=9, 20.5%, defined as a high degree of symptom control with biologics added to inhaled therapy), to clinical remission on minimal at medications (n=4, 9%, defined as a high degree of symptom control in presence of significantly reduced inhaled therapy) (FIG. 2). In subsequent analyses, the two clinical remission levels assessed after 6 months of treatment are referred to as ‘remission’ whilst the other three levels with worse outcomes are referred to as ‘non-remission’. This 6-month based definition is supported by the observation that, in the ‘T2 de novo’ group, most of the patients maintain the same remission status after 18 months (82.6%, P=0.02, FIG. 2B). Patients with remission at 6 months showed indeed higher levels of ACT scores at 18 months (remission vs. non-remission at 18 months: ACT mean 24.4 vs. 16.6, Mann-Whitney U-test P=0.00182, FIG. 14).

TABLE 3
Definition of remission or control status at six months for participants treated with or without biologics.
A. Treated with biologics: ‘T2 bio’, ‘T2 failed’, and ‘T2 de novo’.

| | | # | | # | # | # |
|---|---|---|---|---|---|---|
| Remission | ACT and | Exacerbations | | participants | participants | participants |
| status at 6 | GINA at | over last | Current | in | in | in |
| months | 6 months | 6 months | medication/treatment | ‘T2 bio’ | ‘T2 failed’ | ‘T2 de novo’ |
| Clinical | ACT ≥ 20 | 0 | On no maintenance | 22 | 1 | 4 |
| Remission | | | inhaled therapy, low dose | | | |
| (on | | | inhaled ICS or | | | |
| minimal | | | ICS/LABA,¹ or as needed | | | |
| medications) | | | ICS/Formoterol | | | |
| | | | AND | | | |
| | | | No chronic maintenance | | | |
| | | | OCS or adrenal | | | |
| | | | replacement² | | | |
| Clinical | ACT ≥ 20 | 0 | On medium to high dose | 40 | 1 | 9 |
| Remission | | | maintenance ICS or | | | |
| | | | ICS/LABA¹ | | | |
| | | | AND | | | |
| | | | No chronic maintenance | | | |
| | | | OCS or adrenal | | | |
| | | | replacement² | | | |
| Partial | ACT 16- | 0 | On medium to high dose | 23 | 2 | 11 |
| symptom or | 19 and | | maintenance ICS or | | | |
| exacerbation | GINA ≤ 2 | | ICS/LABA¹ | | | |
| control | ACT ≥ 20 | 1 | AND | | | |
| | | | (No chronic maintenance | | | |
| | | | OCS or adrenal | | | |
| | | | replacement² | | | |
| | | | OR | | | |
| | | | On chronic maintenance | | | |
| | | | OCS or adrenal | | | |
| | | | replacement, but | | | |
| | | | reduction of maintenance | | | |
| | | | dose from baseline ≥50%) | | | |
| Uncontrolled | ACT 16- | 0 | On medium to high dose | 6 | 8 | 12 |
| symptoms | 19 and | | maintenance ICS or | | | |
| | GINA ≥ 3 | | ICS/LABA¹ | | | |
| | OR | | AND | | | |
| | ACT < 16 | | (No chronic maintenance | | | |
| | | | OCS or adrenal | | | |
| | | | replacement² | | | |
| | | | OR | | | |
| | | | On chronic maintenance | | | |
| | | | OCS or adrenal | | | |
| | | | replacement, but | | | |
| | | | reduction of maintenance | | | |
| | | | dose from baseline <50%) | | | |
| Uncontrolled | ACT 16- | ≥1 | On medium to high dose | 5 | 14 | 8 |
| symptoms + | 19 and | | maintenance ICS or | | | |
| exacerbation | GINA ≥ 3 | | ICS/LABA¹ | | | |
| | OR | | AND | | | |
| | ACT < 16 | | | | | |
| | ACT ≥ 20 | >1 | (No chronic maintenance | | | |
| | | | OCS or adrenal | | | |
| | | | replacement² | | | |
| | | | OR | | | |
| | | | On chronic maintenance | | | |
| | | | OCS or adrenal | | | |
| | | | replacement, but | | | |
| | | | reduction of maintenance | | | |
| | | | dose from baseline <50%) | | | |

B. Treated without biologics: ‘T2 STEP’ and ‘T2 low STEP’

| | | | # | # |
|---|---|---|---|---|
| | | Exacerbations | participants | participants |
| | ACT at | over last | in | in ‘T2 |
| Control status at 6 months | 6 months | 6 months | ‘T2 STEP’ | low STEP’ |
| Symptom and exacerbation control | ≥20 | No | 93 | 37 |
| Symptom control + exacerbation | ≥20 | Yes | 10 | 7 |
| Uncontrolled symptoms | <20 | No | 18 | 21 |
| Uncontrolled symptoms + | <20 | Yes | 6 | 7 |
| exacerbation | | | | |

¹ICS dose equivalencies modified from GINA 2018.
²Prednisone ≤5 mg or equivalent (prednisone 5 mg = hydrocortisone 20 mg = methylprednisolone 4 mg = dexamethasone 0.8 mg).

### Type 2-High and Type 2-Low Patient Groups Showed Distinct Transcriptional Phenotypes at Baseline

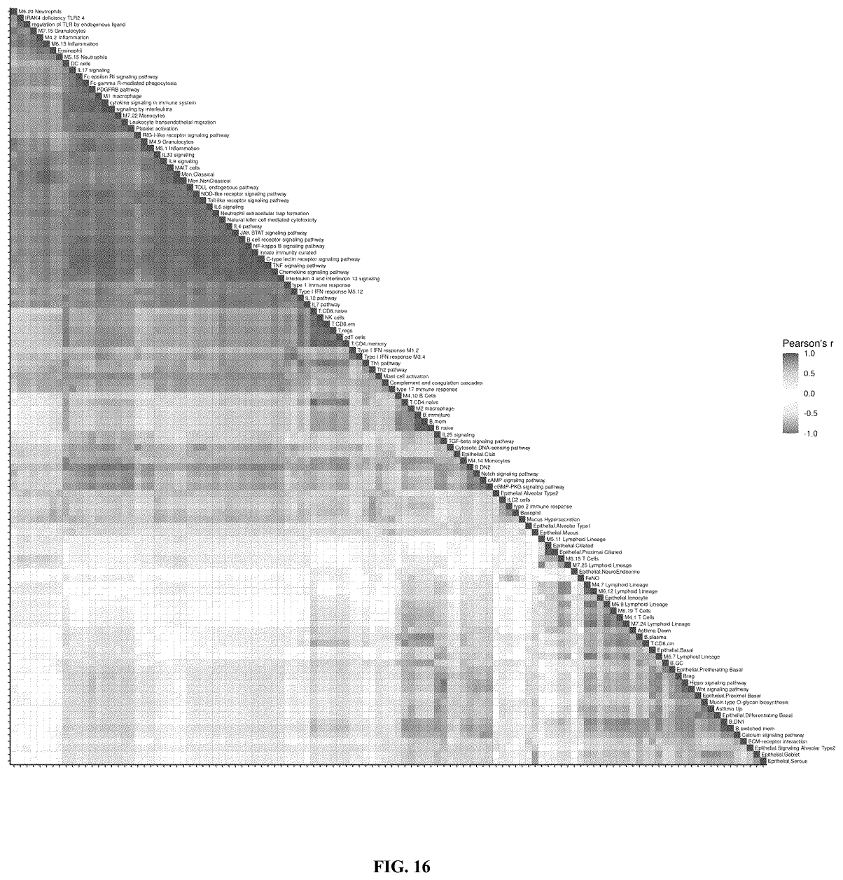

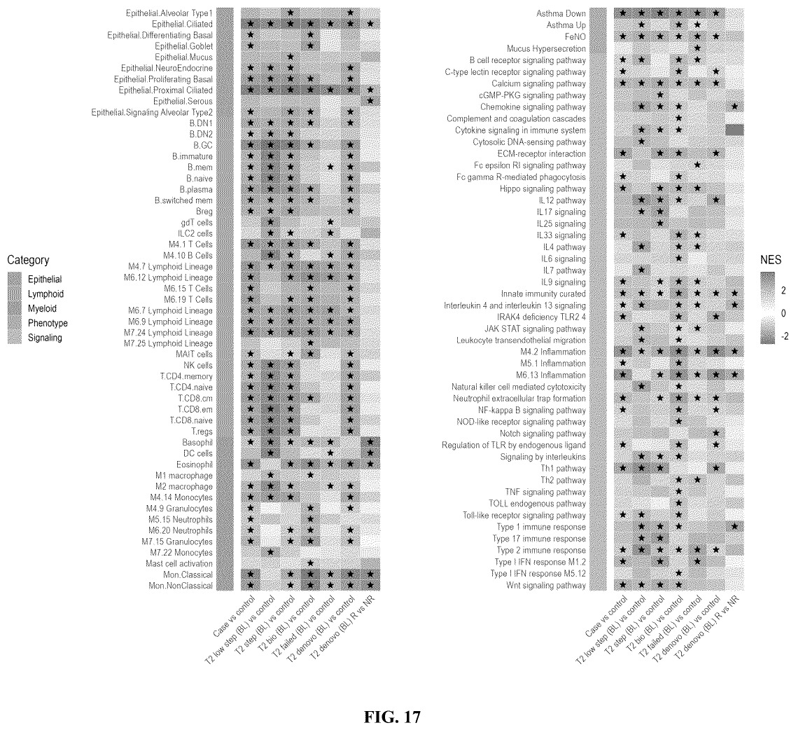

First, the transcriptomic signatures of asthma patients were characterized in aggregate and by subgroup by identifying their disease-specific gene expression changes at baseline compared to healthy participants. To increase statistical power for detecting robust signals and to minimize the impact of gene-level noise, a pathway-level analysis was performed using a gene set enrichment analysis (GSEA) method using 116 curated asthma signatures (FIG. 16 and FIG. 17).

Consistent with prior knowledge, gene sets associated with eosinophil biology, production of exhaled nitric oxide (FeNO), and type 2 immune response were upregulated compared to healthy controls across type 2 participants (‘T2 STEP’, ‘T2 de novo’, ‘T2 bio’, and ‘T2 failed’), but not in the ‘type 2-low’ group. ‘Type 2’ participants also showed significant upregulation of gene sets associated with basophils, neutrophils, classical/non-classical monocytes, and innate immune signaling, consistent with a type 2 predominant inflammatory milieu. Moreover, upregulation of the gene set associated with mucosal-associated invariant T (MAIT) cells, a cell type associated with airflow limitation in asthmatic patients, was observed. Along with previously known and expected specificities, the ‘type 2-high’ and ‘type 2-low’ also share many features when compared to healthy controls. These included a lower expression of genes associated with epithelial ciliated cell function, T and B cell inflammation, and higher expression of genes involved in leukocyte trafficking and innate immune responses. In the ‘T2 failed’ group, a unique expression pattern was identified with upregulation of gene sets associated with B cells, M2 macrophages, ILC2, gamma-delta T cells (gdT), dendritic cells (DCs), and mucous hypersecretion. These findings demonstrate that the molecular and cellular mechanisms driving this asthma subtype are distinct. These new mechanisms may help guide development of targeted therapies for this specific asthma subpopulation to maximize efficacy.

### Longitudinal Gene Expression Analysis Implicated Ciliated Cells in Asthma Remission

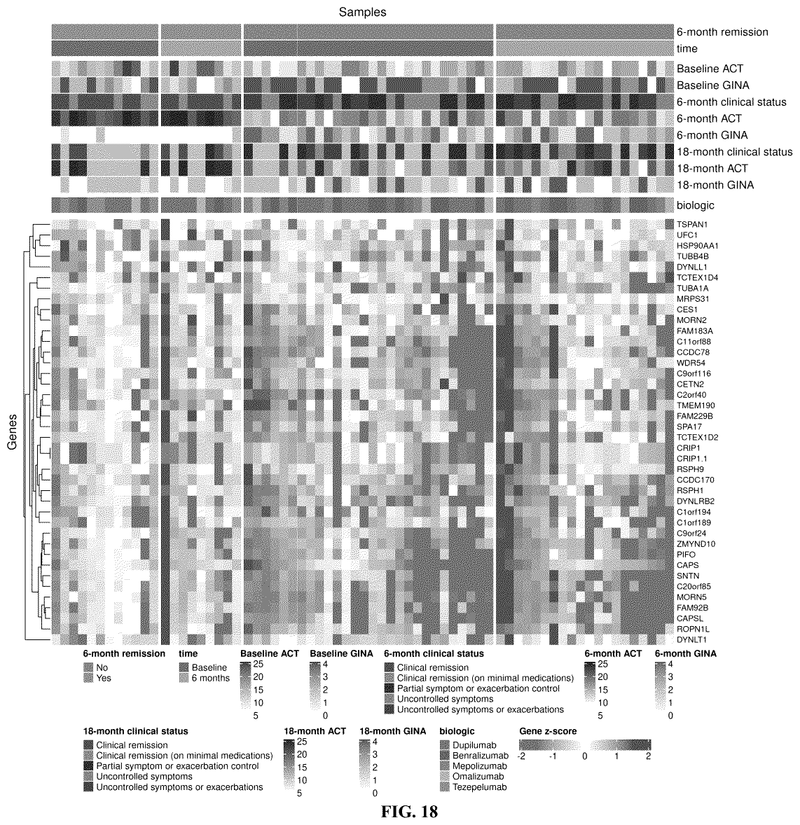

Next, gene expression patterns associated with different clinical responses in the ‘T2 de novo’ participants were focused upon, with all biologic therapies pooled as a group. GSEA revealed higher baseline expression of gene sets associated with ciliated cells (‘epithelial ciliated‘and’epithelial proximal ciliated’) in patients achieving remission compared to those who did not, suggesting this cell type being associated with clinical response to biologics treatment (FIG. 2C and FIG. 17). Looking at the longitudinal transcriptomic profiles of this asthma group in response to biologics treatment, non-remission participants experienced a greater fold increase in expression of gene sets associated with ciliated cells at 6 months after treatment (P<0.05, FIG. 18). Despite such increase in treatment response, non-remission participants on pooled biologics treatments exhibited persistently lower expression of ciliary genes at 6 months (P<0.001) (FIG. 18).

Taken together, these results demonstrate that nasal brushing RNA data can capture substantial portions of airway biology and provides an opportunity to discover molecular mechanisms underlying treatment-induced remission.

### Organization of High-Dimensional Molecular Data Relating to Network Components

### The Network Approach

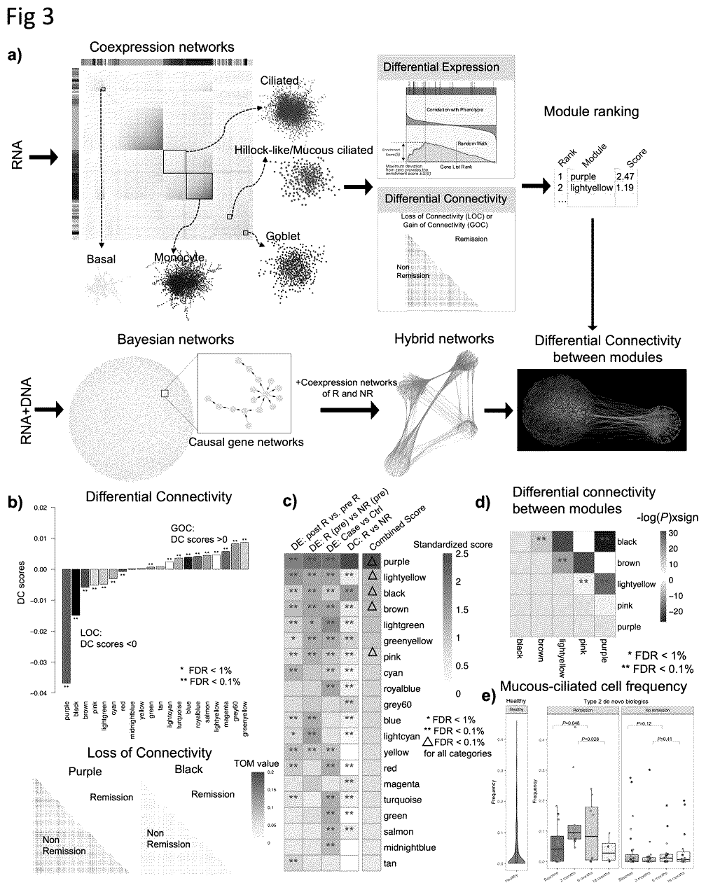

As a framework to leverage holistically the rich correlation structures represented in the data generated in REGAIN, coexpression and Bayesian networks were constructed that considered all relationships among the gene expression traits, including statistically inferred causal relationships (FIG. 3A). With these networks the complexity of response to biologics in severe asthma study participants (defined as those not well controlled by STEP) was elucidated by identifying those components of the network that were most different between the remission and non-remission groups. These differences manifested not only as nodes in the network that are differentially expressed, but also as changes in the network structure itself that are driven by differentially coregulated groups of genes reflecting core molecular and cellular functions. To detect such differences, we identified coexpression modules (subnetworks of genes that are highly interconnected with each other) that were not only enriched for genes that were differentially expressed between response groups, but that were also differentially connected both within and between these subnetworks. The differentially coregulated substructures in this network model therefore reflected regulatory mechanisms underlying remission associated with biologic treatment in REGAIN.

### Establishing Baseline Network Characteristics

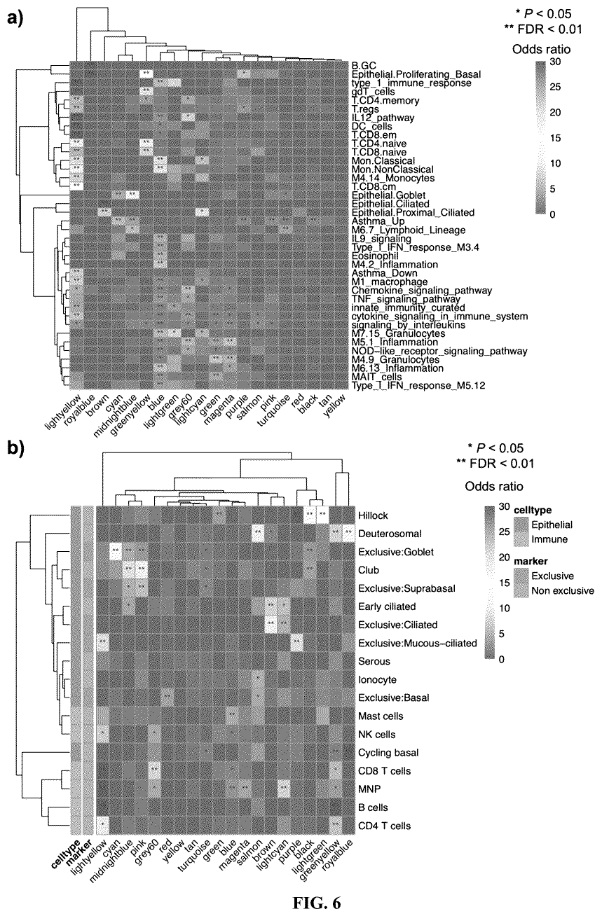

A coexpression network was built on samples collected at the baseline visit on all asthma and healthy volunteer participants to establish baseline characteristics of the molecular networks in airway epithelium and detect baseline modules. Overall, 20 modules were identified representing clusters of genes that are more strongly co-regulated with each other than they are with genes outside the module. To understand the biological functions of these different gene subnetworks and their association with asthma, we annotated the modules in several ways. First, pathway enrichment analysis was performed to understand the biological processes and molecular functions represented in each module (FIG. 6A), which was then further annotated with cell type marker genes and cellular processes to understand the composition of cell types represented in the network and their respective functions (FIG. 6B). Many modules were enriched for asthma-related pathways and cell type annotations, indicating good power to detect expected biological signals in the cohort. (FIG. 6A, FIG. 6B and Table 4). Specifically, modules were identified that were enriched in immune and inflammation processes such as cytokine signaling in the ‘light-yellow’ modules, eosinophil activity in the blue module (FIG. 6A), and a variety of epithelial functions known to be involved in asthma such as Hillock-like cells in the ‘purple’ module, goblet cells in ‘black’, ‘cyan’, and ‘pink’ modules, basal cells in ‘green-yellow’, ciliated cells in the ‘brown’ module, and mucous-ciliated cells in ‘purple’ and ‘light-yellow’ modules (FIG. 6B).

TABLE 4

| Module | |
|---|---|
| (indicated | |
| by color) | Cell type and pathway annotation |
| blue | related to eosinophils, mast cells, monocytes, chemokine |
| | signaling, cytokine signaling, interleukin signaling, and innate |
| | immune responses |
| light- | associated with B cells, T cells, mucous-ciliated cells, |
| yellow | cytokine signaling, and innate immune responses |
| green | associated with mucosal associated invariant T (MAIT) cells, |
| | Hillock-like cells, and cytokine signaling |
| brown | distinctly associated with ciliated cells |
| green- | associated with cycling basal cells, B cells, T cells, and |
| yellow | gamma delta T (gdT) cells |
| royal blue | associated with cycling basal cells, deuterostomal cells, and |
| | germinal center (GC) B cells |
| purple | associated with both Hillock-like cells and mucous-ciliated |
| black | cells associated with goblet cells, Hillock-like cells, and club |
| pink | cells linked to club cells, goblet cells, and superbasal cells |

### Examining Network Changes Related to Asthma Pathogenesis and Treatment Responses

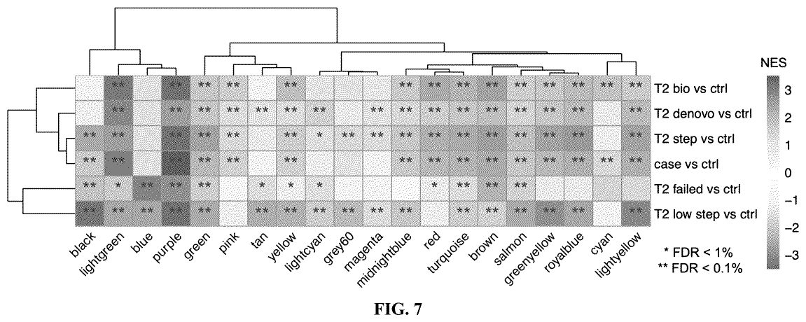

Each module's relevance in asthma by relating differential expression and network connectivity to asthma pathology and treatment response in REGAIN was then ranked (FIG. 3A). The first component of this ranking system was differential module enrichment for disease signatures derived from comparing all REGAIN asthma cases and controls (FIG. 7). In fact, all modules were found to be enriched for genes associated with asthma cases vs. controls and/or within different asthma subgroups compared to controls, indicating many pathophysiological mechanisms are captured by the regulatory processes in cells collected in the nasal brushings, which were reflected in our baseline network (FIG. 7). Among these, ‘purple’, ‘black’, and ‘light-green’ were upregulated in asthma case vs. control. In contrast, modules such as ‘brown’, ‘light-yellow’, and ‘royal blue’ were downregulated in asthma case vs. control.

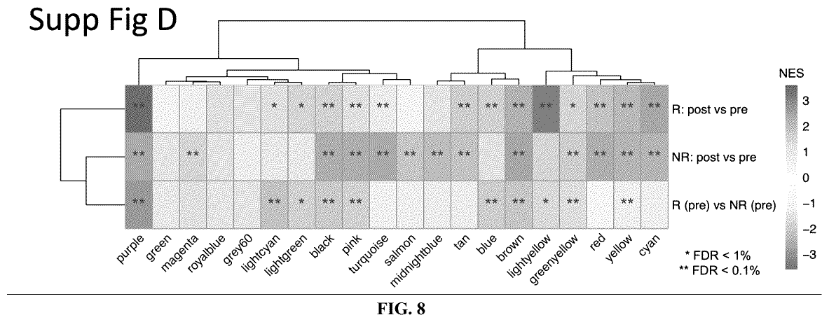

To derive additional ranking components, we next examined expression and network changes over time to elucidate therapeutic response (FIG. 3A). Patients who remained uncontrolled despite multiple lines of biologic treatments represented a critical subset with limited therapeutic options. Identifying the differential molecular programs between those who respond to such treatments and those who do not may serve to address an unmet medical need by suggesting alternative therapeutic mechanisms and may also aid in the development of biomarkers to better predict and enhance the efficacy of biologic treatments in patients resistant to conventional STEP approaches. To elucidate the differences in therapeutic response, the second and third ranking components were defined as modules enriched for differential expression (DE) signals between the ‘T2 de novo’ R and NR groups at baseline, and between the baseline and post-biologic treatments within the R group. While many of the modules in the baseline network were enriched for at least one of these (DE) signals (FIG. 8), ‘purple’ was the most downregulated and ‘light-yellow’ was the most upregulated module during remission. One noteworthy pattern specific to the ‘purple’, ‘black’ and ‘pink’ modules is that each of these was upregulated post-treatment with biologics (compared to the baseline state) in the NR group but downregulated in the R group and showed higher expression at baseline in R vs NR.

For the fourth and final ranking criterion, the molecular programs associated with response and non-response in those patients placed on new biologics therapies (‘T2 de novo’) were characterized by constructing additional coexpression networks on R and NR separately. For these two groups, all time points (baseline, 3-month, 6-month, 18-month) were pooled and a repeated measures correlation statistic was used to construct the coexpression networks. The ‘purple’ and ‘black’ modules defined at baseline showed the strongest within-module differential connectivity (DC) between remission and non-remission, highlighting a potential role for them in mediating response to biologics to achieve remission. Interestingly, within-module connectivity was reduced in both modules during remission, while most other modules showed a gain of connectivity, demonstrating that the reduction in connectivity in ‘black’ and ‘purple’ is unlikely to represent an artefact of the R and NR population size (FIG. 3B). Furthermore, all modules were rank-ordered by integrating all four ranking criteria (FIG. 3C), identifying ‘purple’ as the top two most significant module relevant to asthma pathology and response to new biologics (FIG. 3C). In addition, ‘light-yellow’, ‘black’, ‘brown’, and ‘pink’ also met stringent enrichment cutoffs (FDR <0.001) across all DE and DC criteria (FIG. 3C).

The Hybrid Networks as a Way to Characterize Interactions Between Modules and their Association to Response to Biologics

With the ‘purple’, ‘black’, ‘pink’, ‘light-yellow’, and ‘brown’ modules largely representing Hillock-like, goblet, club/goblet, and mucous-ciliated/B/T, and ciliated cell types (FIG. 6B), in addition to a mix of immune cell types in ‘light-yellow’, and given their enrichment for the asthma and response signatures, these results indicated that interactions among these modules (cell types) may be critical for effective response to treatment with biologics. Given the dominance of the ‘purple’ module and its similarity to the ‘black’ module in terms of differential expression and differential connectivity patterns (FIG. 7, FIG. 8 and FIG. 3B), the hypothesis that basal-to-Hillock-like and basal-to-goblet/ciliated cell type transitions are two incompatible differentiation processes that cannot co-exist was examined. Towards this end, whether the connectivity in the R and NR networks between the ‘purple’ module and the other top modules was associated with remission and the correspondence of these differential connectivities with cell states was assessed.

A more integrative network comprised of a down-sampled set of edges from the coexpression networks as well as edges from a Bayesian network (BN) constructed on the full baseline dataset was constructed. The BN provided a source of statistically inferred causal relationships between the gene expression traits, and edges sampled from the matrix of all pairwise gene connectivity scores was used to construct the coexpression networks, representing a broader range of topological properties, including centrality characteristics in addition to topological overlap. In contrast to WGCNA networks, which only prioritize topological overlap and associative relationships, the integrative networks captured centrality and some inferred causal relationships as well those that are important for assessing inter-module connectivity.

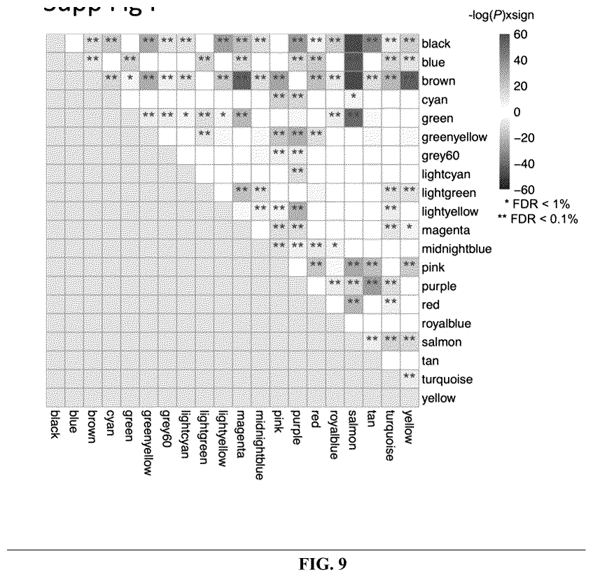

Whether the proportion of edges between the ‘purple’ module and each of the ‘black’, ‘pink’, ‘brown’, and ‘light-yellow’ modules were different between the R and NR integrative networks using a two-sample Z test of proportions was tested. It was determined that that ‘black’, and ‘light-yellow’ were strongly differentially connected to the ‘purple’ module (FDR <0.1%), whereas ‘brown’ and ‘pink’ were not found to be differentially connected to ‘purple’. Specifically, we observed a significant loss of connectivity between ‘black’ and ‘purple’, whereas we found a gain of connectivity between ‘light-yellow’ and ‘purple’, as well as between ‘light-yellow’ and ‘black’ (FIG. 3D and FIG. 9). These results indicated potential transitions between cell types represented by these modules in response to biologic treatment.

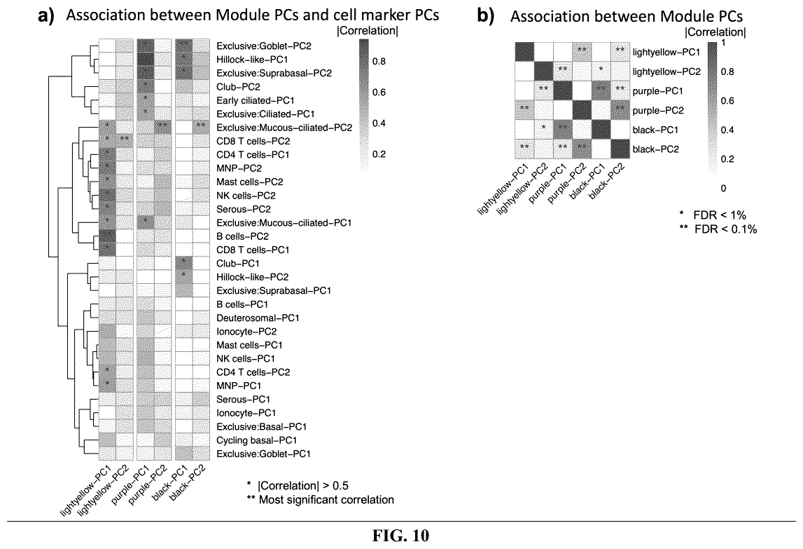

To understand in detail which cell types were mediating the observed network changes between these three modules, the previously described enrichment analysis was extended with additional Principal Component analysis, to establish a more precise link between each module and co-regulated cell types. The predominant cell types based on principal component 1 (PC1) were Hillock-like cells for ‘purple’, goblet cells for ‘black’, and B/T/mucous-ciliated cells for ‘light-yellow’ (FIG. 10A). Interestingly, the PC2 captured mucous-ciliated cells in both ‘purple’ and ‘black’ modules. Furthermore. both ‘purple’-PC2 and ‘black’-PC2 were connected to ‘light-yellow’-PC1, pointing to a potential role of mucous-ciliated cells in coordinating cellular interactions between these modules (FIG. 10B).

Elevated Mucous Ciliated Cell Frequency is Associated with Asthma Remission

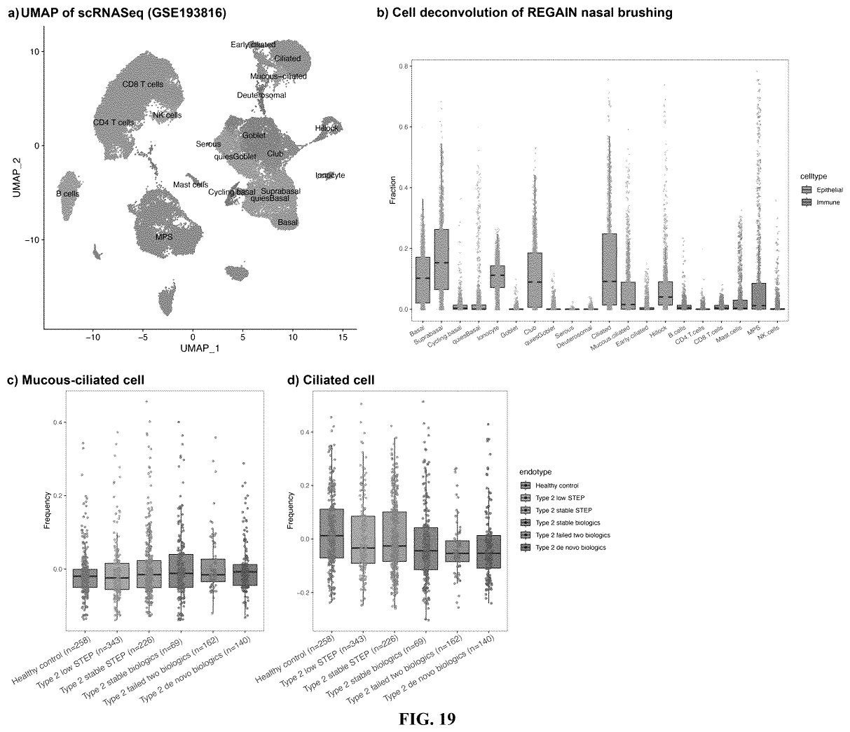

To strengthen the network-derived hypothesis that mucous-ciliated cells are required for response to therapies, bulk nasal brushing transcriptional data with deconvolution was used to explore cellular trajectories over time (FIG. 19A).

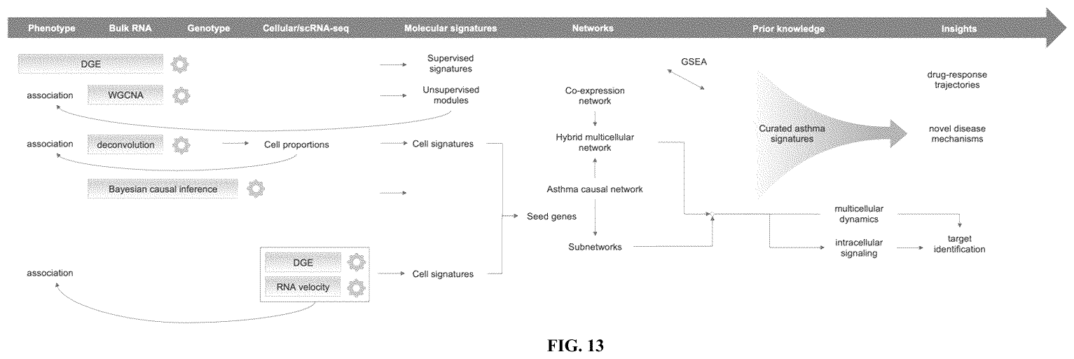

At baseline, the deconvolution analysis highlighted higher Hillock-like cell proportions and lower ciliated cell proportions in asthma patients compared to healthy controls, in line with the network results (FIG. 13).

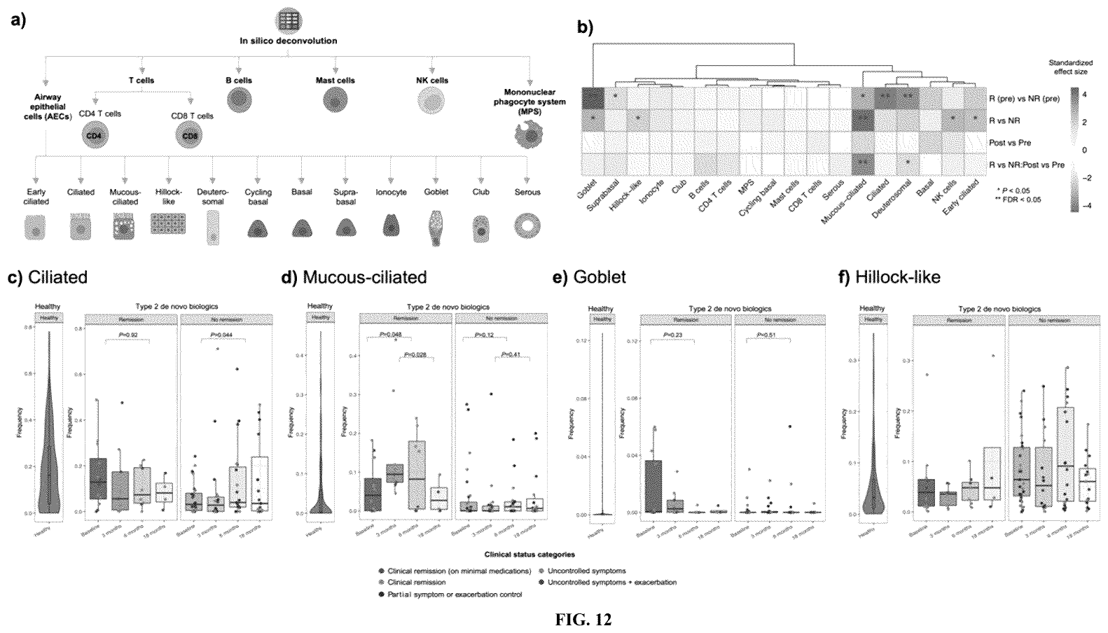

Next, the association of cell frequency over time with responses to biologics therapies in the ‘T2 de novo’ group was assessed. Intriguingly, while ciliated cell frequency at baseline was positively associated with remission (P=0.00046, FDR=0.011, FIG. 12B), it remained unchanged following treatment in remission participants (P=0.92, Wilcoxon signed-rank test, FIG. 12C), but rather increased in participants who did not achieve remission (P=0.044, from Wilcoxon signed-rank test, FIG. 12C). In contrast, mucous-ciliated cell frequency not only was nominally associated with remission at baseline (P=0.010, FDR=0.12, FIG. 12B), but also was the most strongly associated cell type distinguishing remission from non-remission in longitudinal analysis (P=0.00063, FDR=0.011, FIG. 12B), with a significant increase seen in remission participants (P=0.048, Wilcoxon signed-rank test, FIG. 3E) but not in non-remission (P=0.12, Wilcoxon signed-rank test, FIG. 3E). Interestingly, the increase of mucous-ciliated cells following biologics treatment was transient. There was a noticeable surge at 3-month and 6-month visits, followed by a decrease at the 18-month visit (P=0.028 from Wilcoxon signed-rank test comparing 18-month versus combined 3-month and 6-month visits, FIG. 3E). These results support the hypothesis that mucous-ciliated cells may be a dynamic cell state mediating cell transitions to enable improved airway function with treatment1. On the other hand, Hillock-like cell proportions was lower in remission across time (P<0.05, FIG. 12F), indicating that it may contribute to the poor outcomes in asthma patients following treatment.

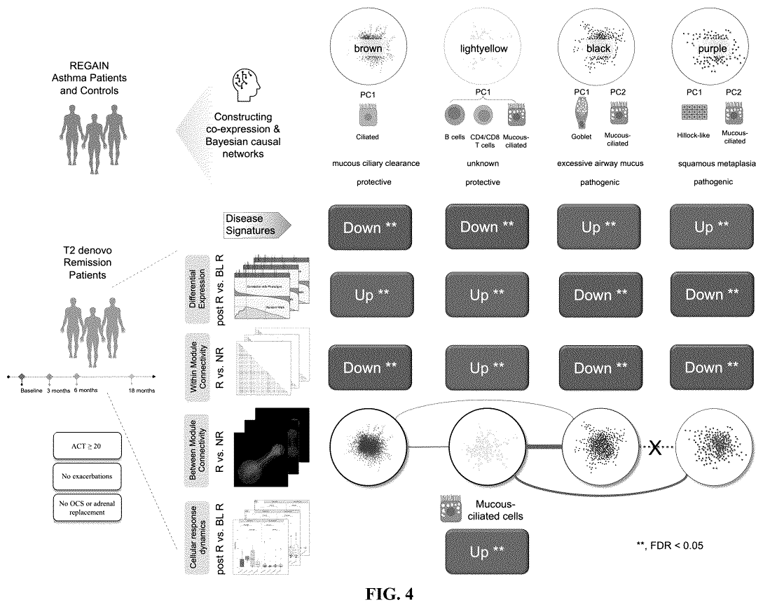

Together, these data demonstrate a novel primary regulatory mechanism for both asthma and responses to biologics, as summarized in FIG. 4. This mechanism is reflected in four co-regulated coexpression modules representing four cell types Analysis of REGAIN-derived asthma disease signatures revealed the pathogenic role of ‘purple’ (Hillock-like) and ‘black’ (goblet) modules, whereas ‘brown’ (ciliated) and ‘light-yellow’ (mucous-ciliated, B/T cells) modules appear to be protective. Further shown is the reversal of these changes in remission patients, as evident by the reduced expression and loss of connectivity of genes involved in the two pathogenic modules and vice versa. Among these cell types, a central role of mucous-ciliated cells in linking these four modules in treatment responses was identified. Furthermore, mucous-ciliated cells were the only cell type with specific increase in remission group following treatment. Therefore, a refined hypothesis that both goblet and Hillock-like cells can give rise to mucous-ciliated cells as part of the remission mechanism is proposed. The transition from goblet to mucous-ciliated cells has not been previously reported by others. Therefore, the relation between these two cell types was investigated.

Goblet Cells are Precursors of Mucous-Ciliated Cells after Recovery from IL-13 Treatment

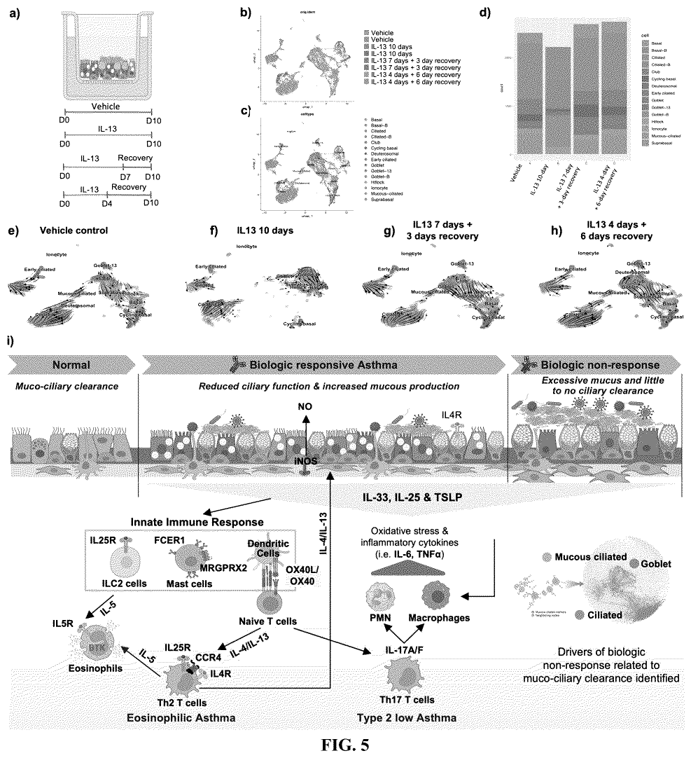

To validate the hypothesis derived from network and deconvolution analyses that goblet cells can give rise to mucous-ciliated cells during remission of asthma, an in vitro organoid model based on the use of air-liquid interface cultured airway epithelial cells (ALI) was set up. The model was exposed to different conditions using IL-13 cytokine to mimic type 2 inflammatory airway milieu, and its removal to mimic anti-IL-13 biologics treatment. The four conditions chosen were: A) vehicle; B) IL-13 for 10 days; C) IL-13 for 7 days followed by 3 days of recovery; D) IL-13 for 4 days followed by 6 days of recovery (FIG. 5A). Subsequently, cell suspensions were collected from each condition for scRNA-Seq analysis.

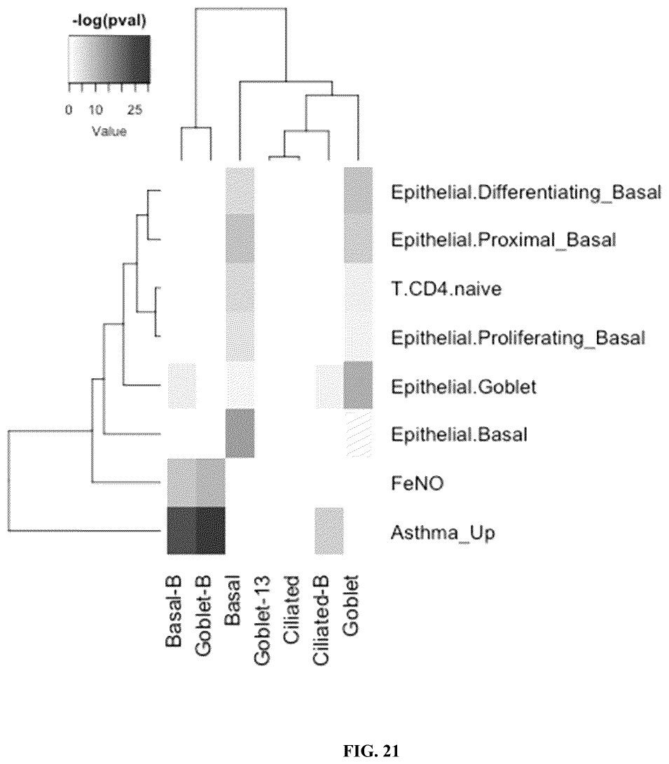

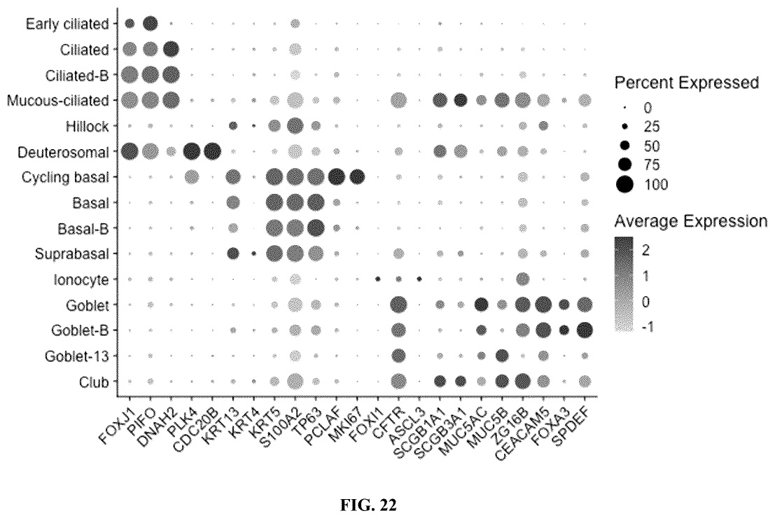

These data show that IL-13 promotes a considerable shift in ciliated and basal cell states, as evident by the appearance of unique IL-13 induced ciliated-B and basal-B cell clusters. Additionally, IL-13 induced a marked increase in the frequency of goblet cells (goblet-B; FIG. 5B, FIG. 21). Both goblet-B and basal-B cell clusters were enriched for a set of genes expressed in asthma bronchial epithelium and positively correlated with FeNO measures, consistent with their functional relevance to airway inflammation (FIG. 21). Interestingly, mucous-ciliated cells were markedly reduced after 10-day IL-13 stimulation (FIG. 5B, FIG. 5D and FIG. 22). However, after recovery from 7-day and 4-day IL-13 stimulation (to mimic therapeutic intervention), mucous-ciliated cells were again present, consistent with the clinical observations presented here (FIG. 5D).

To better understand the potential origin of mucous-ciliated cells after therapeutic intervention, RNA-velocity analysis was performed to infer temporal dynamics from scRNA-Seq data. Under steady state conditions (i.e., vehicle treated samples), mucous-ciliated cells were inferred to originate from club cells and to be precursors of ciliated epithelial cells (FIG. 5E). In the IL-13+ recovery groups (mimicking therapeutic intervention), mucous-ciliated cells were also inferred to be precursors of ciliated epithelial cells, but here they were originating from goblet cells (FIG. 5G, FIG. 5H). Together, these results indicate a novel mechanism for resolution of hyperplastic goblet cell mediated type 2 airway dysfunction, where these cells transdifferentiate into ciliated cells through a mucous-ciliated cell intermediate, (FIG. 5I).

### Discussion

The REGAIN study was designed to fill the gap between clinical trial and real-world understanding of asthma by using real-world longitudinal multi-omics to probe the heterogeneity of asthma, particularly remission in response to therapy with biologics. A data-driven and hypothesis-free approach was applied to analyzing airway gene expression and genotype data that simultaneously considers all clinical measurements as well as the correlation structure of the data and how it changes over time in the REGAIN participants with asthma starting treatment with biologics. To this end, a novel hybrid network analysis was developed that leveraged the deep information of coexpression networks and the statistically inferred causality inherent in Bayesian networks. Additionally, networks were built from longitudinal molecular data specific to remission and lack of remission in response to biologic treatment. Remission correlated with network modules that changed both their level of expression and connectivity within and between modules. Systematic winnowing of the network modules most significantly related to remission based on objective statistical thresholds allowed experiments to focus on a core set of four modules most related to remission in nasal brushing epithelium.

The four core airway epithelium modules represent four cell types involved in squamous metaplasia and mucociliary clearance in airway epithelium. Based on reversal of expression and connectivity changes between remission and non-remission, the ‘purple’ (Hillock-like) and ‘black’ (goblet) modules represent pathogenic states and the ‘brown’ (ciliated) and ‘light-yellow’ (mucous-ciliated, B/T cells) modules represent protective states. Crucially, all four modules are linked to mucous-ciliated cells, the only cell type with specific increase in remission group following treatment. Accordingly, the hypothesis that both goblet and Hillock-like cells can give rise to mucous-ciliated cells during remission contributing to resolution of squamous metaplasia and recovery of mucociliary clearance was investigated. Mucous-ciliated cells are a state of airway epithelial cells defined by co-expression of FOXJ1 and MUC5AC. Although little is known about these cells, their elevation in asthmatic patients and the findings that they can be derived from ciliated cells and then transition into goblet cells, a cell type associated with features of asthma including excessive airway mucus, led to the assumption that they may be related to severity of disease and possibly responsible for asthma pathology. However, the findings presented here indicate that the pathway from ciliated epithelium to mucus ciliated cells, then to goblet cells, is not unidirectional, and that mucous-ciliated cells may also arise during a transition from disease to healing.

The plastic nature of mucous-ciliated cells as an intermediate state between ciliated cells and goblet cells is supported by data from both REGAIN participants and an in vitro model using ALI cultured pseudostratified AECs treated with IL-13 to mimic asthma. In REGAIN participants with asthma, a transient increase in mucous-ciliated cell frequency was observed after starting treatment with biologic therapy that was greater in participants who experienced remission. This result was further supported at the molecular level through a hybrid Bayesian-coexpression network approach, which demonstrated that the intra- and inter-cellular signaling of mucous-ciliated cells significantly differed between remission versus non-remission. Furthermore, a signaling subnetwork enriched for both mucous-ciliated marker genes and IL-13-induced goblet marker genes was discovered, which may represent a transition state between these two cell types during remission. In the ALI model, IL-13 induced changes in basal, secretory and ciliated cell populations. Additionally, chronic IL-13 treatment for 10 days induced a loss of mucous-ciliated cells and a concomitant dominance of an IL-13-induced goblet cell cluster, suggesting that mucous-ciliated cell-state may be driven towards goblet cells. Two distinct therapeutic intervention scenarios were modelled by treating cells with IL-13 for 4 days followed by washing and discontinuing for 6 days or IL-13 treatment for 7 days followed by washing and discontinuing for 3 days. In both scenarios, scRNA-Seq data and RNA velocity analysis showed the re-appearance of mucous-ciliated cells after recovery from IL-13 treatment and predicted that these cells were derived from a unique goblet cell cluster that was only observed in the two recovery groups. These results indicated that mucous-ciliated cells may be derived from goblet cells during resolution of type 2 cytokine-induced airway inflammation and are concordant with our deconvolution and network analysis of nasal brushing samples from REGAIN participants exposed to biologic therapy.

In the REGAIN study, epithelial cells from nasal brushings were used as a surrogate for the bronchial epithelium due to the ease of obtaining such samples from patients longitudinally with safety and minimal discomfort at a large scale. This offered a low risk, reliable way to sample serially and assess response to therapy, in contrast to bronchoscopy, which carries increased risk and cost and is difficult to perform serially, especially in more severe patients. Using nasal brushings facilitated obtaining larger sample sizes for greater analytic power, particularly with more severe asthma groups. Previous studies where nasal brushing and bronchial brushing samples were performed in parallel support these samples as a reasonable surrogate for bronchial epithelium, as does the similarity between the bronchial ALI cultures and REGAIN nasal brushing results prevented here. The analysis of REGAIN data presented here indicate that nasal brushing RNA samples can capture substantial portions of airway biology and reveal novel insights into molecular mechanisms of treatment-induced remission and therefore are of great value for clinical use to monitor and stratify patients.

REGAIN included patients across the spectrum of asthma severity, treatments, and disease control. The size and composition of the cohort and the enrollment from point of care in a clinical setting supports generalizability of these findings. A real-world cohort such as REGAIN offers the ability to assess the response of patients to treatment in usual clinical care as opposed to the more restrictive patient population included in treatment clinical trials. When coupled with rich molecular data for healthy control participants and a continuum of control and treatment for patients with asthma, offers the potential to discover novel mechanisms of disease and remission in asthma. This study has identified mucous-ciliated cell activity as a novel cellular mechanism of repair associated with durable remission in response to biologic therapy that may highlight an important mechanism of action for treatment and a potential target for therapeutic development. The state of the airway epithelium plays a key role in the severity and natural history of asthma. Repeated cycles of epithelial injury and repair are linked to severity of disease and ultimately structural changes associated with airway remodeling and persistent airflow limitation, suggesting the importance of effective early treatment to achieve durable and meaningful responses in asthma. Interestingly, similar mechanisms are implicated in pathophysiology of chronic obstructive pulmonary disease (COPD), suggesting a conservation of these mechanisms in chronic airway diseases. More generally, the ability to associate molecular findings with longitudinal patient outcomes in a real-world setting and probe them in combination with in vitro cell biology indicates a potentially fruitful path to test the hypothesis that cellular dynamics and degree of reversibility of airway damage can enable more precisely tailored therapies for respiratory disease in the future.

### Methods

### REGAIN Study Design and Participant Recruitment

REGAIN is an 18-month closed cohort study of asthma intended to discover novel mechanisms of asthma pathogenesis and control that includes a baseline cross-sectional comparison to healthy controls. This section summarizes the study design features and study participant data most relevant to the analysis of nasal brushing RNA-Seq data obtained from 528 adult participants with mild-to-severe asthma and 263 healthy controls.

Study participants were recruited from patients receiving clinical care in pulmonary and allergy/immunology specialty clinics at two sites at the Mount Sinai National Jewish Health Respiratory Institute in New York City, New York and one site at National Jewish Health, Denver, Colorado.

Asthma diagnosis was determined based on specialist assessment (allergist or pulmonologist), supportive clinical data in the electronic medical record, and exclusion of other non-asthma pulmonary disease (Table 5). Study participants with asthma were eligible for the REGAIN study if they were 18 years old or older, used no current tobacco products or active inhalant and had less than a 10 pack-year smoking history. Participants with a clinical history or imaging results consistent with non-asthma lung disease were excluded.

TABLE 5
Functional description of cell types analyzed in deconvolution.

| Cell type | Relevance in Asthma |
|---|---|
| Ciliated cells | Ciliated cells accounting for 21% of nasal epithelial cells have been |
| | defined by FOXJ1 expression. |
| Early ciliated cells | Early ciliated cells, precursors of ciliated cells, are characterized by |
| | expression of DYNLT1 and FTH1 in contrast to ciliated cells. |
| Mucous-ciliated cells | Mucous-ciliated cells have been defined as a transient cell state of |
| | ciliated cells that is increased in asthma patients, co-express |
| | MUC5AC and FOXJ1, and contribute to mucous cell hyperplasia. |
| Deuterostomal cells | Epithelial precursors to multi-ciliated and ciliated cells. |
| Basal cells | Airway progenitor expressing KRT5 and p63 that are located near the |
| | basement membrane of the airways. |
| Cycling basal cells | Proliferating basal cells. |
| Supra-basal cells | Basal cells that are moving to the upper layers of the airways as they |
| | are differentiating into other airway cell types. |
| Quiescent Basal cells | Inactive basal cells in airways. |
| Goblet cells | Mucous secreting cells. |
| Quiescent Goblet cells | Inactive and homeostatic goblet cells in healthy lungs. |
| Club cells | Low columnar/cuboidal secretory cells with short microvilli. |
| Hillock Cells | Highly keratinized and proliferative cell that may promote airway |
| | repair. |
| Mononuclear | Family of cells composed of bone marrow progenitors, monocytes |
| Phagocyte Systems | and macrophages. |
| (MPS) | |
| Serous cells | Granular cells that secrete a wide array of anti-microbial proteins and |
| | water. |
| Ionocytes | Rare cell type that regulates salt balance and airway fluids. |

Healthy controls were eligible to participate in REGAIN if they were 18 years or older, had not smoked for more than 6 months (confirmed by saliva cotinine level <30 ng/ml), reported a history of less than 10 pack-years of tobacco use, had no current respiratory symptoms (cough, wheeze, dyspnea), and had no current lung disease or other significant medical comorbidity (Table 6 and Table 7).

TABLE 6
Inclusion and exclusion criteria for
REGAIN participants with asthma.

Inclusion criteria for REGAIN participants with asthma
Age ≥18
Clinical diagnosis of asthma verified by a specialist physician (allergist or
pulmonologist) and at least one of the following:
Historical variability of airflow limitation via standard reversibility criteria
(≥12%, 200 mL within past 10 years)
Historical methacholine challenge PC₂₀ ≤8 mg/ml (within past 10 years)
FEV₁ variability between two clinic visits of 20% or more within the past
5 years either with improvement according to appropriate therapy or
decrease in lung function upon withdrawal or decrease of therapy
Elevated FeNO ≥50 ppb at least once historically (within past 10 years)
≤10 pack per year personal tobacco use, and not a current smoker of
cigarettes or other inhalational tobacco (e-cigarettes, marijuana or other
inhalational drug use)
At least partial response of presenting symptoms to GINA Step 1-5 asthma
treatment (ICS or ICS/LABA)
Exclusion criteria for REGAIN participants with asthma
>10 pack per year tobacco, current smoker of cigarettes, e-cigarettes or
other inhalational drug use
Clinically significant lung disease based on imaging or clinical history
other than asthma as determined by investigators
Current pregnancy or breast feeding
Anemia with hemoglobin level <9 mg/dl at the time of study enrollment

TABLE 7
Inclusion and exclusion criteria for REGAIN healthy control participants.

Inclusion criteria for REGAIN healthy control participants
Age ≥18
No current smoking (quit >6 months previously) confirmed by saliva cotinine
level ≤30 ng/mL
≤10 pack year tobacco history
No current respiratory symptoms (cough wheeze, dyspnea)
No current lung disease or other significant medical comorbidity (see exclusion criteria
below)
Exclusion criteria for REGAIN healthy control participants
Age <18
Diagnosis of asthma, COPD, emphysema, bronchiectasis or other significant lung
disease
Treatment or a history of malignancy currently or within the past 5 years, except
completely treated in situ carcinoma of the cervix, completely treated and resolved non-
metastatic squamous or basal cell carcinoma of the skin?
Treatment of inflammatory disease or autoimmune disease such as lupus, rheumatoid
arthritis, inflammatory bowel disease, multiple sclerosis or others
Treatment with immune modulating medications for a health condition including any of
the following (systemic glucocorticoids, sirolimus, tacrolimus, anti-TNF abatacept,
azathioprine, methotrexate, cyclosporine, cyclophosphamide, immunoglobulin,
mycophenolate, rituximab, plaquenil or others)
Treatment with anti-IL5, anti-IgE and anti-IL4 receptor alpha biologics for non-asthma
indications
Vaccination within 4 weeks
Currently pregnant or breast feeding
History of clinically significant renal, hepatic, cardiovascular, metabolic, neurologic,
hematologic, ophthalmologic, gastrointestinal, uncontrolled diabetes, uncontrolled
infectious disease, HIV, cerebrovascular, or other significant medical illness or disorder
which in the judgment of the investigator could impact the participant as a healthy control
Saliva cotinine >30 ng/mL

Study participants were recruited at Mount Sinai, and had a single 12-month visit. A total of 20 study participants received a 12-month visit under this procedure. Enrollment was reopened and an additional site, National Jewish Health, Denver CO, was added.

### Clinical Parameter Assessment

Demographic variables in Table 1 and Table 2 were self-reported by study participants in response to a structured interview by clinical coordinators at the baseline study visit. Asthma medications, exacerbation history, and control were assessed in study participants with asthma by clinical coordinators via a structured interview at all visits and confirmed by chart review. Comorbidities were assessed via structured interview by clinical coordinators via a checklist of the following conditions: gastroesophageal reflux disease (GERD), rhinitis, sinusitis, sleep apnea, chronic cough/laryngeal hypersensitivity, paradoxical vocal fold motion/vocal cord dysfunction, hypertension, diabetes, dyslipidemia, coronary artery disease, congestive heart failure, osteoarthritis, rheumatoid arthritis, other connective tissue disease, low back pain, and cancer.

### Assessment Schedule and Asthma Group Assignment

At the baseline visit, participants with asthma were assigned to one of five asthma groups according to type 2 biology defined by clinical biomarkers, degree of asthma control, and current treatment, and underwent assessment and biospecimen sampling at baseline, 6 months, and 18 months (Table 1, FIG. 1, Table 8). Participants scheduled to initiate treatment with FDA approved biologics based on treating physician recommendation underwent additional sampling at 3 months. For asthma participants at each study visit, we conducted multi-modal longitudinal profiling, combining study collected clinical data, electronic medical records (EMR), and collection of biospecimens (nasal brushings and whole blood samples) for RNA-Seq in addition to banking for future analyses. At baseline and 6-month visits, asthma study participants had the option to contribute read-world data (RWD) modalities generated by inhaler sensors, peak flow monitor, and smart phone mobile application.

TABLE 8
Sample size of RNA-Seq profiles available
summarized by subgroup and study visit.

| Subgroup | Baseline | 3 months | 6 months | 18 months |
|---|---|---|---|---|
| Healthy control | 259 | 0 | 0 | 0 |
| T2 low STEP | 102 | 0 | 49 | 12 |
| T2 STEP | 171 | 0 | 120 | 55 |
| T2 bio | 132 | 0 | 75 | 23 |
| T2 failed | 33 | 2 | 22 | 13 |
| T2 de novo | 54 | 32 | 31 | 23 |
| Unclassified | 16 | 3 | 5 | 1 |

Healthy control participants contributed biospecimens and EMR data at the baseline visit in the same way as asthma participants but were not prospectively followed or offered use of the RWD modalities. All asthma and healthy control participants contributed a whole blood sample at baseline for DNA extraction and genotyping.

The five asthma groups are shown in Table 1. A study participant was classified as ‘type 2-high’ if they had FeNO ≥50 parts per billion (ppb), absolute eosinophil count (AEC) ≥300/μL at any clinical care time point, or eosinophilia by induced sputum or bronchoscopy (≥3%) if available historically. A study participant was classified as ‘type 2-low’ if they did not meet criteria for ‘type 2’, had FeNO persistently ≤25 ppb, and absolute eosinophil count (AEC) persistently ≤200/μL. ‘T2 low STEP’ participants were receiving GINA step 1-5 asthma treatment (may or may not be on maintenance inhaled steroid), but with no current maintenance oral corticosteroids (mOCS) or biologics. ‘T2 low STEP’ participants could have any level of control by ACT/GINA. ‘T2 STEP’ participants were on GINA step 1-5 therapy (may or may not be on maintenance inhaled steroid) but with no mOCS or biologics and had ACT ≥20 at time of enrollment. ‘T2 bio’ participants were on biologic therapy (see Table 1 for a list of therapies) for at least 4 months prior to enrollment. ‘T2 bio’ participants met one of the following threshold definitions of control: 1) ACT ≥20, 2) ACT 16-19 and with well or partly controlled status according to GINA, or 3) 50% reduction of mOCS dose for asthma after initiation of current biologic. ‘T2 de novo’ participants were not on an asthma biologic for 4 months prior to enrollment, had any level of ACT at time of enrollment, and could be on mOCS. ‘T2 failed’ participants had any level of ACT at time of enrollment. Treatment failure-broadly defined as incomplete clinical response, adverse effects, or intolerance to at least 2 classes of biologics (anti IL5/5R, anti IL4Rα, anti-IgE, anti-TSLP) was required to be assigned to this group. ‘T2 failed’ participants could be currently on STEP+/−mOCS or an asthma biologic. Any asthma participant who did not meet any of these criteria for either ‘type 2-low’ or ‘type 2’ was excluded from this analysis.

### Clinical Remission and Asthma Control Categories

Clinical remission and asthma control were grouped as shown in Table 3 based on a combination of ACT score, GINA symptom score, and exacerbation history assessed at REGAIN study visits with reference to study participant clinical charts.

### Samples Profiled by Visit and Group and Longitudinal Analysis of ‘T2 De Novo’ Participants

Samples and data from all asthma study participants were included in analysis of baseline data. For longitudinal analyses of ‘T2 de novo’ participants, 2 study participants who did not start biologic therapy before 6 months and one who did not continue biologic therapy through 6 months were excluded from the analyses. A total 44 ‘T2 de novo’ participants, who completed baseline and 6-month visits and contributed molecular data of at least one visit, were included in longitudinal analysis. The sample size of ‘T2 de novo’ participants contributing nasal brushing RNA-Seq data at each visit is listed in Table 8. The distribution of biologics taken at 6 months along with clinical outcomes in this group is reported in Table 9.

TABLE 9
Biologics taken at 6 months in ‘T2 de novo’ group.

| Control status at | | | | | | |
|---|---|---|---|---|---|---|
| 6 months | Omalizumab | Dupilumab | Benralizumab | Mepolizumab | Tezepelumab | Total |
| Clinical remission (on | 0 | 3 | 0 | 0 | 1 | 4 |
| minimal medications) | | | | | | |
| Clinical remission | 1 | 4 | 1 | 0 | 3 | 9 |
| Partial symptom or | 0 | 4 | 5 | 1 | 1 | 11 |
| exacerbation control | | | | | | |
| Uncontrolled | 2 | 5 | 4 | 1 | 0 | 12 |
| symptoms | | | | | | |
| Uncontrolled | 1 | 4 | 1 | 2 | 0 | 8 |
| symptoms + | | | | | | |
| exacerbation | | | | | | |
| Total | 4 | 20 | 11 | 4 | 5 | 44 |

### Biological Sample Collection and Molecular Profiling

Nasal brushings samples for nasal epithelial cells were collected by inserting a cytology brush 1-2 cm into the nares and rotating for 3-5 seconds behind the inferior turbinate. Brushes were placed in RNALater (ThermoFisher Scientific) for preserving RNA for processing and stored at −80° C.

### RNA Data Pre-Processing and OC

RNAlater tubes containing nasal brushings were thawed at 4° C. The brush was squeezed and scraped against the inner top edge of the tube, so all the liquid and cells were drained into the tube. Cells were then separated from the solution by centrifuge for 20 minutes at 3200×g, and total RNA was purified from the cells using RNeasy mini kit (QIAGEN). The amount of nasal brushing RNA was measured using nanodrop and fluorescent dye-based Qubit RNA quantitation. Ribo-Zero Plus kits (Illumina) were used for library preparation following the vendor recommended protocol. The Ribo-Zero library preparation protocol covers the whole range of transcriptome-wide coding and long non-coding RNA transcripts. RNA sequencing was run on Illumina NovaSeq 6000 platform at a 100 bp pair-end format. After completion of sequencing, the data were demultiplexed and subjected to Picard-based QC analysis. RNA-Seq data with a ≥Q30 value entered downstream analysis.

### Omics Data Preprocessing and OC

The sequencing reads from RNA-Seq were mapped to human reference genome (based on gene transfer format (GTF) file Homo_sapiens. GRCh37.75.gtf) using STAR (version: 2.7.5b)28. The raw read counts mapped to each gene were calculated by featureCounts (version v2.0.1)29. Samples of low quality were excluded, if 1) the samples had <20 million mapped reads; and/or 2) there was inconsistency between the self-reported birth-sex and the sex inferred based on the expression level of Y-linked genes (USP9Y and RPS4Y1). The sample-wise QC resulted in RNA-Seq profiles of 1,233 nasal brushing samples. Lowly expressed genes were removed from downstream analysis, if 1) the genes had <20 read counts in total across all samples; and/or 2) the genes had mapped reads in <2 samples. This resulted in 50,499 genes in nasal brushing data for downstream processing and analysis. Read counts were adjusted for gene length and GC content using the R package EDASeq (version 2.30.0′), where gene length and GC content were retrieved from Biomart, followed by trimmed mean of M-values (TMM) normalization adjusting for library size differences across samples using the calcNormFactors function in R package edgeR (version 3.38.1). The normalized read counts were then transformed to log 2-counts per million (log 2CPM) accounting for the mean-variance relationship in the data using the voom function of the R package limma (version 3.54.2). The batch effects were eliminated by the ComBat34 function in the sva R package (version: 3.44.0)35, followed by additional adjustment for age, sex, study site, batch, mapping rate, number of mapped reads as the covariates in a linear regression model implemented in the fitVarPartModel function of the R package variancePartition (version: 1.26.0).

### Genotype OC and Imputation

gDNA was isolated from whole blood using the QIAamp DNA Blood Kits (QIAGEN) and carried out genotyping using Illumina's Global Diversity Array (GDA-8v1-0). Scanned raw data was analyzed on Illumina's Genome Studio software, and high-quality data of sample-wise call rate ≥99% entered downstream analysis. From the genotype data, the sample's sex was inferred using PLINK software and confirmed with birth-sex recorded at the clinic. The MACH-minimac pipeline was applied for genotype imputation based on 1000 Genome Project reference. Quality of each imputed SNP was measured by the r2 statistic and SNP of r2<0.3 were removed.

### Differential Gene Expression Analysis

Differential gene expression (DGE) analysis was undertaken using the R limma-voom (version: 3.54.2)33 and nlme (version: 3.1-157) packages. The normalized (log 2CPM) and batch effect-corrected expression level of each gene was modeled. Cross-sectional disease biomarker analyses were undertaken for each gene by comparing baseline samples for asthma groups versus healthy controls. For each group, we fitted limma linear models to test for associations between genes and disease status. Predictive biomarker analysis was undertaken only on ‘T2 de novo’ participants, comparing baseline gene expression between dichotomized remission versus non-remission groups. To minimize the potential confounding effect of pre-treatment severity on remission, the model for baseline ACT scores was adjected. The other two biologics subgroups (‘T2 bio’ and ‘T2 failed’) were not used for predictive biomarker discovery, because they exhibited more homogeneous clinical status at 6 months. Similar to the baseline analysis, gene expression levels at 6 months were compared between remission versus non-remission. Efficacy biomarker analysis was also carried out using linear mixed-effects models with ‘T2 de novo’ samples, to investigate whether longitudinal changes in gene expression from baseline to 6 months were associated with remission versus non-remission. Participant ID was included as the random intercept. Due to low sample sizes at 6 months, baseline ACT scores were not adjusted in these models. The interaction term time×remission status was examined to identify significant remission-specific effects. Correction for multiple hypothesis testing was carried out using the Benjamini-Hochberg procedure. The criteria for significant DEGs and biomarkers was FDR <0.05 and absolute Log 2FoldChange >1. In comparisons with <50 asthma samples, including ‘T2 failed’ analysis and ‘T2 de novo’ remission analysis, a relaxed cutoff of nominal P<0.05 and absolute Log 2FoldChange >1 was applied.

### Collection of Asthma Related Signatures

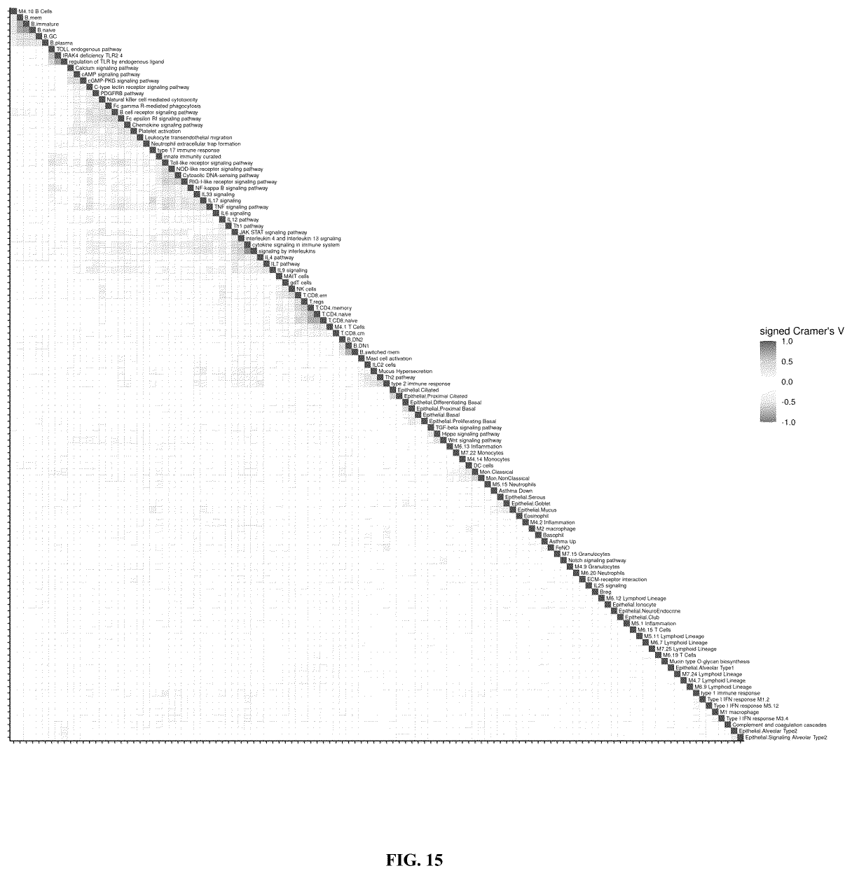

A list of 116 molecular signatures potentially related to asthma from databases and published literature was curated. These signatures reflected three cell type categories (epithelial, lymphoid, and myeloid), phenotypes, and signaling pathways. Next, to examine gene overlap between our curated signatures, a pairwise Cramer's V score (between 0 and 1) was computed using R rcompanion package (version: 2.4.34), signed by the log odds ratio from right-sided Fisher's exact test. GENCODE v.43 with 40,248 genes served as human genome background in the tests. Most of these gene sets had minimal overlap (FIG. 15). Functional associations were also assessed by computing a Gene Set Variation Analysis (GSVA) score for each signature across all nasal brushing RNA-Seq samples, followed by Pearson correlation analysis. The GSVA analysis was performed using R GSVA package (version: 1.44.5) with default parameters. Pearson correlation coefficient (r) was computed on the GSVA scores using the R cor.test function. Strong positive correlations were observed in some of the pairwise comparisons, and only a few signatures showed weak negative correlations with other signatures (FIG. 16). A selected set of 34 of the 116 signatures is shown in FIG. 2C and discussed above. These signatures served as references to understand how disease molecular pathologies are reflected in real-world asthma patient groups and during treatment. These signatures were also used to functionally characterize modules and subnetworks discovered in WGCNA and Bayesian causal networks.

### Functional Annotation of the ‘M4.2 Inflammation’ Signature

A subset of the curated signatures was from a blood transcriptomic study in human systemic lupus erythematosus (SLE), which were broadly annotated as inflammation-related gene sets. Among them, we were particularly interested in ‘M4.2 inflammation’, given its common association with both ‘type 2’ and ‘type 2-low’ asthma (FIG. 2C). To gain deeper mechanistic insights into this signature, an enrichment analysis was carried out with MSigDB C2 gene set collection using right-sided Fisher's exact test. It was discovered that this signature had the highest enrichment for leukocyte trafficking and innate immune responses gene sets (right-sided Fisher's exact test P=7.15×10-16 and P=9.36×10-14, respectively).

### Gene Set Enrichment Analysis

To identify molecular signatures associated with each asthma subgroup, and signature-level predictive and efficacy biomarkers, gene set enrichment analysis (GSEA) of the 116 curated asthma related signatures was performed using the R package clusterProfiler (version: 4.2.2)44, which implicitly invokes fgsea (version: 1.20.0). Coefficients (i.e., Log 2FoldChange) derived from linear differential expression models and interaction estimate from LME models were used to produce the input ranked list of genes. One million permutations were carried out to produce nominal p-values, and significant results were determined by an FDR <0.05. Normalized enrichment scores (NES) were used to assess directionality of the expression changes, indicating whether a given gene set was upregulated or downregulated in each comparison. Furthermore, to identify coexpression modules associated with each asthma subgroups and response to new biologics, we carried out GSEA of coexpression modules on differential expression (DE) signals using the same procedure above. Here, the differential expression (DE) signals of asthma patients and each group were characterized by identifying their gene expression changes at baseline from healthy controls (FIG. 2C and FIG. 6), and DE signals of response to new biologics by identifying gene expression changes between the ‘T2 de novo’ remission and non-remission groups at baseline, and between the baseline and post-biologic treatments within the remission group and non-remission group (FIG. 2C and FIG. 7).

Cell Type Marker Genes from scRNAseq Data

Cell type marker genes were obtained from the previous scRNAseq study. By default, the most significant 100 marker genes (based on area under the receiver operation curve (AUROC)) per cell type were selected. As for those with fewer than 100 significant markers, all available marker genes were collected. Because several cell types share many common marker genes with other cell types, we sought to identify mutually exclusive cell type markers for ciliated subtypes and goblet cells (i.e., mucous-ciliated, ciliated, and goblet cells) and basal subtypes (i.e., suprabasal and basal cells). To this aim, pairwise comparisons were performed among these cell types (mucous-ciliated vs. ciliated vs. goblet, and basal vs. suprabasal), as well as comparisons of each subtype against the rest, to identify differentially expressed genes for each subtype. The Findmarkers function within Seurat R package was used with an adjusted p-value threshold <0.05 and log 2FC>1.5. From this, 419, 173, and 285 marker genes were identified for ciliated, mucous-ciliated, and goblet cells, as well as 102 and 87 genes for basal and suprabasal cells, respectively.

### Coexpression Networks

Based on nasal brushing samples collected from asthma patients and healthy volunteers at baseline visit, a baseline coexpression network was constructed, the modules to establish baseline characteristics of the molecular networks in airway epithelium. To build coexpression networks for the participants in ‘T2 de novo’ group with and without clinical remission, respectively, we harnessed nasal brushing samples across study visits collectively to boost sample size while accounting for repeated measurements within individuals. A combined set of top 20,000 genes with highest average expression levels and the genes used in Bayesian network construction was chosen, with a total of 20,232 genes, followed by an inverse-normalization to Gaussian distribution for each gene. The R package WGCNA (version: 1.71) was used in the coexpression network construction, with soft power threshold automatically selected by the pickSoftThreshold function. For the reference network, the correlation matrix between pairs of genes was calculated by Pearson correlation, while for the remission status-specific networks, the correlation matrix was calculated by the function rmcorr_mat in the R package rmcorr (version: 0.6.0) to account for intra-individual correlation of repeated measures. The modules were built by hierarchical clustering on the topological overlap matrix (TOM) derived from the correlation matrix, requiring a minimum of 60 genes in each module.

### Annotation of Coexpression Modules

To understand the biological function of coexpression modules, an enrichment analysis was performed with 116 curated asthma-related gene sets and cell type marker genes using right-sided Fisher's exact test. 20 coexpression modules built on samples collected at the baseline visit on all asthma and healthy volunteer participants were annotated. To calculate FDR based on p-values, p.adjust function in R with Benjamini and Hochberg method was used. P-value 7.6×10-4 corresponded to FDR of 1% in this study.

### Principal Component Analysis to Annotate Coexpression Modules

To examine how each coexpression module was related to each cell type in detail, Principal Component Analysis (PCA) was performed for each module and each cell type marker. PCA is highly sensitive to outliers and can lead to misleading conclusions in the presence of outliers. Therefore, outliers were detected and removed before performing PCA on each module and each cell type markers. PCA was performed on all genes and identified outliers based on the Mahalanobis distances of PC1 and PC2, resulting in 10 outliers. The prcomp and Mahalanobis function from the stats packages in R were used. These outliers were excluded in the subsequent PCA on each module and each cell type markers. Given a gene set (i.e., each module or each cell type markers), PCA of gene expression profiles from asthma patients and healthy volunteers at baseline visit were calculated. Subsequently, the first two PCs (PC1 and PC2) from each module were compared to the first two PCs from each cell type marker. For this analysis, the correlation between PCs from each module and PCs from cell type markers were calculated. The significance (p value) and FDR of each correlation was also calculated. FDR was estimated from p-values by using p.adjust function in R with Benjamini and Hochberg method.

Differential Connectivity Analysis within Modules

To detect and quantify network reorganization between T2 de novo group with and without clinical remission, differential connectivity (DC) analysis between two networks was performed for each module. Given a set of N genes and two networks, x and y, the DC score was quantified among a set of genes (or module) as follows:

$$D {C}_{M} ( x , y ) = {\sum }_{i = 1}^{N - 1} {\sum }_{j = i + 1}^{N} {k}_{i j}^{x} - {\sum }_{i = 1}^{N - 1} {\sum }_{j = i + 1}^{N} {k}_{i j}^{y} ,$$

where, kᵢⱼ is the connectivity, i.e., topological overlap matrix (TOM) value, between two genes i and j in a given network. Given the nature of the coexpression network analysis, DC >0 indicates gain of connectivity (GOC) or enhanced coregulation between genes, whereas DC<0 indicates loss of connectivity (LOC) or reduced coregulation between genes. Specifically, we compared remission networks to non-remission networks. The significance level of the DC score can be accessed by permuting the data underlying two networks. Given N permutations, FDR is computed as follows:

$$F D R ( D {C}_{M} ) = \frac{1}{N} {\sum }_{p = 1}^{N} \vert D {C}_{M} ( x , y ) \vert > \vert D {C}_{M} ( {x}^{p} , {y}^{p} ) \vert ,$$

where x^{p} and y^{p} are the networks derived from the permuted data. 1000 times was permuted for each network by shuffling gene labels.

### Ranking Coexpression Modules

The modules were rank-ordered based on their enrichment across differential expression and network connectivity signatures to prioritize the most impacted modules for further investigation. Specifically, three differential expression (DE) components and one differential connectivity (DC) component were integrated. The first DE component consists of normalized module enrichment scores for differential expression signals derived from comparing all REGAIN asthma cases and controls (DE:Case vs. Ctrl in FIG. 3C). The second and third DE components consist of normalized module enrichment scores for differential expression signals comparing the ‘T2 de novo’ remission and non-remission groups at baseline (DE:R(pre) vs. NR(pre) in FIG. 3C), and comparing baseline and post-biologic (at 6 months) treatments within the R group (DE:post R vs. pre R in FIG. 3C). The DC component indicated DC scores comparing remission networks and non-remission networks (DC:R vs. NR in FIG. 3C). Because the scales of normalized enrichment scores and DC scores differed, each score was standardized using the standard deviation, and calculated the absolute values of the standardized scores. The mean of four absolute values of the standardized scores was used to rank the modules (Combined scores in FIG. 3C).

### Differential Connectivity Analysis Between Modules

Whether the proportion of edges between two modules were different between remission and non-remission hybrid networks was tested using a two-sample Z test of proportions. First, examined interaction (i.e., edges) was examined between two modules, M₁ and M₂ in each of the networks. Subnetworks of two modules, SN(M₁, M₂), were defined as the largest connected graph by projecting genes within two modules, g^{M}^{₁}^{,M}^{₂}ϵ(M₁∩M₂), onto each of the networks and all nodes that were no more than one layer away from these genes g^{M}^{₁}^{,M}^{₂} were identified. Additionally, the genes within the subnetworks of two modules, SN(M₁, M₂), were classified as subnetwork genes of M₁ and M₂, denoted as SN(M₁|M₁, M₂) and SN(M₂|M₁, M₂), respectively. To refine the classification, Super(M₁), Core(M₁), and Outer(M₁) genes of module M₁ were defined as follows:

Core(M₁)ϵ(M₁∩SN(M₁,M₂)),

Super(M₁)ϵ(SN(M₁)∩SN(M₁,M₂)),

Outer(M₁)ϵSuper(M₁)−Core(M₁),

where SN(M₁) represented genes in M₁, and their connected nodes that are no more than one layer away from genes in M₁. Similarly, Super, Core, and Outer sets of M₂ module were defined. Based on these sets of genes, a subnetwork of M₁ and M₂, SN(M₁|M₁, M₂) and SN(M₂|M₁, M₂), was defined as genes removing intersect of outer sets of M₁ and M₂ from super sets of M₁ and M₂ such that

$$\begin{matrix}SN ( {M}_{1} \vert {M}_{1} , {M}_{2} ) ∈ Super ( {M}_{1} ) - ( Outer ( {M}_{1} ) ∩ Outer ( {M}_{2} ) ) , \\ SN ( {M}_{2} \vert {M}_{1} , {M}_{2} ) ∈ Super ( {M}_{2} ) - ( Outer ( {M}_{1} ) ∩ Outer ( {M}_{2} ) ) .\end{matrix}$$

After identifying SN(M₁|M₁, M₂), and SN(M₂| M₁, M₂), the number of edges between nodes were counted in different sets: one node from SN(M₁|M₁, M₂), and the other node from SN(M₂ M1, M₂). Subsequently, the proportion of edges between modules was computed relative to the total edges among genes in SN(M₁, M₂). Next, this procedure was applied to two networks, and the proportion of between modules edges was compared using a two-sample Z proportion test.

### Construction of Bayesian Networks

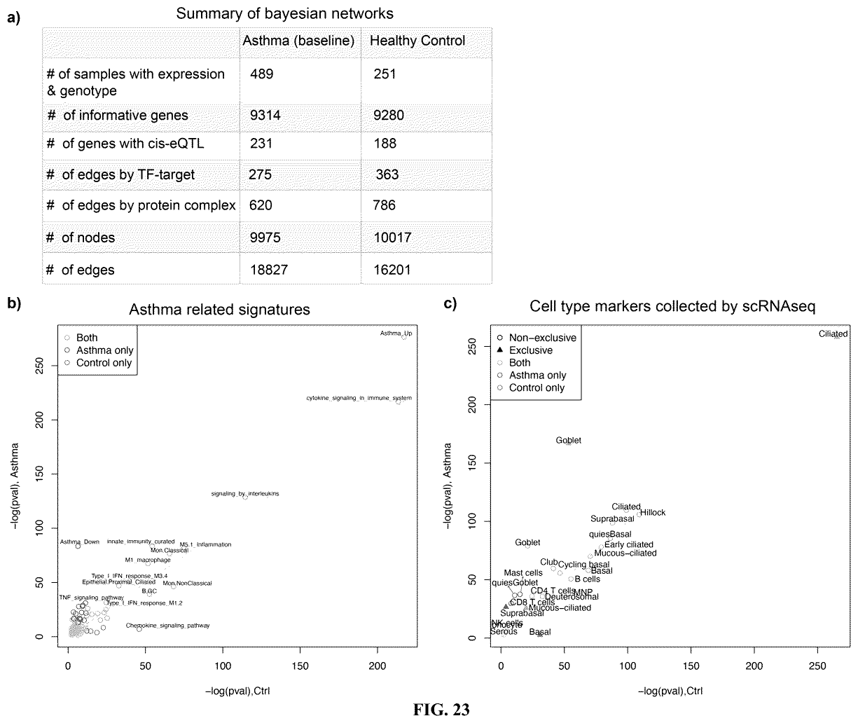

Nasal brushing-specific Bayesian causal networks were constructed based on genetic and baseline transcriptomic data. These networks were constructed separately for asthma and healthy control groups by using Reconstructing Integrative Molecular Bayesian Networks (RIMBANet) (FIG. 23A). Each causal network consists of ˜10,000 nodes and ˜15,000 putative causal relationships. Informative genes were selected for network construction based on their mean and variation. In total, ˜9,000 informative genes (mean >2 and variation >0.25, each corresponding to 75% quantiles of mean and variation) were included in the network reconstruction process for each network (FIG. 23A). cis-eQTL and transcription factor (TF)-target information were incorporated as priors such that cis-eQTLs or TF were parent nodes of the corresponding genes with cis-eQTLs or TF target genes. Also, protein complex information was considered as priors. It was determined that integrating genetic data such as cis-eQTL improved the quality of the network reconstruction by simulation51 and by experimental validations. Cytoscape 3.9 was used for network visualization. cis-eQTL, TF-target connections, and protein complex information were identified as follows.

Identification of cis-eQTL: cis-eQTLs were inferred separately in the asthma and healthy control using MatrixEQTL package. A linear regression model with covariates was used assuming an additive effect of genotypes to identify eQTLs, then checked whether eQTLs fall in proximity of genes. Population stratification was accounted for by adjusting for the inferred ancestry components, which corresponds to the first three principal components of the genotypes, as covariates. Gene expression and imputed genotypes, as well as the provided covariates, which included 3 ancestry components, sex, age, and race were used as follows: Expression˜Genotype+Age+Gender+Batch+Study Site+Race+PC1+ . . . +PC3, where PCk is the inferred kth ancestry component. cis-eQTLs was defined to be SNPs associated with genes where the distance between them is less than or equal to 1 Mb pairs of each other, and GRCh37 gene locations were used for consistency with the marker imputation panel. The variants with minor allele frequency (MAF) higher than 1% were used in calculation. P-value cutoff equal to 1×10⁻⁵ was used for detecting cis-eQTLs within 1 MB upstream of genes and 1×10⁻⁵ corresponded to FDR of 5% in this study.

Cis-eQTL were incorporated into the network model as priors. From 56,638 genes considered, we identified 359 and 290 genes with at least one significant cis-eQTL association at P-value of 1×10-5 for asthma participants and healthy control, respectively. Genes whose expression variation is not well explained by genotype (R2<0.05) were excluded. At the end, 231 and 188 cis-eQTLs were identified for asthma participants and healthy control, respectively, and were used as priors for Bayesian network (BN) constructions (FIG. 23A).

Identification of TF-target: Information on TF-target interactions was obtained from a previous study that assembled a collection of TF-target interactions for 1541 human TFs based on 1) manually curated repositories, 2) interactions derived from ChIP-seq binding data, 3) in silico prediction of TF binding on gene promoters, and 4) reverse-engineered regulons from large gene expression data sets. Among TF-target interactions, high confidence interactions were selected, defined as supported by all the four lines of evidence, manually curated by experts in specific reviews, or supported in at least two curated resources, resulting in 6080 TF-target interactions for 355 TFs. The TF-target interactions were refined using the following criteria. First, the activity of each transcription factor in the datasets was explored by examining its expression correlation with its target genes. Specifically, the target genes of each TF were compared to the genes showing significant expression correlation (correlation coefficients >0.4) with the gene encoding that TF. Active TFs were defined as those whose target genes exhibited enrichment of genes showing significant expression correlation. The significance levels of enrichment were measured by right-sided Fisher's exact test. Next, for active TFs, target genes whose expression exhibited a correlation greater than 0.4 with the transcription factor gene were selected. In total, 275 and 363 TF-target interactions for asthma and control networks were identified, respectively (FIG. 23A).

Identification of protein complex: Protein complex information was incorporated into the network model as priors. Protein complex information is obtained from Comprehensive Resource of Mammalian protein complexes (CORUM) database (website: mips.helmholtz-muenchen.de/corum). The protein-protein interactions were refined within protein complexes using the following procedure. First, human protein complexes with a membership count of 10 or more were selected. Next, pairwise correlations were computed among the genes that make up each protein complex and identified the gene with the highest mean correlation with other members as the driver gene. If a single gene was chosen as the driver in multiple protein complexes, it was retained as the driver for the complex with the highest mean correlation and assigned other genes with the next highest correlation as drivers for the remaining complexes. Then, membership genes whose correlation coefficients with the driver exceeded 0.4 were selected. Finally, protein complexes with more than five membership genes exhibiting strong correlations we considered (correlation coefficients >0.4) and the driver genes of the protein complexes along with their associated membership genes showing strong correlations were identified. Through this procedure, 620 and 786 driver-membership connections were identified within the protein complexes for asthma and control networks, respectively (FIG. 23A).

### Enrichment of Subnetworks for Asthma-Related and Cell Marker Gene Sets

The enrichment of 116 curated asthma-related gene sets and cell marker gene sets within the constructed global causal networks (BNashtma and BNctrl) were considered. For each gene set of interest (i.e., the original seed gene set), its corresponding subnetwork was identified as followed: given a set of original seed genes, its member genes were projected onto each global causal network and identified all of the nearest neighbors of these genes in the network. In the next step, direct connections (edges) among all the nodes in these pairs were identified and added. Then, the resulting significantly larger connected components than those expected by chance were used as subnetworks. The statistical significance (false discovery rate) was calculated by random permutation. A seed gene set of the equivalent size as the original seed gene set was randomly constructed, projected onto global causal network, and counted sizes of connected components. 1000 times random permutation was performed for each input gene set to estimate false discovery rate (FDR).

For each subnetwork, the enrichment of the original seed nodes in the subnetwork was evaluated by right-sided Fisher's exact test. The statistical significance (false discovery rate) for the enrichment was estimated by comparison to a null model generated by random permutations. Specifically, in each permutation, a seed gene set of the equivalent size as the original seed gene set was randomly constructed, and subnetwork analysis was performed to estimate its enrichment in the causal networks. This process was repeated 1000 times for each input gene set, to obtain an empirical null distribution of P values (which preserved the network structures and number of seed genes). This null distribution was then used to estimate false discovery rate (FDR). The results from individual gene sets with respect to each causal network are presented in FIG. 21.

### Construction of Hybrid Networks

To investigate whether the differential connectivity between modules were associated with remission, a hybrid network approach was developed. This approach combined a topological overlap matrix (TOM) generated by WGCNA, causal relationships generated by Bayesian networks, and betweenness centrality. In contrast to WGCNA networks, which only prioritize topological overlap and associative relationships, the integrative networks captured centrality and some inferred causal relationships as well that are important for assessing inter-module connectivity. The hybrid network was built as follows:

Based on TOM considering all pairwise and higher order correlation, the core overall correlation structure of the networks was formed.

Next, leveraging the Bayesian networks, edges that had causal support were prioritized.

Then, betweenness centrality to complement TOM based networks was considered by identifying those nodes and edges that play central bridging roles in the networks, which is referred to as edge pruning step. In the edge pruning step, cross-module connectivity leading to a more highly interconnected network but with less intra-module connectivity was prioritized. Also, in the edge pruning step, it was ensured that the topological features established for the WGCNA construction were preserved.

From WGCNA, a total of 21 modules were first detected from all baseline samples, including asthma and healthy control participants. Each module was then projected onto the hybrid network built on baseline samples from all asthma and healthy control participants (comprising 19,993 nodes), to more explicitly represent the connectivity structure within and between modules. ‘Induced’ hybrid subnetworks were subsequently constructed for each module by projecting module genes onto the network and identifying the largest connected subgraph comprised of nodes in the module directly connected to one another in the hybrid network. Each of the ‘induced’ hybrid subnetworks was annotated using cell type specific markers derived from published scRNA-Seq dataset.

To capture the temporal process of remission for ‘T2 de novo’ participants, and to understand the difference between remission and non-remission, the hybrid networks of these two groups built using all samples from multiple time points were compared, accounting for intra-individual correlation by employing a repeated measures methodology. This yielded a remission hybrid network comprised of 19,990 nodes and 48,071 edges, and a non-remission hybrid network comprised of 19,973 nodes and 47,770 edges. As above, induced hybrid subnetworks were derived for each of the modules of interest from the remission and non-remission networks and annotated them using cell type specific markers.

### In Silico Cellular Deconvolution Analysis

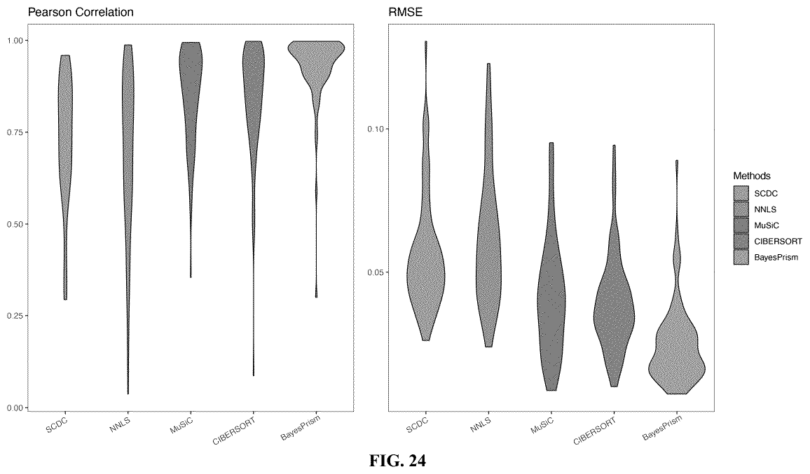

Cell deconvolution methods were applied to REGAIN nasal brushing bulk transcriptomic data. Many computational methods have been developed to infer cell proportions from bulk transcriptomics data. To select the computational method with best performance, five different deconvolution methods that use single-cell RNA-sequencing (scRNA-seq) data as a reference were evaluated: non-negative least squares (NNLS), MuSiC, SCDC, CIBERSORT, and BayesPrism. To assess the performance of methods, an independent validation scRNA-seq dataset was used for evaluation purposes that was different from the one applied for cell deconvolution in REGAIN nasal brushing data. Using a scRNA-seq dataset of human lung samples downloaded from GEO (accession number GSE136831), pseudo-bulk mixtures were generated in silico by aggregating counts of all single cells per individual to evaluate the deconvolution methods. The performance was assessed by means of Pearson correlation and root-mean-square error (RMSE) values between the cell type proportions computed by the different deconvolution methods and the known fraction of cell types in pseudo-bulk mixtures from scRNA-seq dataset. BayesPrism displayed the best performance based on Pearson correlation as well as RMSE values (FIG. 24). Therefore, BayesPrism was selected to infer cell compositions of REGAIN bulk transcriptomic data. To predict cell frequencies of nasal brushing data, recently published single cell RNA-seq (scRNA-seq) profiles from diverse immune and structural cells in asthmatic bronchial airway epithelium was used as a reference. The scRNA-seq dataset was downloaded from the Gene Expression Omnibus (GEO) under accession number GSE193816. The quality control and preprocessing procedures for the scRNA-seq data followed the guideline provided by the previous study and sub-clustering of airway epithelial cells was performed to recapitulate the 14 distinct structural and 6 distinct immune subsets identified in the previous study (FIG. 19A).

### Association Between Cell Frequencies and Asthma Subtypes

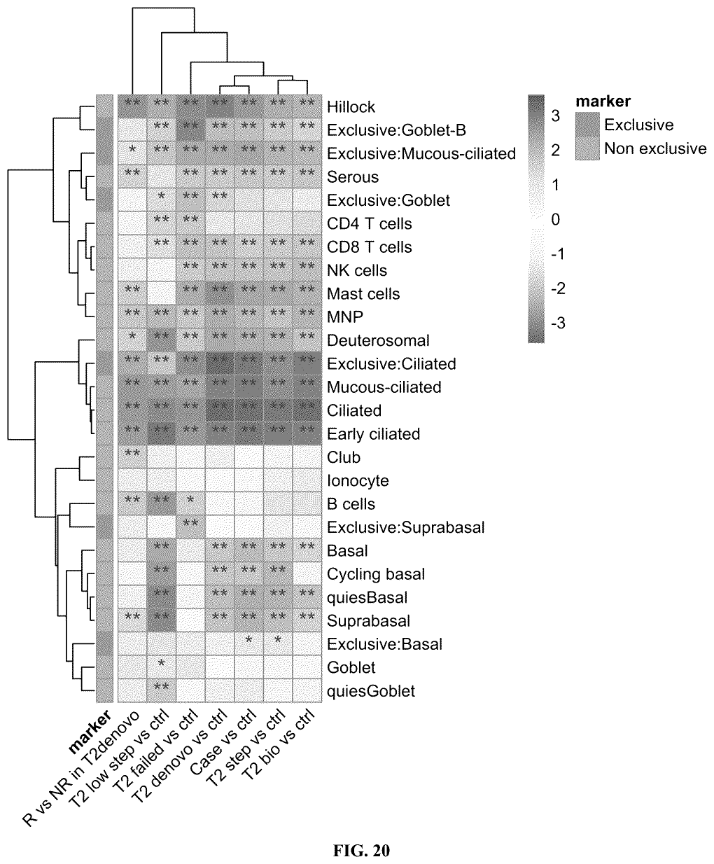

The association between cell frequencies and asthma groups pooled across visits was assessed using linear mixed-effect models, which included age, sex, study site, batch as covariates, and a random effect term to capture repeated measures of study participants. Each model was defined as follows: CellFreq ˜Age+Sex+Study Site+Batch+Asthma Group+(I|Participant), where ‘CellFreq’ indicates the cell frequency for each cell type, ‘Asthma Group’ represents a binary variable indicating each asthma group or a healthy control, and ‘Participant’ is a categorical variable representing study subjects. P-value and standardized coefficients from the asthma group term ‘Asthma Group’ were extracted. To calculate FDR based on p-value, p.adjust function in R with Benjamini and Hochberg method was used. GSEA of cell type markers was also performed to assess the association between cell frequencies and asthma subgroups. Cell type markers were derived from the scRNA-Seq data of asthmatic airway epithelium as described in the Methods (see “Cell type marker genes from scRNAseq data” for details) (FIG. 20).

Association of Cell Frequencies with Treatment and Remission Status in ‘T2 De Novo’ Study Participants

To identify cellular level efficacy biomarkers, the effects of treatment by remission status were investigated on cell frequencies in ‘T2 de novo’ study participants. These effects were tested by linear mixed-effects models with categorical variables for treatment, remission status, their interaction term, and a random effect term to account for repeated measurements of study participants. To facilitate the interpretation, we built two nested models and one full model with interaction effects as follows:

$$\begin{matrix}1 ) CellFreq \sim RvsNR + ( 1 \vert Participant ) ; \\ 2 ) CellFreq \sim PostvsPre + ( 1 \vert Participant ) ; or \\ 3 ) CellFreq \sim PostvsPre + RvsNR + PostvsPre \times RvsNR ( 1 \vert Participant ) .\end{matrix}$$

wherein ‘CellFreq’ was the cell frequency for each cell type, ‘PostvsPre’ indicated later visits or the baseline samples, ‘RvsNR’ signified whether participants achieved remission at 6 months or not, and ‘PostvsPre×RvsNR’ indicated the interaction effects. These variables were modeled as fixed effect terms, and Participant, the categorical variable coding independent subjects, is modeled as a random effect term. P-value and standardized coefficients of each fixed effect term were extracted and shown in FIG. 3D, consisting of ‘RvsNR’ from model 1, ‘PostvsPre’ from model 2, and the interaction term ‘PostvsPre:RvsNR’ from model 3. To control outliers, we applied a robust linear mixed-effects model using the rlmer function implemented in the robustlmm R package. Furthermore, to identify cellular level predictive biomarkers at baseline in ‘T2 de novo’, we applied multiple linear regression. This analysis assesses the relationship between baseline cell frequencies and remission status at 6 months with adjustment of baseline ACT score. To control outliers, a robust linear model was applied by using rlm function implemented in MASS R package. P-value and standardized coefficients of remission status term were extracted and are shown in FIG. 3D (R(pre)vsNR(pre)). Also, logistic regression was performed to predict remission status at 6 months based on baseline cell frequencies and ACT scores. To calculate FDR based on p-values, p.adjust function in R with Benjamini and Hochberg method was used.

### Generation of ALI Cultures

Human epithelial bronchial/tracheal lung cells (HBTEC) (Lifeline Cell Technology, Catalog #FC-0035) were thawed and incubated in a fibronectin-coated flask. When cells were at about 80% confluence, they were used to make human airway epithelial (HAE) air liquid interface cultures (ALIs). On the harvest day, 24 well transwell plates (Corning Life sciences, Catalog #3470) were coated with Collagen Type IV (Advanced BioMatrix, Catalog #5022-5MG) (100 μL on the apical well and 200 μL on the basal well) and incubated at 37° C. for two hours. Collagen was removed post-incubation, and 100 μL of Bronchial Epithelial Growth media (BEBM) (Stemcell, Catalog #CC-3170) was added to the apical wells and 650 μL to the basal wells. Human epithelial bronchial/tracheal lung cells were harvested from the flask using TrypLE express (ThermoFisher Catalog number #12605010). HBTEC cells were counted, and 50,000 cells were added to the apical side of the transwell to make the human airway epithelial (HAE) ALIs. The HAE ALIs were grown in 37° C. incubator and 5% CO₂. Two days post incubation, the culture medium was removed from apical and basal wells to convert to an Air-Liquid Interface (ALI) by adding 450 μL of PneumaCult-ALI media (Stemcell, Catalog #5001) to just the basal well, leaving the apical side of the well empty. HAE ALIs were maintained by changing media three times a week for two weeks and twice a week thereafter. Ciliation occurred around 21 days. Mucous production was monitored, and a mucus plug cleaning was performed every two weeks or as needed by adding 200 μL of DPBS without Ca++ and Mg++(Sigma-Aldrich, Catalog #D8662-500ML) for two hours and then aspirating the DPBS and changing the media on the basal side. The wells were used for further experimentation after two months.

The IL-13 treatment of HAE ALIs was done in four groups. Group A—Vehicle control (0.1% BSA in PBS), Group B—Chronic IL-13 treatment for 10 days, Group C—IL-13 treatment for 7 days followed by 3 days of no IL-13 treatment, and Group D—IL-13 treatment for 4 days followed by 6 days of no IL-13 treatment. Each treatment group was done in duplicate ALI transwells. IL-13 (R&D Systems, Catalog #213-ILB-010) was added at a concentration 100 μg/ml of media.

Processing Samples for scRNAseg of ALI Cultures

Transwell inserts were transferred to a clean 24 well plate. 2 mL of TRYPLE express (ThermoFisher Catalog number #12605010) was added to the basal side of well and 1 mL to the apical side. The organoids were incubated for 5-10 minutes at 37° C. and then gently pipetted up and down on both basal and apical membrane to detach the cells. The cell suspension was transferred to a 15 mL tube containing 5 mL of PneumaCult-ALI media (without hydrocortisone). The transwells were washed with 1 mL of HBSS and it was combined with the cell suspension solution. A microscope was used to confirm cells were detached from the transwell inserts. The cell suspension was centrifuged at 250 g for 5 minutes and then the cell pellets were gently resuspended in 500 μL of PneumaCult-ALI media (without hydrocortisone) with 1:40 diluted DNase 1 (Sigma Catalog #4536282001). For each condition, 2 transwell inserts were pooled together by combining the cell suspensions to the same tube. Samples were transferred to ice and cell viability was assayed. Samples were loaded on to Chromium connect sample deck and the 3′ gene expression protocol (Chromium connect protocol Version:CG000180 Rev D).

scRNAseq Data Preprocessing and Clustering of ALI Cultures

Raw FASTQ files generated by scRNA-Seq from each sample were respectively preprocessed with 10× Genomics Cell Ranger (version 7.2.0) based on human genome assembly GRCh38 and GENCODE release 3264, and the raw read count per transcript per cell were generated by the “count” subcommand of Cell Ranger. The filtered gene-barcode matrix from Cell Ranger output was used, which excluded barcodes that correspond to the background noise (e.g. free-floating mRNA from lysed or dead cells).

Next a more stringent cutoff was applied to retain high quality cells: cells with >35% mitochondrial genes or <200 unique genes detected or >0.25 doublet prediction score from scrublet 0.2.365 were deemed low-quality cells and were filtered out of matrix prior to proceeding with downstream analyses. The percentage of mitochondrial genes was computed using 13 mitochondrial genes. Downstream analysis was performed using Seurat version 5.0.166. The counts for each remaining cell in the matrix were ‘LogNormalized’ using NormalizeData( ) function using Seurat. For clustering, 2000 highly variable genes were selected using ‘vst’ method in the FindVariableFeatures function and used as input for principal component analysis (PCA). To account for technical variability between experiments, the resulting principal component scores were aligned using the Harmony algorithms. The top 50 principal components were used as input for Louvain clustering and Uniform Manifold Approximation and Projection (UMAP) for visualization. Cell type identification of each cluster was performed by using cell markers. Cell type annotation of each cluster was further confirmed as follows. For each cluster, we used the FindAllMarkers function to identify positive markers for that cluster. The positive markers for each cluster were compared to the most significant 100 cell type marker genes obtained from the previous scRNAseq study 15 by right-sided Fisher's exact test.

The marker genes of ciliated, basal and goblet subclusters shown in FIG. 24 were further identified as follows. Goblet subcluster marker genes were identified as differentially expressed genes across three comparisons, goblet versus goblet-B, goblet versus goblet-13, and goblet-B versus goblet. FindMarker function was used to perform each comparison, selecting significant positive markers with a Bonferroni adjusted P<1×10-10. Similarly, marker genes for basal (i.e., basal, basal-B) and ciliated subclusters (i.e. ciliated, ciliated-B) were defined in analogous manners.

RNA Velocity Analysis of scRNA-Seq

Aligned scRNA-seq reads were further processed using Velocyto (version 0.17) read counting pipeline. The GRCh38 expressed repeat mask was used. The output loom file was processed using Scanpy. The scVelo package (0.3.1) was applied to calculate the RNA velocity of scRNA-seq data with n_neighbors=20 parameter.

### Example II

### A System Suitable for Use in Practicing Various Embodiments of the Disclosure

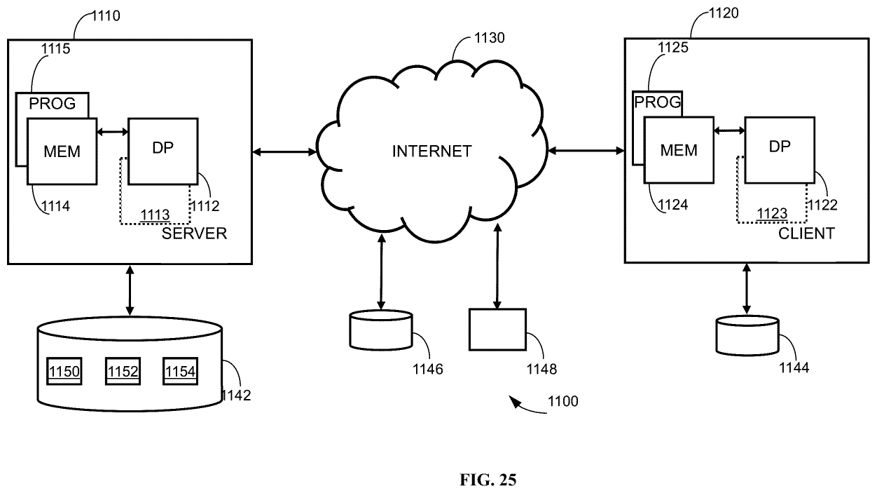

FIG. 25 shows a block diagram of a system 1100 that is suitable for use in practicing various embodiments. In the system 1100 of FIG. 25, the server 1110 includes a controller, such as a data processor (DP) 1112 and a computer-readable medium embodied as a memory (MEM) 1114 that stores computer instructions, such as a program (PROG) 1115. Server 1110 may communicate with a client 1120, for example, via the internet 1130.

Client 1120 includes a controller, such as a data processor (DP) 1122 and a computer-readable medium embodied as a memory (MEM) 1124 that stores computer instructions, such as a program (PROG) 1125. Server 1110 and/or client 1120 may also include a dedicated processor, for example a matrix processor 1113, 1123. Both server 1110 and/or client 1120 may communicate with a remote data processor 1148, for example, via the internet 1130 (as shown), and/or via direct communications channels (such as a wireless connection or a physical connection).

Databases 1142, 1144, 1146 may be connected directly to the server 1110, the client 1144 or the internet 1130. As shown, database 1142 stores patient data 1150, topology overlap matrices 1152 and Bayesian networks 1154; however, this information may be stored separately (or together) in any of the databases 1142, 1144, 1146.

The programs 1115, 1125 may include program instructions that, when executed by the DP 1112, 1122, enable the server 1110 and/or client 1120 to operate in accordance with an embodiment. That is, various embodiments may be carried out at least in part by computer software executable by the DP 1112 of the server 1110, the DP 1122 of the client 1120, by hardware, or by a combination of software and hardware.

In general, various embodiments of the server 1110 and/or client 1120 may include tablets and computers, as well as other devices that incorporate combinations of such functions.

The MEM 1114, 1124 and databases 1142, 1144, 1146 may be of any type suitable to the local technical environment and may be implemented using any suitable data storage technology, such as magnetic memory devices, semiconductor based memory devices, flash memory, optical memory devices, fixed memory and removable memory. The DP 1112, 1122 may be of any type suitable to the local technical environment, and may include general purpose computers, special purpose computers, microprocessors and multicore processors, as non-limiting examples.

1. A method of treating a chronic airway disease in a subject, comprising selecting a subject having increased goblet cell expression relative to a control and administering to the subject a drug that promotes transdifferentiation of goblet cells into ciliated cells.

2. The method of claim 1, wherein the chronic airway disease is selected from the group consisting of asthma, chronic rhinosinusitis, allergic rhinitis, allergic fungal rhinosinusitis, chronic sinusitis, allergic bronchopulmonary aspergillosis (ABPA), bronchiectasis, unified airway disease, eosinophilic granulomatosis with polyangiitis (EGPA), gastroesophageal reflux disease (GERD), cystic fibrosis (CF), chronic obstructive pulmonary disease (COPD), eosinophilic esophagitis (EoE), chronic rhinosinusitis with nasal polyps (CRSwNP), chronic rhinosinusitis without nasal polyps (CRSsNP), aspirin hypersensitivity, NSAID exacerbated respiratory disease (NSAID-ERD), perennial allergic rhinitis (PAR), food allergy, and chronic eosinophilic pneumonia (CEP).

3. The method of claim 1, wherein:
the chronic airway disease is asthma;
the drug is a biologic compound that is selected from the group consisting of dupilumab, benralizumab, mepolizumab, omalizumab, reslizumab and tezepelumab;
the goblet cells are hyperplastic goblet cells;
the ciliated cells are mucous ciliated cells;
airway damage is reversed in the subject after administering the drug;
the subject achieves a decreased Global Initiative for Asthma (GINA) score, an increased asthma control test (ACT) score, or a decrease in number of exacerbations within six months of administering the drug; or
the subject achieves asthma remission, or any combination thereof.

4-9. (canceled)

10. A method of determining whether a subject is a suitable candidate for drug treatment for a chronic airway disease, or a method of monitoring efficacy of drug treatment in a subject having a chronic airway disease, comprising:
obtaining a pre-treatment sample from the subject;
treating the subject with the drug; and
obtaining a post-treatment sample from the subject, wherein the subject is determined to be a suitable candidate for treatment if mucous-ciliated cell frequency is increased in the post-treatment sample relative to the pre-treatment sample.

11. The method of claim 10, wherein:
the pre-treatment and post-treatment samples are nasal brush samples from the subject,
the chronic airway disease is selected from the group consisting of asthma, chronic rhinosinusitis, allergic rhinitis, allergic fungal rhinosinusitis, chronic sinusitis, ABPA, bronchiectasis, unified airway disease, EGPA, GERD, CF, COPD, EoE, CRSwNP, CRSsNP, aspirin hypersensitivity, NSAID-ERD, PAR, food allergy, and CEP;
the chronic airway disease is asthma;
the drug is a biologic compound that is selected from the group consisting of dupilumab, benralizumab, mepolizumab, omalizumab, reslizumab and tezepelumab;
the goblet cells are hyperplastic goblet cells;
the ciliated cells are mucous ciliated cells; or
the subject achieves asthma remission after administering the drug, or any combination thereof.

12-24. (canceled)

25. A method of screening whether a drug is a suitable treatment for a chronic airway disease, comprising contacting goblet cells with the drug, wherein the drug stimulates transdifferentiation of goblet cells into ciliated cells if the drug is suitable for treating the chronic airway disease, optionally wherein:
the goblet cells are obtained from nasal brush samples from the subject;
the goblet cells are grown in vitro;
the chronic airway disease is selected from the group consisting of asthma, chronic rhinosinusitis, allergic rhinitis, allergic fungal rhinosinusitis, chronic sinusitis, ABPA, bronchiectasis, unified airway disease, EGPA, GERD, CF, COPD, EoE, CRSwNP, CRSsNP, aspirin hypersensitivity, NSAID-ERD, PAR, food allergy, and CEP;
the chronic airway disease is asthma;
the drug is a biologic compound that is selected from the group consisting of dupilumab, benralizumab, mepolizumab, omalizumab, reslizumab and tezepelumab;
the goblet cells are hyperplastic goblet cells; or
the ciliated cells are mucous ciliated cells, or any combination thereof; or
(II) a bioassay to screen whether a drug is a suitable treatment for a chronic airway disease in a subject, comprising contacting goblet cells from the subject with the drug, wherein the drug stimulates transdifferentiation of goblet cells into ciliated cells if the drug is suitable for treating the chronic airway disease, optionally wherein:
the drug is administered directly to the subject;
the goblet cells are obtained from nasal brush samples from the subject;
the goblet cells are obtained from nasal brush samples from the subject prior to administering the drug and grown in vitro;
the goblet cells grown in vitro are contacted with the drug;
the chronic airway disease is selected from the group consisting of asthma, chronic rhinosinusitis, allergic rhinitis, allergic fungal rhinosinusitis, chronic sinusitis, ABPA, bronchiectasis, unified airway disease, EGPA, GERD, CF, COPD, EoE, CRSwNP, CRSsNP, aspirin hypersensitivity, NSAID-ERD, PAR, food allergy, and CEP;
the chronic airway disease is asthma;
the drug is a biologic compound that is selected from the group consisting of dupilumab, benralizumab, mepolizumab, omalizumab, reslizumab and tezepelumab;
the goblet cells are hyperplastic goblet cells; or
the ciliated cells are mucous ciliated cells, or any combination thereof.

26-42. (canceled)

43. A method for predicting whether a subject with asthma will respond to asthma therapy, or will achieve asthma remission in response to asthma therapy, comprising:
measuring an expression level of at least one gene selected from the group consisting of C20orf85, FAM92B, CAPS, MORN5, CAPSL, ZMYND10, SNTN, TCTEX1D2, PIFO, RSPH1, DYNLRB2, C2orf40, SPA17, CCDC170, C11orf88, CETN2, ROPN1L, CCDC78 and C9orf24 in a biological sample from the subject,
wherein an increase in expression level of at least one gene selected from the group consisting of C20orf85, FAM92B, CAPS, MORN5, CAPSL, ZMYND10, SNTN, TCTEX1D2, PIFO, RSPH1, DYNLRB2, C2orf40, SPA17, CCDC170, C11orf88, CETN2, ROPN1L, CCDC78 and C9orf24 relative to a control is an indication that the subject will respond to the asthma therapy, or is an indication that the subject will achieve asthma remission from the asthma therapy; or
(II) a method for predicting whether a subject with asthma will achieve asthma remission in response to asthma therapy comprising:
measuring a ciliated cell gene signature in a biological sample from the subject,
wherein an increase in the ciliated cell gene signature expression levels observed relative to a control is an indication that the subject will achieve asthma remission from the asthma therapy; or
(III) a method of treating asthma in a subject with an increased expression level of a ciliated cell gene signature in a biological sample obtained from the subject relative to a control, comprising selecting the subject with an increased ciliated cell gene signature and administering to the subject asthma therapy.

44. The method of claim 43, wherein:
the asthma therapy comprises administering a biologic compound to the subject, optionally wherein the biologic compound is selected from the group consisting of dupilumab, benralizumab, mepolizumab, omalizumab, reslizumab and tezepelumab;
the asthma therapy increases Asthma Control Test (ACT) score, reduces Global Initiative for Asthma (GINA) score, and/or decreases risk of exacerbations in the subject; or
the biological sample is a nasal brush sample.

45-52. (canceled)

53. The method of claim 1, wherein the subject has an increased expression level of at least one gene selected from the group consisting of C11orf88, C20orf85, C2orf40, C9orf24, CAPS, CAPSL, CCDC170, CCDC78, CETN2, DYNLRB2, FAM92B, MORN5, PIFO, ROPN1L, RSPH1, SNTN, SPA17, TCTEX1D2 and ZMYND10 in a biological sample obtained from the subject relative to a control, further comprising selecting the subject with the increased expression level and administering to the subject asthma therapy.

54. The method of claim 53, wherein:
the asthma therapy comprises administering a biologic compound to the subject;
the biologic compound is selected from the group consisting of dupilumab, benralizumab, mepolizumab, omalizumab, reslizumab and tezepelumab;
the asthma therapy increases ACT score, reduces GINA score, and/or decreases risk of exacerbations in the subject; or
the biological sample is a nasal brush sample, or any combination thereof.

55-58. (canceled)

59. The method of claim 43, wherein the ciliated cell gene signature is an epithelial ciliated signature, optionally wherein the epithelial ciliated signature comprises genes selected from the group consisting of FAM92B, CAPSL, C20orf85, C11orf88, SNTN, ROPN1L, ZMYND10, C9orf24, MORN5, CCDC78, CCDC170, RSPH9, C9orf116 and RSPH1.

60. (canceled)

61. The method of claim 43, wherein the ciliated cell gene signature is an epithelial proximal ciliated signature, optionally wherein the epithelial proximal ciliated signature comprises genes selected from the group consisting of CAPSL, C20orf85, SNTN, CAPS, ROPN1L, PIFO, ZMYND10, C9orf24, MORN5, C2orf40, WDR54, CRIP1, DYNLRB2, SPA17, CES1, C9orf116, RSPH1, TCTEX1D2, TSPAN1, CETN2, TUBB4B and DYNLT1.

62. (canceled)

63. The method of claim 43, wherein ciliated cell frequency is increased in the subject relative to the control.

64. The method of claim 43 wherein the asthma therapy comprises administering a biologic compound to the subject, optionally wherein the biologic compound is selected from the group consisting of dupilumab, benralizumab, mepolizumab, omalizumab, reslizumab and tezepelumab.

65. (canceled)

66. The method of claim 60, wherein:
the asthma therapy increases ACT score, reduces GINA score, and/or decreases risk of exacerbations in the subject;
the biological sample is a nasal brush sample; or
the ciliated cell gene signature comprises one or more genes selected from the group consisting of FAM92B, CAPSL, C20orf85, C11orf88, CAPS and RSPH9.

67-69. (canceled)

70. The method of claim 43, wherein the ciliated cell gene signature is an epithelial ciliated signature, optionally wherein the epithelial ciliated signature comprises genes selected from the group consisting of FAM92B, CAPSL, C20orf85, C11orf88, SNTN, ROPN1L, ZMYND10, C9orf24, MORN5, CCDC78, CCDC170, RSPH9, C9orf116 and RSPH1.

71. (canceled)

72. The method of claim 43, wherein the ciliated cell gene signature is an epithelial proximal ciliated signature, optionally wherein the epithelial proximal ciliated signature comprises genes selected from the group consisting of CAPSL, C20orf85, SNTN, CAPS, ROPN1L, PIFO, ZMYND10, C9orf24, MORN5, C2orf40, WDR54, CRIP1, DYNLRB2, SPA17, CES1, C9orf116, RSPH1, TCTEX1D2, TSPAN1, CETN2, TUBB4B and DYNLT1.

73. (canceled)

74. The method of claim 43, wherein ciliated cell frequency is increased in the subject relative to the control.

75. The method of claim 43 wherein the asthma therapy comprises administering a biologic compound to the subject, optionally wherein the biologic compound is selected from the group consisting of dupilumab, benralizumab, mepolizumab, omalizumab, reslizumab and tezepelumab.

76. (canceled)

77. The method of claim 69, wherein:
the asthma therapy increases ACT score, reduces GINA score, and/or decreases risk of exacerbations in the subject,
the biological sample is a nasal brush sample; or
the ciliated cell gene signature comprises one or more genes selected from the group consisting of FAM92B, CAPSL, C20orf85, C11orf88, CAPS and RSPH9.

78. (canceled)

79. (canceled)
